(19) United States
(12) Patent Application Publication
(10) Pub. No.: US 2016/0122307 A1
(43) Pub. Date: May 5, 2016

(54) PERHYDROQUINOXALINE DERIVATIVES

(71) Applicant: DR. AUGUST WOLFF GMBH & CO. KG ARZNEIMITTEL, Bielefeld (DE)

(72) Inventors: CHRISTOPH ABELS, BIELEFELD (DE); ULRICH KNIE, BIELEFELD (DE); MICHAEL SOEBERDT, BIELEFELD (DE)

(73) Assignee: DR. AUGUST WOLFF GMBH & CO. KG ARZNEIMITTEL, BIELEFELD (DE)

(21) Appl. No.: 14/890,183

(22) PCT Filed: May 16, 2014
(86) PCT No.: PCT/EP2014/060113

§ 371 Date: Nov. 10, 2015

(30) Foreign Application Priority Data
May 17, 2013 (EP) 13168208.0

(51) Int. Cl. C07D 241/42; C07D 405/14; C07D 409/14; A61K 31/506; C07D 401/14; A61K 45/06; A61K 31/498; C07D 403/02; C07D 403/14

(52) U.S. Cl. CPC C07D 241/42; C07D 403/02; C07D 405/14; C07D 409/14; C07D 403/14; C07D 401/14; A61K 45/06; A61K 31/498; A61K 31/506

(57) ABSTRACT

The present invention relates to perhydroquinoxaline compounds according to the general formula (1), their use as a medicament, in particular as analgesic, antipruritic and anti-inflammatory agents, and their preparation.

The present invention relates to perhydroquinoxaline derivatives and medicaments containing perhydroquinoxaline derivatives, particularly for use as analgesics, antipruritic and antiinflammatory agents.

Treatment of pain is of great importance in medicine. Analgesic agents as a rule act by activating opioid receptors. Conventional opioids, such as morphine, are thus opioid analgesics which are often employed in clinical pain therapy because of their potent analgesic action. These activate the μ receptor. However, undesirable side effects of such pain therapy are sometimes considerable centrally mediated side effects, such as respiratory depression, vomiting and bradycardia. Possible psycho-dependencies are furthermore a disadvantage.

In view of the large number of types of pain and inflammation and diseases associated with pain and inflammation, there is a great need for new active agents to treat these symptoms.

WO2009/080745 relates to perhydroquinoxaline derivatives useful as analgesic agents.

The invention was based on the object to provide novel compounds which can be used as pharmaceutical active compounds, in particular for combating pain, pruritus and inflammation.

This object is achieved by the provision of perhydroquinoxaline compounds according to the general formula (I) as shown below or a solvate or hydrate thereof or a pharmaceutically acceptable salt thereof:

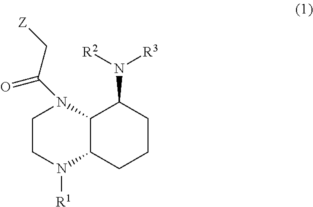

wherein:

R¹ is chosen from the group comprising H; C₁-C₁₀-alkyl; C₃-C₁₀-cycloalkyl; (COO(C₁-C₁₀-alkyl);
phenylalkyl with C₁-C₆-alkyl, wherein the phenyl radical can be substituted by one or more identical or different groups chosen from the group comprising halogen, C₁-C₆-alkyloxy, NH₂, NH(C₁-C₅-alkyl), N(C₁-C₅-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl);
C₁-C₁₀-acyl; heterocyclylacyl containing one, two, three or four hetero atoms chosen from the group comprising NH, O and/or S; phenylacyl, wherein the acyl radical is a C₁-C₆-acyl radical and the phenyl radical can be substituted by one or more identical or different groups chosen from the group comprising halogen, C₁-C₆-alkyloxy, COO(C₁-C₆-alkyl), NH₂, NH(C₁-C₅-alkyl), N(C₁-C₅-alkyl)₂, CONH₂, CONH(C₁-C₆-alkyl), CON(C₁-C₆-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl);
mono-, bi- or tricyclic heteroaryl containing one, two, three or four hetero atoms chosen from the group comprising N, O and/or S;
mono-, bi- or tricyclic heteroarylalkyl containing one, two, three or four hetero atoms chosen from the group comprising N, O and/or S, wherein the alkyl radical is a C₁-C₆ alkyl radical;
mono-, bi- or tricyclic heteroarylacyl containing one, two, three or four hetero atoms chosen from the group comprising N, O and/or S, wherein the acyl radical is a C₁-C₆-acyl radical and the heteroaryl radical can be substituted by one or more identical or different groups chosen from the group comprising halogen, C₁-C₆-alkyloxy, COO(C₁-C₆-alkyl), NH₂, NH(C₁-C₅-alkyl), N(C₁-C₅-alkyl)₂, CONH₂, CONH(C₁-C₆-alkyl), CON(C₁-C₆-alkyl)₂, OH, CF₃, CN, NO₂, and/or SO₂NH₂;
mono-, bi- or tricyclic (heteroaryl)alkenylacyl containing one, two, three or four hetero atoms chosen from the group comprising N, O and/or S, wherein the acyl radical is a C₁-C₆-acyl radical and the alkenyl radical is a C₂-C₆-alkenyl radical;
C(O)NH(C₁-C₁₀-alkyl); C(O)N(C₁-C₁₀-alkyl)₂, wherein the two alkyl radicals may form a saturated substituted or unsubstituted ring with the N atom; C(O)NH(aryl); C(O)NH(benzyl); C(O)(C₃-C₁₀-cycloalkyl); COO(aryl); COO(benzyl); COO(C₃-C₁₀-cycloalkyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0, 1, 2, 3 or 4; C(O)NH—(CH₂)ₖ—COO(C₁-C₆-alkyl), wherein k is 0, 1, 2, 3 or 4; C(O)NH—(CH₂)ₗ—CONH₂, wherein l is 0, 1, 2, 3 or 4;
COO—(CH₂)ₘ—COOH, wherein m is 0, 1, 2, 3 or 4; COO—(CH₂)ₘ—COO(C₁-C₁₀-alkyl), wherein n is 0, 1, 2, 3 or 4; COO—(CH₂)ₚ—C(O)NH₂, wherein p is 0, 1, 2, 3 or 4; C(O)—(CH₂)_{q}—COOH, wherein q is 0, 1, 2, 3 or 4; C(O)—(CH₂)ᵣ—COO(C₁-C₁₀-alkyl), wherein r is 0, 1, 2, 3 or 4; C(O)—(CH₂)ₛ—C(O)NH₂, wherein s is 0, 1, 2, 3 or 4; C(O)—(CH₂)ₜ—C(O)NH(C₁-C₆-alkyl), wherein t is 0, 1, 2, 3 or 4; C(O)—(CH₂)ᵤ—C(O)N(C₁-C₆-alkyl)₂, wherein u is 0, 1, 2, 3 or 4;
C(O)—(CH₂)ᵥ—NH₂, wherein v is 0, 1, 2, 3 or 4; C(O)—(CH₂)_{w}—OR′, wherein w is 0, 1, 2, 3 or 4 and R′ is H or C₁-C₆-acyl; C(O)—(CH₂)ₓ—C(O)NH—(CH₂)_{y}C(O)NH₂, wherein x is 0, 1, 2 or 3 and wherein y is 0, 1, 2 or 3;

SO₂(C₁-C₆-alkyl); SO₂—(CH₂)_{z}-heteroaryl, wherein z is 0, 1, 2 or 3; SO₂(CH₂)ₐ-heterocyclyl, wherein a is 0, 1, 2 or 3 and wherein the heterocyclyl residue may be substituted by one or more identical or different substituents chosen from the group comprising halogen, OH, CN, oxo and/or C₁-C₆-alkoxy; SO₂N(C₁-C₆-alkyl)₂ or SO₂NH(C₁-C₆-alkyl), wherein the alkyl radical can be substituted by halogen, C₁-C₄-alkoxy and/or OH; SO₂NH(C₃-C₆-cycloalkyl); SO₂NH—C(O)O(C₁-C₆-alkyl);

R², R³ are in each case identical or independent of each other and are chosen from the group comprising H; C₁-C₁₀-alkyl; C₃-C₁₀-cycloalkyl,
or
R² and R³ form, together with the nitrogen to which they are bonded, a saturated or unsaturated 3- to 8-membered N-heterocycle, wherein this can be substituted by one or more identical or different groups chosen from the group comprising halogen, OH, C₁-C₄-alkyloxy, COOH, COO(C₁-C₁₀-alkyl), CONH₂, CONH(C₁-C₁₀-alkyl), CON(C₁-C₁₀-alkyl)₂, CN, and/or O—C(O)(C₁-C₆ alkyl);

Z is chosen from the group comprising phenyl, which can be substituted by one or more identical or different groups chosen from the group comprising halogen, C₁-C₅-alkyl, C₁-C₅-alkoxy, NH₂, NH(C₁-C₅-alkyl), N(C₁-C₅-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl), wherein the substituents may form a ring;
a mono- or bicyclic aryl or heteroaryl containing one or two hetero atoms chosen from the group comprising N, O and/or S, wherein the aryl or heteroaryl group can be substituted by one or more identical or different groups chosen from the group comprising halogen, C₁-C₄-alkoxy, NH₂, NH(C₁-C₅-alkyl), N(C₁-C₅-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl).

The perhydroquinoxaline compounds of formula (1) according to the invention are named following the IUPAC nomenclature. In addition, the stereochemistry of the compounds of formula (1) follow the CIP nomenclature (Cahn-Ingold-Prelog) and may be specified as (4aR,5S,8aS) as long as the radical R¹ has the highest priority. Alternatively, if the priority under IUPAC of the C(O)CH₂Z moiety is higher than the one of R¹ the stereochemistry is defined as (4aS,8S,8aR). In the following general description, in the absence of any definition to the contrary, whenever the stereochemistry of the compounds of formula (1) in general is referred to, it is assumed that the radical R¹ has the highest priority and, thus, the (4aR,5S,8aS) definition applies. Consequently, the enantiomer of the compounds of formula (1) is referred to as the (4aS,5R,8aR) form.

It has been found, surprisingly, that the compounds according to the invention have an improved analgesic, antipruritic and antiinflammatory action. A particular advantage of the compounds according to the invention is the fact that the compounds have an analgesic action predominantly in the peripheral system.

Without wishing to be bound by a particular theory, it is assumed that not only the perhydroquinoxaline ring structure of the compounds according to the invention has a considerable influence on the advantageous properties of the compounds, but in particular the specific stereochemistry in the perhydroquinoxaline ring structure as shown in formula (1). In particular, the compounds according to the invention have been shown to act as κ opioid receptor agonists. This action is assumed to be responsible for the pharmaceutical efficacy.

One advantage of the compounds according to the invention is that they have a high affinity for the κ opioid receptor that is significantly higher than the affinity observed according to WO2009/080745. An advantage of a high selectivity of binding to the κ opioid receptor can be provided in that no or only mildly centrally mediated side effects occur. A particular advantage of a high selectivity of binding to the κ opioid receptor can be provided in that it is possible to reduce the risk of a psycho-dependency.

In the context of the present invention, unless stated otherwise, the term “heteroaryl” is to be understood as meaning mono-, bi- or tricyclic heteroaryl containing one, two, three or four hetero atoms chosen from the group comprising N, O and/or S.

Preferred heteroaryl radicals are chosen from the group comprising pyridinyl, pyrimidinyl, pyrazinyl, triazolyl, pyridazinyl, 1,3,5-triazinyl, quinolyl, isoquinolyl, quinolinyl, isoquinolinyl, quinoxalinyl, imidazolyl, pyrazolyl, benzimidazolyl, benzooxazolyl, benzothiazolyl, thiazolyl, oxazolyl, isoxazolyl, oxazolidinyl, pyrrolyl, carbazolyl, indolyl, isoindolyl, furyl, benzofuryl, benzofuranyl, 1,3-benzodioxolyl, thienyl and/or benzothienyl.

The term “C₁-C₁₀-alkyl” according to the invention includes, unless stated otherwise, straight-chain, branched or cyclic alkyl groups, preferably chosen from the group comprising methyl, ethyl, n-/i-propyl, n-/i-/tert-butyl, pentyl, neopentyl, hexyl, heptyl, octyl, nonyl and/or decyl.

The term “heterocyclyl” according to the invention includes saturated, mono- or diunsaturated cyclic alkyl radicals having 3 to 10 carbon atoms that contain one, two, three or four hetero atoms chosen from the group comprising NH, O and/or S.

C₁-C₆-alkoxy groups according to the invention are preferably chosen from the group comprising methoxy, ethoxy, linear or branched propoxy and/or butoxy.

The term “halogen” according to the invention includes fluorine, chlorine, bromine and iodine, fluorine or chlorine being preferred, in particular chlorine.

The term “aryl” according to the invention includes aromatic radicals having 6 to 20 carbon atoms, preferably phenyl, naphthyl, indenyl, and biphenyl. The term “aryl” also includes carbocycles.

In the context of the present invention, if not indicated otherwise, the term “acyl” means “C₁-C₁₀-acyl”, namely including the groups HC(O)— (formyl) and (C₁-C₉)—C(O)—, wherein (C₁-C₉) means linear, branched or cyclic alkyl or alkenyl groups. HC(O)— (formyl) and CH₃—C(O)— (acetyl) are preferred.

In preferred embodiments of the compounds of formula (1) the residues R¹, R², R³ and Z are as defined in the dependent claims 2 to 5.

Preferably in the compound according general formula (1)

R¹ is chosen from the group comprising H; C₁-C₃-alkyl; COO(C₁-C₄-alkyl);
benzyl;
C₁-C₄-acyl; C(O)C₄-C₆-cycloalkyl; heterocyclylacyl containing NH or O in the ring; phenylacyl, wherein the acyl radical is a C₁-acyl radical and the phenyl radical can be substituted by one or more identical or different groups chosen from the group comprising COO(C₁-C₃-alkyl) and CONH₂;
mono-cyclic heteroaryl containing one hetero atom chosen from the group of N, O and S;
mono-cyclic heteroarylalkyl containing one or two hetero atom chosen from the group of N, O and S, wherein the alkyl radical is a C₁-C₃ alkyl radical;
mono-cyclic heteroarylacyl containing one or two hetero atoms chosen from the group of N, O and S, wherein the acyl radical is a C₁-acyl radical and the heteroaryl radical can be substituted by one or more identical or different groups chosen from the group comprising COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals may form a saturated halogen substituted or unsubstituted ring with the N atom; C(O)NH(phenyl); C(O)NH(benzyl); C(O)(C₃-C₆-cycloalkyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)_{q}—COOH, wherein q is 0 or 1; C(O)—(CH₂)ᵣ—COO(C₁-C₃-alkyl), wherein r is 0 or 1; C(O)—(CH₂)ₛ—C(O)NH₂, wherein s is 0 or 1; C(O)—(CH₂)ₜ—C(O)NH(C₁-C₃-alkyl), wherein t is 0 or 1; C(O)—(CH₂)ᵤ—C(O)N(C₁-C₃-alkyl)₂, wherein u is 0 or 1;
C(O)—(CH₂)ᵥ—NH₂, wherein v is 0 or 1; C(O)—(CH₂)_{w}—OR′, wherein w is 0 or 1 and R′ is H or acetyl; C(O)—(CH₂)ₓ—C(O)NH—(CH₂)_{y}C(O)NH₂, wherein x is 0 or 1 and wherein y is 0 or 1;

SO₂(C₁-C₆-alkyl); SO₂—(CH₂)_{z}-heteroaryl, wherein z is 0 or 1; SO₂(CH₂)ₐ—heterocyclyl, wherein a is 0 or 1, wherein the heteroatoms are O, N, and/or S and wherein the heterocyclyl residue may be substituted by one or more identical or different substituents chosen from the group comprising F, Cl, OH, CN, oxo and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);

R², R³ are identical or different and are chosen from the group comprising H, methyl, ethyl, n-propyl, and i-propyl,
or
R² and R³ form, together with the nitrogen to which they are bonded, a saturated or mono-unsaturated 4- to 6-membered N-heterocycle, wherein this can be substituted by one or more identical or different groups chosen from the group comprising F, Cl, OH, CONH₂, CN, and/or O—C(O)(C₁-C₃ alkyl);

Z is chosen from the group comprising
phenyl, which can be substituted by one or more identical or different groups chosen from the group comprising F, Cl, C₁-C₃-alkyl, C₁-C₃-alkoxy, OH, CF₃, and NO₂, wherein two OH substituents may be connected by an ether bridge to form a ring or wherein two C₁-C₃-alkyl groups may be connected to form a saturated ring; and
a mono- or bicyclic aryl or heteroaryl containing one hetero atom chosen from the group of N and S, wherein the aryl or heteroaryl group can be substituted by one or more identical or different groups chosen from the group comprising F, Cl, C₁-C₃-alkyl, C₁-C₃-alkoxy, OH, CF₃, and NO₂.

More preferably in the compound according to general formula (1):

R¹ is chosen from the group consisting of
heterocyclylacyl containing NH or O in the ring; phenylacyl, wherein the acyl radical is a C₁-acyl radical and the phenyl radical is substituted by one or more of COO(C₁-C₃-alkyl) and CONH₂;
mono-cyclic heteroarylacyl containing one or two hetero atoms chosen from the group of N, O and S, wherein the acyl radical is a C₁-acyl radical and the heteroaryl radical is substituted by one or more of COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals form a saturated halogen substituted or unsubstituted ring with the N atom; C(O)NH(phenyl); C(O)NH(benzyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)ₛ—C(O)NH₂, wherein s is 0 or 1; C(O)—(CH₂)ₜ—C(O)NH(C₁-C₃-alkyl), wherein t is 0 or 1; C(O)—(CH₂)ᵤ—C(O)N(C₁-C₃-alkyl)₂, wherein u is 0 or 1;
C(O)—(CH₂)ᵥ—NH₂, wherein v is 1; C(O)—(CH₂)_{w}—OR′, wherein w is 1 and R′ is H or acetyl;
SO₂(C₁-C₆-alkyl); SO₂—(CH₂)_{z}-heteroaryl, wherein z is 0 or 1; SO₂(CH₂)ₐ-heterocyclyl, wherein a is 0 or 1, wherein the heteroatoms are O, N, and/or S and wherein the heterocyclyl residue may be substituted by one or more identical or different substituents chosen from the group comprising F, Cl, OH, CN, oxo and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);

R², R³ are identical or different and are chosen from the group comprising H, methyl, ethyl, n-propyl, and i-propyl,
or
R² and R³ form, together with the nitrogen to which they are bonded, a saturated or mono-unsaturated 4- to 6-membered N-heterocycle, wherein this can be substituted by one or more identical or different groups chosen from the group comprising F, Cl, OH, CONH₂, CN, and/or O—C(O)(C₁-C₃ alkyl);

Z is chosen from the group comprising
phenyl, which can be substituted by one or more identical or different groups chosen from the group comprising F, Cl, C₁-C₃-alkyl, C₁-C₃-alkoxy, OH, CF₃, and NO₂, wherein two OH substituents may be connected by an ether bridge to form a ring or wherein two C₁-C₃-alkyl groups may be connected to form a saturated ring; and
a mono- or bicyclic aryl or heteroaryl containing one hetero atom chosen from the group of N and S, wherein the aryl or heteroaryl group can be substituted by one or more identical or different groups chosen from the group comprising F, Cl, C₁-C₃-alkyl, C₁-C₃-alkoxy, OH, CF₃, and NO₂.

Particularly preferably in the compound according to general formula (1):

R¹ is chosen from the group comprising H; C₁-C₃-alkyl; COO(C₁-C₄-alkyl);
benzyl;
C₁-C₄-acyl; C(O)C₄-C₆-cycloalkyl; heterocyclylacyl containing NH or O in the ring; phenylacyl, wherein the acyl radical is a C₁-acyl radical and the phenyl radical can be substituted by one or more identical or different groups chosen from the group comprising COO(C₁-C₃-alkyl) and CONH₂;
mono-cyclic heteroaryl containing one hetero atom chosen from the group of N, O and S;
mono-cyclic heteroarylalkyl containing one or two hetero atom chosen from the group of N, O and S, wherein the alkyl radical is a C₁-C₃ alkyl radical;
mono-cyclic heteroarylacyl containing one or two hetero atoms chosen from the group of N, O and S, wherein the acyl radical is a C₁-acyl radical and the heteroaryl radical can be substituted by one or more identical or different groups chosen from the group comprising COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals may form a saturated halogen substituted or unsubstituted ring with the N atom; C(O)NH(phenyl); C(O)NH(benzyl); C(O)(C₃-C₆-cycloalkyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)_{q}—COOH, wherein q is 0 or 1; C(O)—(CH₂)ᵣ—COO(C₁-C₃-alkyl), wherein r is 0 or 1; C(O)—(CH₂)ₛ—C(O)NH₂, wherein s is 0 or 1; C(O)—(CH₂)ₜ—C(O)NH(C₁-C₃-alkyl), wherein t is 0 or 1; C(O)—(CH₂)ᵤ—C(O)N(C₁-C₃-alkyl)₂, wherein u is 0 or 1;
C(O)—(CH₂)ᵥ—NH₂, wherein v is 0 or 1; C(O)—(CH₂)_{w}—OR′, wherein w is 0 or 1 and R′ is H or acetyl; C(O)—(CH₂)ₓ—C(O)NH—(CH₂)_{y}C(O)NH₂, wherein x is 0 or 1 and wherein y is 0 or 1;
SO₂(C₁-C₆-alkyl); SO₂—(CH₂)_{z}-heteroaryl, wherein z is 0 or 1; SO₂(CH₂)ₐ-heterocyclyl, wherein a is 0 or 1, wherein the heteroatoms are O, N, and/or S and wherein the heterocyclyl residue may be substituted by one or more identical or different substituents chosen from the group comprising F, Cl, OH, CN, oxo and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);
R² and R³ form, together with the nitrogen to which they are bonded, a mono-unsaturated 6-membered N-heterocycle, that may be substituted by one or more of F, Cl, OH, CONH₂, CN, and/or O—C(O)(C₁-C₃ alkyl);

Z is chosen from the group comprising
phenyl, which can be substituted by one or more identical or different groups chosen from the group comprising F, Cl, C₁-C₃-alkyl, C₁-C₃-alkoxy, OH, CF₃, and NO₂, wherein two OH substituents may be connected by an ether bridge to form a ring or wherein two C₁-C₃-alkyl groups may be connected to form a saturated ring; and
a mono- or bicyclic aryl or heteroaryl containing one hetero atom chosen from the group of N and S, wherein the aryl or heteroaryl group can be substituted by one or more identical or different groups chosen from the group comprising F, Cl, C₁-C₃-alkyl, C₁-C₃-alkoxy, OH, CF₃, and NO₂.

Particularly preferably in the compound according to general formula (1):

R¹ is chosen from the group comprising H; C₁-C₃-alkyl; COO(C₁-C₄-alkyl);
benzyl;
C₁-C₄-acyl; C(O)C₄-C₆-cycloalkyl; heterocyclylacyl containing NH or O in the ring; phenylacyl, wherein the acyl radical is a C₁-acyl radical and the phenyl radical can be substituted by one or more identical or different groups chosen from the group comprising COO(C₁-C₃-alkyl) and CONH₂;
mono-cyclic heteroaryl containing one hetero atom chosen from the group of N, O and S;
mono-cyclic heteroarylalkyl containing one or two hetero atom chosen from the group of N, O and S, wherein the alkyl radical is a C₁-C₃ alkyl radical;
mono-cyclic heteroarylacyl containing one or two hetero atoms chosen from the group of N, O and S, wherein the acyl radical is a C₁-acyl radical and the heteroaryl radical can be substituted by one or more identical or different groups chosen from the group comprising COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals may form a saturated halogen substituted or unsubstituted ring with the N atom; C(O)NH(phenyl); C(O)NH(benzyl); C(O)(C₃-C₆-cycloalkyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)_{q}—COOH, wherein q is 0 or 1; C(O)—(CH₂)ᵣ—COO(C₁-C₃-alkyl), wherein r is 0 or 1; C(O)—(CH₂)ₛ—C(O)NH₂, wherein s is 0 or 1; C(O)—(CH₂)ₜ—C(O)NH(C₁-C₃-alkyl), wherein t is 0 or 1; C(O)—(CH₂ᵤ—C(O)N(C₁-C₃-alkyl)₂, wherein u is 0 or 1;
C(O)—(CH₂)ᵥ—NH₂, wherein v is 0 or 1; C(O)—(CH₂)_{w}—OR′, wherein w is 0 or 1 and R′ is H or acetyl; C(O)—(CH₂)ₓ—C(O)NH—(CH₂)_{y}C(O)NH₂, wherein x is 0 or 1 and wherein y is 0 or 1;
SO₂(C₁-C₆-alkyl); SO₂—(CH₂)_{z}-heteroaryl, wherein z is 0 or 1; SO₂(CH₂)ₐ-heterocyclyl, wherein a is 0 or 1, wherein the heteroatoms are O, N, and/or S and wherein the heterocyclyl residue may be substituted by one or more identical or different substituents chosen from the group comprising F, Cl, OH, CN, oxo and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);

R², R³ are identical or different and are chosen from the group comprising H, methyl, ethyl, n-propyl, and i-propyl,
or
R² and R³ form, together with the nitrogen to which they are bonded, a saturated or mono-unsaturated 4- to 6-membered N-heterocycle, wherein this can be substituted by one or more identical or different groups chosen from the group comprising F, Cl, OH, CONH₂, CN, and/or O—C(O)(C₁-C₃ alkyl);

Z is either a tetrahydronaphthyl or a 2,3-dihydrobenzo-1,4-dioxinyl residue, optionally substituted by one or more of F, Cl, C₁-C₃-alkyl, C₁-C₃-alkoxy, OH, CF₃, and NO₂.

Particularly preferred radicals R¹ according to the invention are as follows:

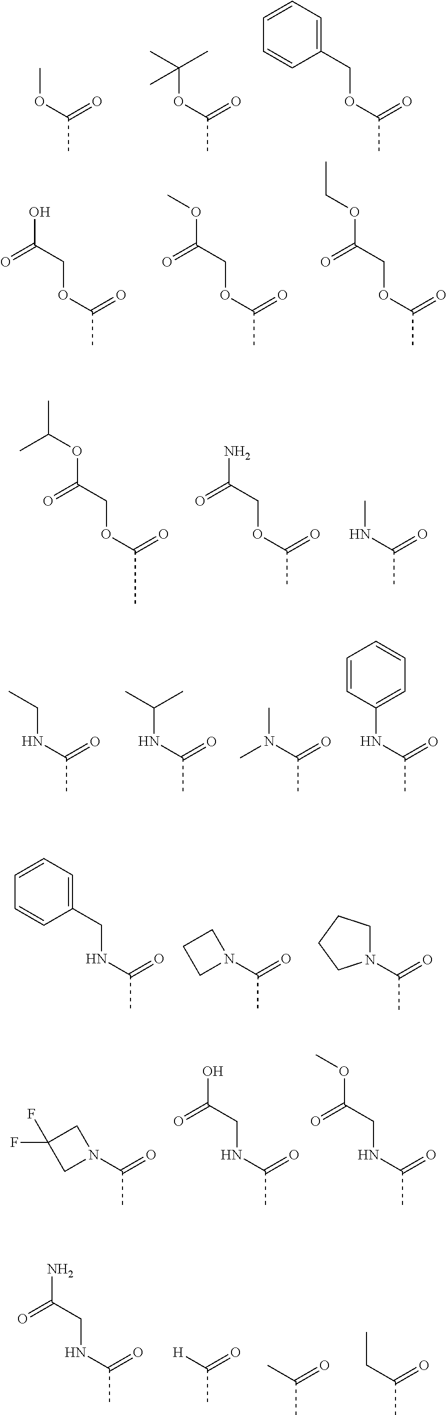

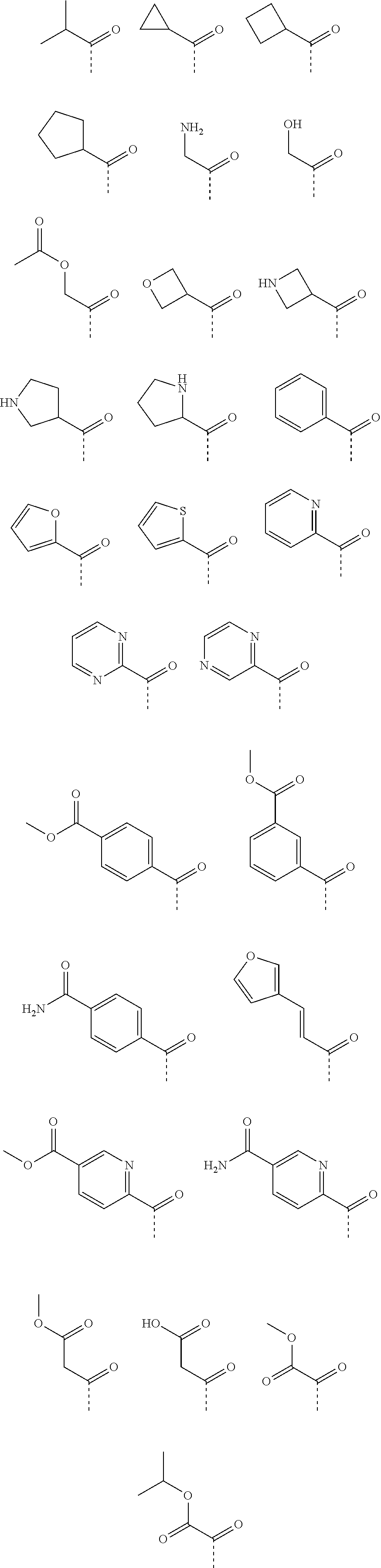

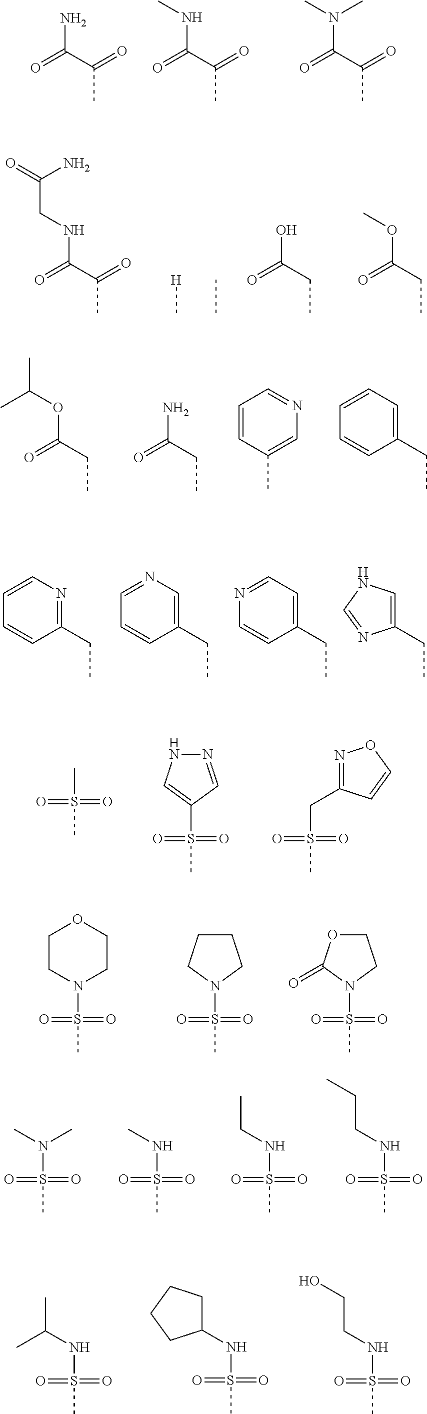

Particularly preferred radicals NR²R³ according to the invention are as follows:

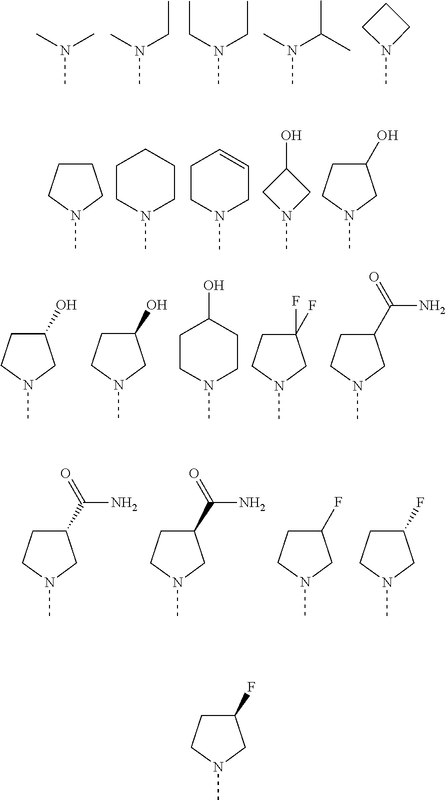

Particularly preferred radicals Z according to the invention are as follows:

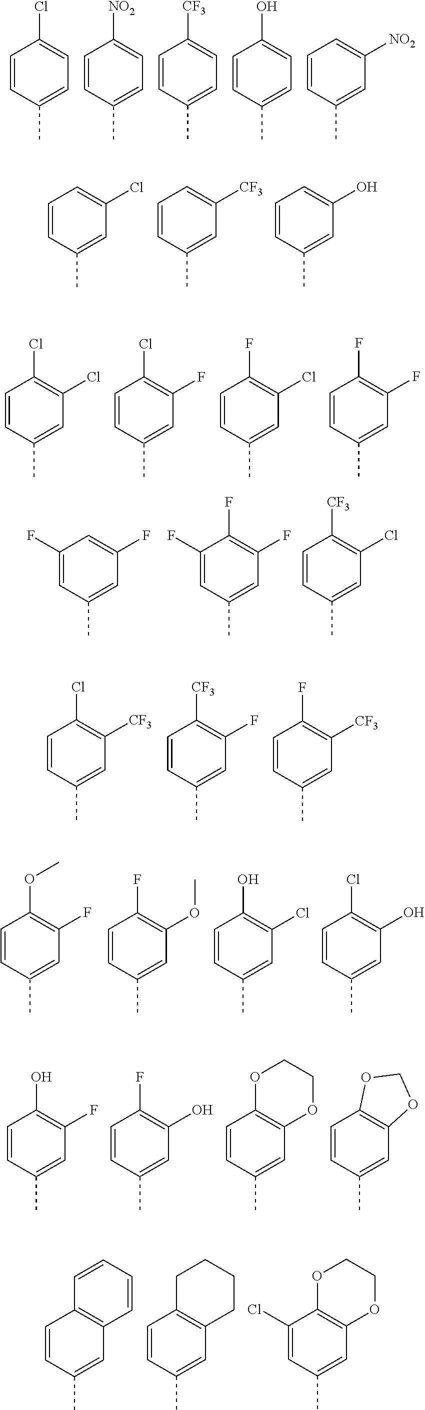

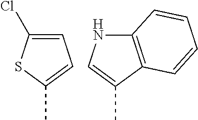

Without being bound by a particular theory, it is assumed that the action of the compounds according to the invention is not only based on the steric action of the perhydroquinoxaline group, in particular in combination with the structural element R¹, but even more on the specific cis-trans stereochemistry and the (4aR,5S,8aS) form of the compounds as indicated in formula (1). Reference is made to the Biological Assay section of the application.

The compounds according to the invention can furthermore be used in the form of their acids or their bases or in the form of their salts, in particular the physiologically acceptable salts, or in the form of their solvates, in particular their hydrates.

The pharmaceutically acceptable salts can be base addition salts. These include salts of the compounds according to the invention with inorganic bases, such as alkali metal hydroxides, alkaline earth metal hydroxides, or with organic bases, such as mono-, di- or triethanolamine

Acid addition salts, in particular with inorganic acids, such as hydrochloric acid, sulfuric acid or phosphoric acid, or with suitable organic carboxylic or sulfonic acids, or with amino acids, can further advantageously be used.

Pharmaceutically acceptable salts of the compounds according to the invention are chosen, for example, from the group comprising chlorides, bromides, iodides, hydrochlorides, hydrobromides, sulfonates, methanesulfonates, sulfates, hydrogen sulfates, sulfites, hydrogen sulfites, phosphates, nitrates, methanoates, acetates, proprionates, lactates, citrates, glutarates, maleates, malonates, malates, succinates, tartrates, oxalates, fumarates, benzoates, p-toluenesulfonates and/or salts of amino acids, preferably the proteinogenic amino acids.

The compounds according to the invention are suitable for use as medicaments. They are capable of having an analgesic, antipyretic, antipruritic, antiinflammatory and/or spasmolytic action.

In preferred embodiments, one advantage of the compounds is that these compounds pass the blood-brain barrier to only a small extent. This makes it possible for the compounds according to the invention to be usable in particular as peripherally acting analgesics and anti-inflammatory agents.

In advantageous embodiments the compounds according to the invention can be used in particular for therapeutic and/or prophylactic treatment, diagnosis and/or therapy of diseases chosen from the group comprising pain- or pruritus-related diseases and/or inflammatory diseases.

The invention also provides the use of the compounds according to the invention for the preparation of a medicament for therapeutic and/or prophylactic treatment of diseases chosen from the group comprising pain- or pruritus-related diseases, and/or inflammatory diseases.

The compounds according to the invention can be used by themselves or in combination with known substances for treatment of diseases chosen from the group comprising pain- or pruritus-related diseases, and/or inflammatory diseases. Preferably the compounds of the invention are used as peripheral analgesics or antiinflammatory agents.

Pain-related diseases are chosen from the group comprising back pain, facial pain, headaches, migraine, joint pain, muscular pain syndromes, inflammatory pain-related diseases, neuropathic pain, peripheral pain, peripheral nerve damage, visceral pain, abdominal pain, menstruation symptoms, kidney- and gallstone pain, pruritus, cancer and tumor pain, sympathetic pain, postoperative pain, postraumatic pain, hyperalgesia and/or inflammatory pain.

Inflammatory diseases are chosen from the group comprising inflammatory diseases of the gastrointestinal tract, in particular inflammatory bowel diseases, such as Crohn's disease and/or colitis ulcerosa, acute or chronic inflammatory changes with inflammation of the gall bladder, inflammatory pseudopolyps, colitis cystica profunda, pneumatosis cystoides intestinales, pancreatitis, appendicitis, cardiovascular inflammation due to arthereosclerosis, ischemia, restenosis and/or vasculitis, sepsis, septicemia, allergies, asthma, Sjogren's syndrome, pulmonary inflammation, chronic airway inflammation, chronic obstructive pulmonary disease (COPD), tumor proliferation, tumor metastasis, transplant rejection, inflammatory diseases of the joints, such as rheumatoid arthritis, vulvovaginitis (all causes), and/or inflammatory diseases of the brain, skin, hair follicle, urogenital tract and of the eyes. Further inflammatory diseases comprise sinusitis, tenosynovitis, bursitis, tendonitis, lateral epicondylitis, adhesive capsulitis, osteomyelitis, osteoarthritic inflammation, ocular inflammation, otitic inflammation and autoimmune inflammation.

Pruritus (itching) is a frequent symptom in skin therapy conventionally experienced as a type of pain stimulus. The itching sensation triggers the desire to scratch the affected area. Skin damaged by scratching further offers infectious pathogens a good nutrient medium and inflammations of scratched-open areas of skin are not infrequent. Pruritic skin and hair diseases are chosen from the group comprising pruritus, psoriasis, psoriatic arthritis, contact dermatitis, atopic eczema, scleroderma and other fibrotic diseases, systemic lupus erythematous, urticaria, lichen planus, lymphoma and/or allergic diseases or characterized by mast cell involvements.

The diseases in the sense of the present invention also comprise other diseases such as hyponatremia, edema, ileus, tussis, glaucoma, MS (multiple sclerosis), Morbus Parkinson and Morbus Alzheimer.

The organs involved in the pain- or pruritus-related diseases and/or inflammatory diseases are in particular the so-called barrier organs, namely the gastrointestinal tract, skin, lung, urogenital tract; the brain; the ear nose and throat tract; teeth; bones; liver; and hair. Particularly preferred embodiments of the invention relate to the treatment of the diseases of the barrier organs.

Diseases of the gastrointestinal tract are chosen from the group comprising irritable bowel syndrome, gastric lesions, gastrointestinal ulcerations, exogenous and endogenous damage to the gastrointestinal mucosa, malfunctions of the gastrointestinal tract, adenomas, in particular in the intestine, and/or juvenile polyps.

Diseases of the lung (respiratory diseases) include inflammatory lung disease, obstructive lung diseases such as chronic obstructive pulmonary disease (COPD), restrictive lung diseases, respiratory tract infections such as upper respiratory tract infection, lower respiratory tract infection, malignant tumors and benign tumors, pleural cavity diseases, pulmonary vascular diseases, and neonatal diseases.

Diseases of the urogenital tract include analgesic nephropathy, bladder cancer, cystocele (fallen bladder), end stage renal disease (ESRD), glomerulonephritis, glomerulosclerosis, goodpasture syndrome, hematuria (blood in the urine), hemolytic uremic syndrome, immunoglobulin A (IgA) nephropathy, impotence/erectile dysfunction, interstitial cystitis, kidney cancer, kidney stones, kidney transplantation, male factor infertility, nephrotic syndrome, neurogenic bladder, Peyronie's disease, and polycystic kidney disease.

Further diseases that may be treated with the compounds of the present invention are described in US 2011/0212882 A1 being incorporated herein by reference.

A further advantage of the compounds according to the invention results from the fact that no or only mildly centrally mediated side effects, such as respiratory depression, vomiting, bradycardia or constipation, may occur.

It is of particular advantage that the compounds according to the invention preferably show no euphoric action. Thus, the administration of the compounds according to the invention lead to relatively mild or no psycho-dependency. This makes it possible to be able to administer the compounds according to the invention over a relatively long period of time. For example, a long-term administration, in particular a daily administration, is made possible.

The compounds according to the invention can furthermore be suitable as a local anesthetic. For example, the compounds according to the invention can be suitable for alleviating the pain of insect bites, such as mosquito bites, or burns.

The compounds according to the invention or compositions containing these can be administered systemically or topically. Preferably, the compounds or compositions according to the invention are administered topically, in particular in the form of creams, ointments, plasters or tinctures.

In the context of the present invention, the term “prophylactic treatment” is understood as meaning in particular that the compounds according to the invention can be administered before symptoms of a disease occur or the risk of a disease exists.

The medicaments according to the invention may further comprise at least one opioid receptor antagonist, preferably chosen from the group comprising naloxone, naltrexone, cyprodime, naltrindole, norbinaltorphimine nalmefene, nalorphine, nalbuphine, naloxonazine, methylnaltrexone and/or ketylcyclazocine, and/or a steroidal anti-inflammatory drug, preferably chosen from the group of hydrocortisone, hydrocortisone acetate, prednisolone, methylprednisolone, prednisone, betamethasone, hydrocortisone-17-valerate, betamethasone valerate, betamethasone dipropionate, prednicarbate, clobetasone-17-butyrate flunisolide, fluticasone propionate, triamcinolone acetonide, beclomethasone dipropionate, budesonide and/or hydrocortisone-17-butyrate and/or a nonsteroidal anti-inflammatory drug (NSAID), preferably chosen from the group of aspirin, ibuprofen, diclofenac and/or naproxen, and/or an opioid receptor agonist, preferably chosen from the group comprising tramadol, pethidin, codein, piritramid, morphin, levomethadon, fentanyl, alfentanil, remifentanil and/or sufentanil, and/or an antibiotic.

The compounds according to the invention can be administered according to conventional methods, for example orally, dermally, intranasally, transmucosally, pulmonally, enterally, buccally, rectally, intraurethral, aural, by inhalation, by means of injection, for example intravenously, parenterally, intraperitoneally, intradermally, subcutaneously and/or intramuscularly and/or locally, for example on painful areas of the body. Oral administration is particularly preferred.

The compounds according to the invention can be used in particular for the preparation of medicaments by being brought into a suitable dosage form together with at least one carrier substance or auxiliary substance, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules.

Pharmaceutical dosage forms with delayed release (sustained release formulation) are furthermore preferred for oral administration of the compounds according to the invention. Examples of formulations with delayed release are sustained release matrix tablets, multilayered tablets, the coating of which can be, for example, constructed to be resistant to gastric juice, such as coatings based on shellac, sustained release capsules or formulations using biodegradable polymers, for example poly(lactic acid) polymers.

Conventional physiologically acceptable pharmaceutical auxiliary substances, preferably chosen from the group comprising carrier materials, fillers, solvents, diluents, wetting agents, emulsifiers, dyestuffs, preservatives, disintegrating agents, lubricants, salts for influencing the osmotic pressure, buffer substances, aromas and/or binders, can be used for the preparation of the medicaments.

The compounds according to the invention can be prepared by a process comprising the following steps:
a) reacting 5,6,7,8-tetrahydroquinoxalin-5-ol with a protection agent X-PG in the presence of a base to introduce a protecting group PG at the alcohol function, wherein X is a suitable leaving group;
b) catalytically hydrogenating the PG protected 5,6,7,8-tetrahydroquinoxalin-5-ol obtained in step a) under stereoselective reduction of the pyrazine ring to obtain PG protected cis-cis 5-hydroxy-decahydroquinoxaline;
c) reacting the PG protected cis-cis 5-hydroxy-decahydroquinoxaline obtained in step b) with a reagent X—R¹ to regioselectively introduce the substituent R¹ at the 1-N atom of the cis-cis 5-hydroxy-decahydroquinoxaline, wherein X is a suitable leaving group;
d) deprotecting the PG protected hydroxy group in the product obtained in step c) to provide for the corresponding α,β-aminoalcohol;
e) reacting the α,β-aminoalcohol obtained in step d) with sulfuryl chloride in the presence of a base to provide for the corresponding 1,2,3-oxathiazolidine 2,2-dioxide;
f) reacting the 1,2,3-oxathiazolidine 2,2-dioxide obtained in step e) with an amine HNR²R³, followed by treatment with an acid to introduce the residue —NR²R³ under inversion of the stereogenic center to provide for cis,trans 5-amino-octahydroquinoxaline; and
g) reacting the cis,trans 5-amino-octahydroquinoxaline obtained in step f) with an activated carboxylic acid derivative ZCH₂COY, wherein Y is a suitable leaving group, preferably with an acid chloride Z-CH₂COCl, under acylation in 4-position to provide for the compound of formula (1).

By this reaction (shown in Reaction Scheme 10 below in more detail) a racemate comprising two enantiomers is formed, namely next to the (4aR,5S,8aS) form of formula (1) also the enantiomeric (4aS,5R,8aR) form is obtained. In a preferred embodiment of the invention the process further comprises the step of separating the compound of formula (1) from its enantiomeric (4aS,5R,8aR) form.

The separation of the enantiomers can be carried out by known methods, in particular chromatography methods, preferably by means of high performance liquid chromatography (HPLC) or column chromatography or flash chromatography (FC), even more preferably by chiral chromatography methods, in particular chiral high performance liquid chromatography.

The separation of the enantiomers can also be carried out by reaction of a racemic mixture of an organic acid with a pure enantiomer of an acid. The diastereomeric salts formed can be separated by fractional crystallization. The splitting of the racemate is preferably carried out by reacting the racemate with an enantiomerically pure acid. The separation is then carried out by fractional recrystallization or chromatography methods, it being possible for the methods to be combined and carried out several times.

The compound of formula (1) may be obtained in enantiomerically pure (4aR,5S,8aS) form by the process described above when subjecting enantiomerically pure (R)-5,6,7,8-tetrahydroquinoxalin-5-ol to the reaction steps a) to g). (R)-5,6,7,8-tetrahydroquinoxalin-5-ol may be obtained according to the invention by
(a1) oxidizing 5,6,7,8-tetrahydroquinoxalin-5-ol to the corresponding ketone with an oxidizing agent;
(a2) subjecting the ketone obtained in step (a1) to an asymmetric hydrogen transfer reaction using a hydrogenation agent and a chiral catalyst to provide for enantiomerically pure (R)-5,6,7,8-tetrahydroquinoxalin-5-ol.

This reaction is shown in Reaction Scheme 11 below in more detail.

As the chiral catalyst dichloro(p-cymene)ruthenium(II) dimer with enantiomeric (1R,2R)-N-p-tosyl-1,2-diphenylethylenediamine or enantiomeric (S)-Me-CBS-oxazoborolidine as the ligand may be used

Finally, the compounds of formula (1) obtained may be converted to pharmaceutically acceptable salts by reaction with the corresponding acid in a common way.

In the following the preparation of the compounds of formula (1) according to the present invention and of related reference compounds is described in more detail.

In the schemes, preparations and examples below, various reagent symbols and abbreviations have the following meanings:

Alloc allyloxycarbonyl

Boc tert-butoxycarbonyl

Bn benzyl

Cbz benzyloxycarbonyl

DCM dichloromethane

DIEA ethyl-diisopropylamine

DMAP 4-dimethylaminopyridine

DMF N,N-dimethylformamide

DMS dimethylsulfide

DMSO dimethyl sulfoxide

ee enantiomeric excess

Et₂O diethyl ether

EtOAc ethyl acetate

EtOH ethanol

h hour(s)

HOAc acetic acid

m/z mass-to-charge ratio

mCPBA 3-chloroperbenzoic acid

min minute(s)

NBS N-bromosuccinimide

MeCN acetonitrile

MeOH methanol

mp melting point

MW molecular weight

PG protecting group

Ph phenyl

RT room temperature

T temperature

TBDMS tert-butyldimethylsiliyl

TEA triethylamine

TFA trifluoroacetic acid

TFAA trifluoroacetic acid anhydride

THF tetrahydrofuran

TLC thin layer chromatography

t_{R} (min) HPLC retention time

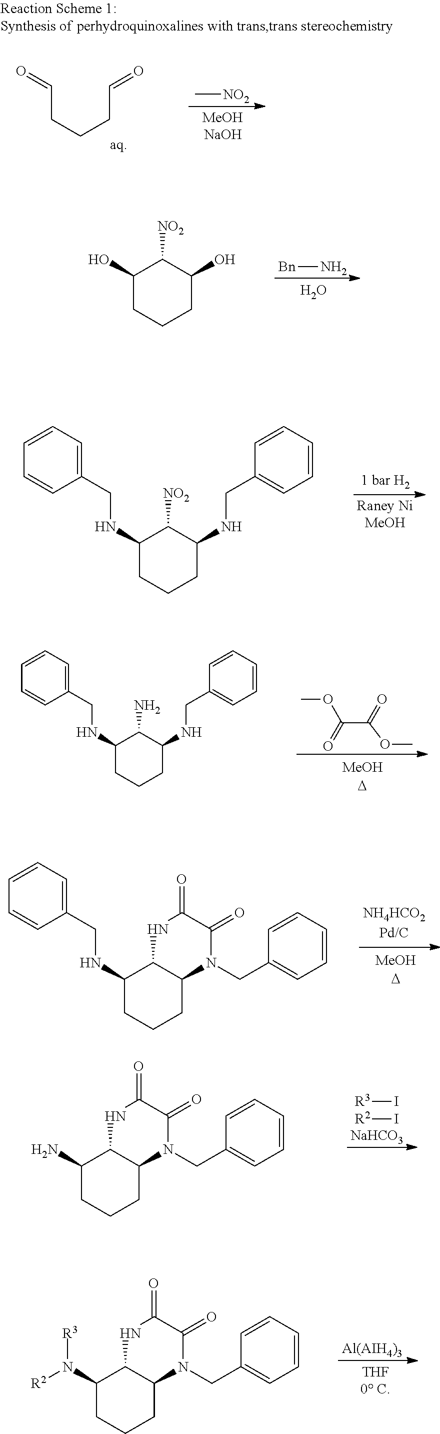

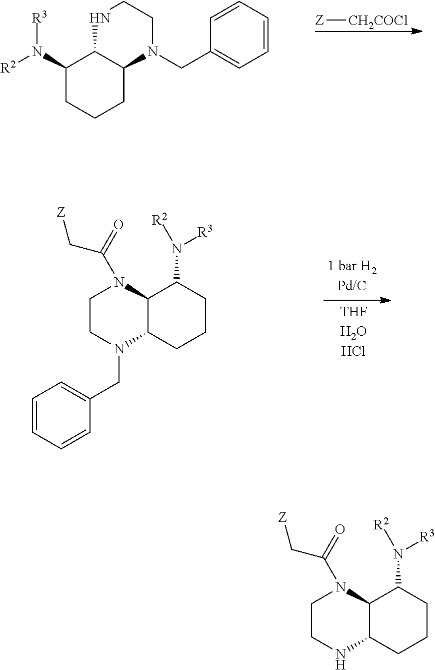

Optionally substituted perhydroquinoxalines with trans,trans stereochemistry can be obtained as shown in Reaction Scheme 1. Aqueous glutaraldehyde can be reacted with nitromethane in a double Henry reaction to the cyclic nitrodiol in a solvent like methanol using a catalyst such as sodium hydroxide. Reaction with benzylamine in water provides the nitrodiamine which can subsequently be reduced to the cyclohexanetriamine in a suitable solvent like methanol with hydrogen under Raney nickel catalysis. Reaction with dimethyl oxalate in a solvent such as methanol under reflux conditions provides the quinoxalindione. Selective debenzylation of the exocyclic amine can be achieved by reaction with ammonium formate and palladium on charcoal in a solvent like methanol under reflux conditions. Residues R² and R³ can be introduced by means of an alkylation reaction in a solvent like MeCN in the presence of a base such as NaHCO₃ at elevated temperature. Reagents like for example methyl iodide or ethyl iodide can be used for synthesis of compounds in which R² is equal to R³. Compounds in which R² and R³ form, together with the nitrogen to which they are bonded, a saturated 3- to to 8-membered N-heterocycle can be obtained applying optionally substituted alkylendihalogenides such as 1,4-diiodobutane, 1,4-dibromo-2-hydroxybutane and 1,5-diiodopentane. Reduction with aluminium tri(tetrahydridoaluminate) in an inert solvent like THF at low temperature leads to optionally substituted perhydroquinoxalines. Substituents Z—CH₂CO can be introduced by reaction with the corresponding acid chloride in a solvent such as DCM. Finally, the second benzyl protecting group can be removed under catalytic hydrogenation conditions. Substituents R¹ can be introduced as described in Reaction Scheme 8.

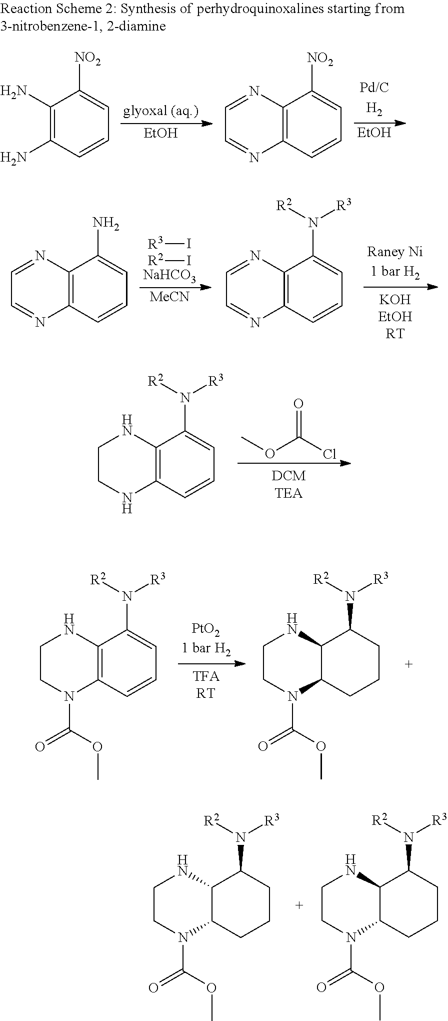

As depicted in Reaction Scheme 2, cyclization of 3-nitrobenzen-1,2-diamine with aqueous glyoxal in ethanol yields 5-nitroquinoxaline which can subsequently be hydrogenated in the presence of a catalyst like palladium on charcoal in a solvent such as ethanol. 5-Aminoquinoxazoline thus obtained can be alkylated with for example methyl iodide or ethyl iodide for synthesis of compounds in which R² is equal to R³. Compounds in which R² and R³ form, together with the nitrogen to which they are bonded, a saturated 3- to 8-membered N-heterocycle can be obtained applying optionally substituted alkylendihalogenides such as 1,4-diiodobutane, 1,4-dibromo-2-hydroxybutane and 1,5-diiodopentane. Alkylation reactions can be performed in a solvent such as MeCN in the presence of a base like NaHCO₃. Selective hydrogenation in the presence of a catalyst like Raney nickel and a base such as potassium hydroxide in a solvent like ethanol yields optionally substituted 1,2,3,4-tetrahydroquinoxalin-5-amine which can be reacted stereoselectively with methyl chloroformate in a solvent like DCM in presence of a base such as TEA. Subsequently, the phenyl ring can be hydrogenated in the presence of a catalyst such as for example PtO₂ in a solvent like trifluoroacetic acid. The perhydroquinoxazolines are obtained as a mixture of three diastereomers. The cis,cis isomer can be isolated directly after column chromatography. The other two isomers (trans,cis and cis,trans) are separated after the acylation with Z—CH₂COCl (see reaction Scheme 3).

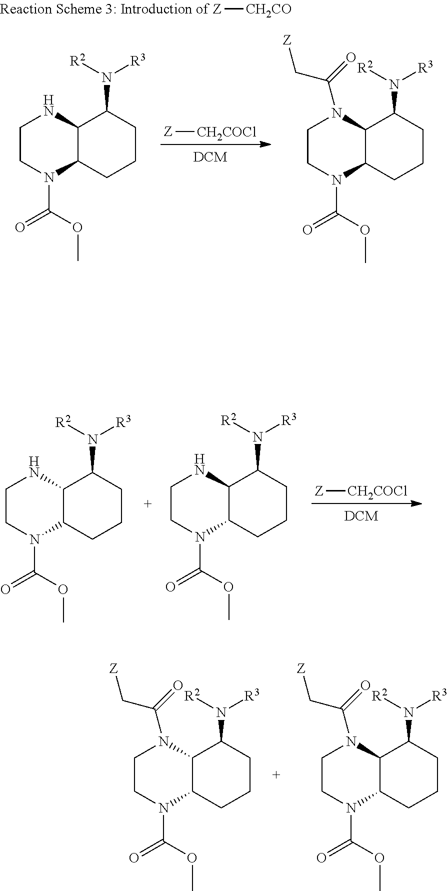

Optionally substituted methyl 5-aminooctahydroquinoxaline-1(2H)-carboxylate can be acylated in 4-position with acid chlorides Z—CH₂COCl in a solvent like DCM with or without the presence of a base such as DIEA.

When a mixture of trans,cis and cis,trans isomers is used as starting material the diastereomeric products can be separated following the acylation step.

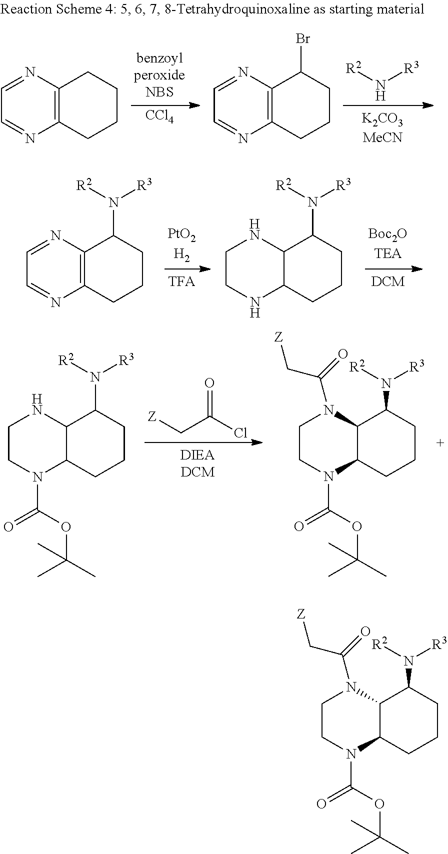

An alternative reaction pathway leading to optionally substituted perhydroquinoxazolines is shown in Reaction Scheme 4. 5,6,7,8-Tetrahydroquinoxaline can be brominated in benzylic position with NBS and benzoyl peroxide in an inert solvent like tetrachloromethane. Subsequent reaction with amines HNR²R³ in a solvent like MeCN in the presence of a base such as potassium carbonate yields optionally substituted 5,6,7,8-tetrahydroquinoxalin-5-amine Hydrogenation of the pyrazine ring can be accomplished in the presence of a catalyst like PtO₂ in a solvent like trifluoroacetic acid. The perhydroquinoxazoline thus obtained can be selectively Boc-protected in 1-position with Boc2O in a solvent such as DCM in the presence of a base like TEA. Acylation in 4-position with acid chlorides Z—CH₂COCl in a solvent like DCM with or without the presence of a base such as DIEA yields the cis,cis and the trans,trans isomers which can be separated by column chromatography.

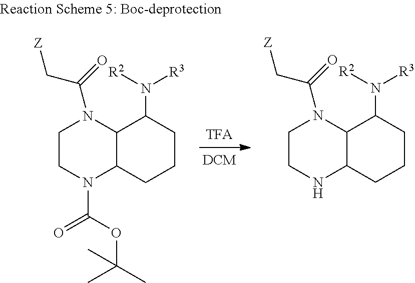

Optionally substituted Boc-protected perhydroquinoxazoline can be deprotected with trifluoroacetic acid in DCM. Alternatively, reagents such as HCl in suitable solvents like dioxane, diethyl ether and THF may be applied.

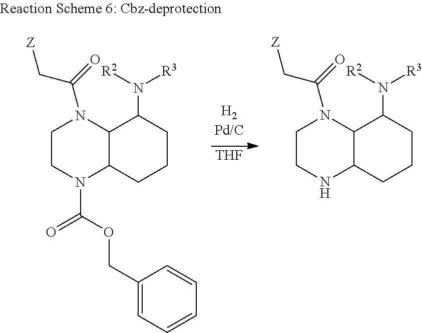

Optionally substituted Cbz-protected perhydroquinoxazoline can be deprotected by hydrogenation in the presence of a catalyst such as palladium on charcoal in the presence in a suitable solvent like a THF or ethyl acetate. Alternatively, the unprotected compound can be obtained by reaction with an acid like trifluoroacetic acid in the presence of a reagent such as thioanisole.

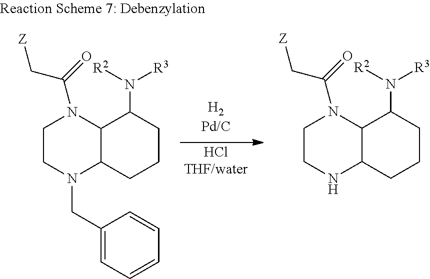

Optionally substituted benzyl-protected perhydroquinoxazoline can be deprotected by hydrogenation in the presence of a catalyst such as palladium on charcoal in the presence in a suitable solvent like a mixture of THF and aqueous hydrochloric acid.

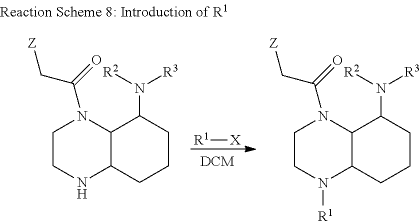

Optionally substituted [8-amino octahydroquinoxalin-1(2H)-yl]ethanones obtained as described in Reaction Schemes 1 and 5-7 can be reacted with various reagents for introduction of R¹ as shown in Reaction Scheme 8.

Reaction with optionally substituted acid chlorides in an inert solvent like DCM with or without a base yields compounds wherein R¹ is chosen from C₁-C₁₀-acyl, C₃-C₁₀-cycloacyl, phenylacyl, heteroarylacyl, C(O)COO(C₁-C₁₀-alkyl) and C(O)—(CH₂)ᵣ—COO(C₁-C₁₀-alkyl). Residues C(O)—(CH₂)ᵣ—COOH can be introduced by reaction with cyclic acid anhydrides in an inert solvent like DCM in the presence of a catalyst such as DMAP.

Carbamates in which R¹ is selected from COO(C₁-C₁₀-alkyl), COO(aryl) and COO(C₃-C₁₀-cycloalkyl) can be obtained by reacting the starting material with the corresponding optionally substituted alkyl-, aryl- and cycloalkylchloroformates in an inert solvent such as DCM. Reaction of optionally substituted [8-aminooctahydroquinoxalin-1(2H)-yl]ethanones with optionally substituted carbamoyl chlorides in a solvent such as DCM yields ureas C(O)NH(C₁-C₁₀-alkyl) and C(O)N(C₁-C₁₀-alkyl)₂. Alternatively, ureas in which R¹ is C(O)NH(C₁-C₁₀-alkyl) can also be obtained using the corresponding isocyanates. Compounds in which R¹ represents C₁-C₁₀-alkyl, phenylalkyl and heteroarylalkyl can be obtained using two different methodologies. The corresponding optionally substituted aldehydes can be subjected to a reductive amination reaction with optionally substituted [8-aminooctahydroquinoxalin-1(2H)-yl]ethanones to yield the alkylated compounds. The reaction is performed in a suitable solvent like MeOH in the presence of a reducing agent like NaBH₃CN with pH adjustment by concentrated acetic acid. Alternatively, above mentioned residues can also be introduced in an alkylation reaction using appropriate optionally substituted C₁-C₁₀-alkylhalogenides, C₃-C₁₀-cycloalkylhalogenides, phenylalkylhalogenides and heteroarylalkylhalogenides. Alkylation reactions can be conducted in a solvent like MeCN in the presence of a base such as NaHCO₃ or in a solvent like DCM or chloroform in the presence of a base such as DIEA.

Reaction with optionally substituted sulfonyl chlorides in an inert solvent like DCM with or without a base yields compounds wherein R¹ is chosen from SO₂(C₁-C₆-alkyl), SO₂—(CH₂)_{z}-heteroaryl and SO₂(CH₂)ₐ-heterocyclyl, respectively.

Reaction of optionally substituted [8-aminooctahydroquinoxalin-1(2H)-yl]ethanones with optionally substituted sulfamoyl chlorides in a solvent such as DCM with or without a base yields compounds wherein R¹ is chosen from SO₂N(C₁-C₆-alkyl)₂, SO₂NH(C₁-C₆-alkyl), SO₂NH(C₃-C₆-cycloalkyl) and SO₂NH—C(O)O(C₁-C₆-alkyl), respectively.

If NR²R³ contains functional groups, these can be protected before R¹ is introduced and deprotected in a subsequent reaction step. A hydroxyl group for example can be protected as acetate.

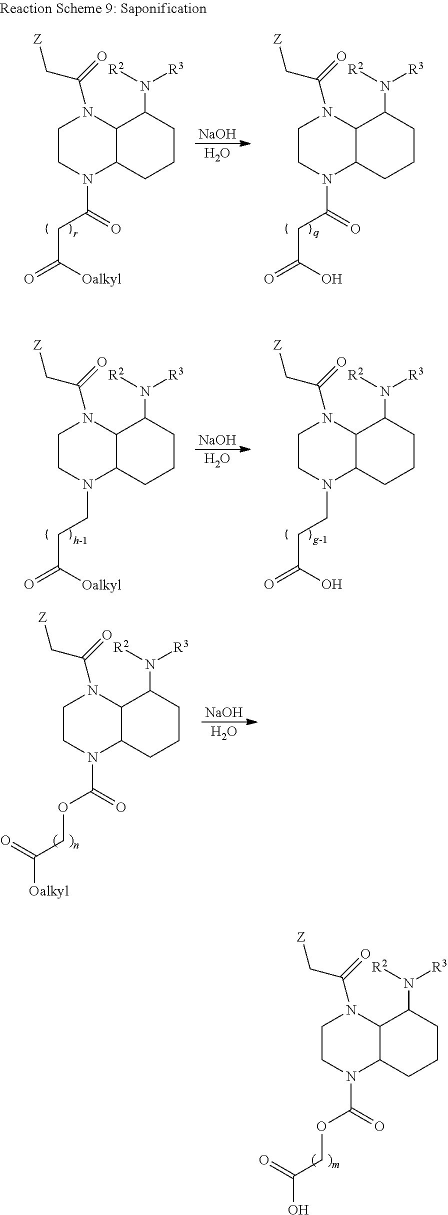

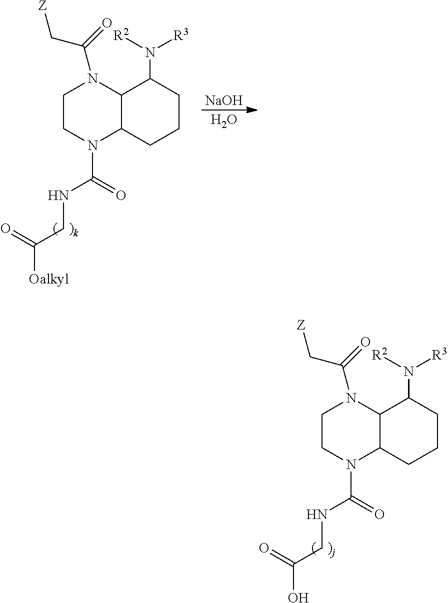

Optionally substituted perhydroquinoxazolines in which R¹ is C(O)—(CH₂)ᵣ—COO(C₁-C₆-alkyl), C(O)(CH₂)ₕCOO(C₁-C₆-alkyl), COO—(CH₂)ₙ—COO(C₁-C₁₀-alkyl) and C(O)NH—(CH₂)ₖ—COO(C₁-C₆-alkyl), respectively, can be transferred to the corresponding acids by reaction with a base such as sodium hydroxide in a solvent like water as shown in Reaction Scheme 9.

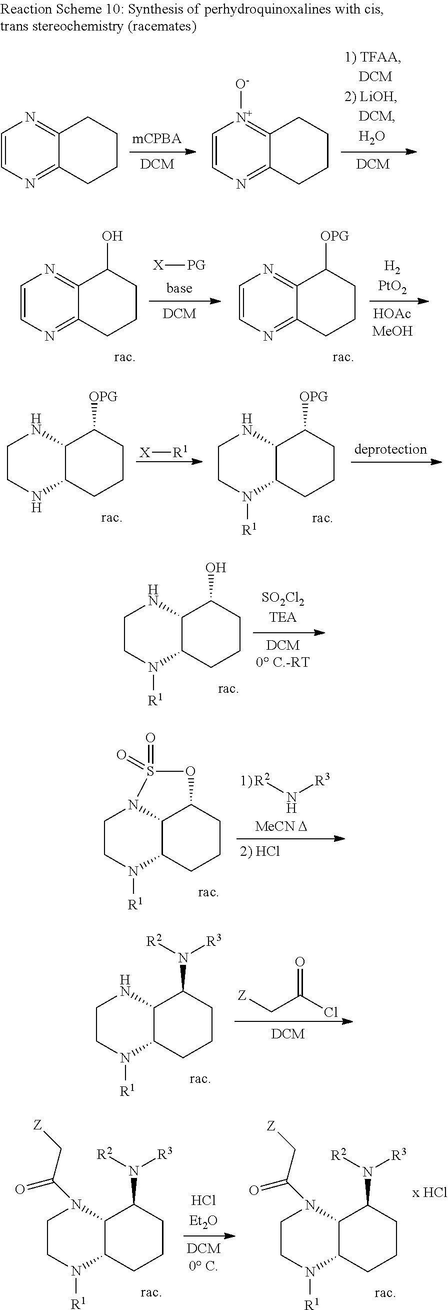

Optionally substituted perhydroquinoxalines with cis,trans stereochemistry can be obtained as shown in Reaction Scheme 10. 5,6,7,8-Tetrahydroquinoxaline can be oxidized with a peracid such as meta-chloroperbenzoic acid in a solvent like DCM to yield the corresponding N-oxides. Acylation with a reagent such as trifluoroacetic anhydride in a solvent like DCM followed by treatment with a base like lithium hydroxide in a mixture of water and DCM yields racemic 5,6,7,8-tetrahydroquinoxalin-5-ol. The alcohol function in benzylic position can be protected with a bulky protecting group PG by reaction with a reagent X-PG such as tert-butyldimethylsilyl trifluoromethanesulfonate in the presence of a base like 2,6-lutidine in a solvent such as DCM. A stereoselective reduction of the pyrazine ring can be achieved by hydrogenating the protected 5,6,7,8-tetrahydroquinoxalin-5-ol with 5 bar hydrogen in the presence of a catalyst like platinum dioxide in a solvent such as a mixture of acetic acid and methanol. The product with cis,cis configuration, O-protected (4aSR,5RS,8aSR)-5-hydroxy-decahydroquinoxaline, is obtained exclusively. Various substituents R¹ can be introduced regioselectively by reacting O-protected (4aSR,5RS,8aSR)-5-hydroxy-decahydroquinoxaline with reagents X—R¹ in an inert solvent like DCM or THF with or without a base such as triethylamine Subsequently the hydroxy group is deprotected. A tert-butyldimethylsilyl protecting group, for example, can be removed by reaction with a reagent such as ammonium fluoride in a solvent like methanol at elevated temperature. The α,β-aminoalcohol thus obtained is reacted with sulfuryl chloride in the presence of a base like triethylamine in an inert solvent such as DCM at reduced temperature to yield the corresponding 1,2,3-oxathiazolidine 2,2-dioxide. The residue —NR²R³ can be introduced by reacting optionally substituted 1,2,3-oxathiazolidine 2,2-dioxide with an amine HNR²R³ in a solvent like acetonitrile at elevated temperature followed by treatment with an acid such as aqueous hydrochloric acid. The reaction takes place under inversion of the stereogenic center. Therefore, a compound with cis,trans substitution, optionally substituted (4aRS,5SR,8aSR)-5-amino-octahydroquinoxaline, is obtained exclusively. Acylation in 4-position can be performed by reacting optionally substituted (4aRS,5 SR,8 aSR)-5-amino-octahydroquinoxaline with an acid chloride Z—CH₂COCl in a solvent like DCM with or without the presence of a base such as DIEA. The target compounds can be used as such or being converted to pharmaceutically acceptable salts such as a hydrochloride by reacting the free base with the corresponding acid, e.g. hydrogen chloride in diethyl ether in a suitable solvent like DCM.

R¹ can be a protecting group, e.g. a Boc, Cbz, benzyl, allyl, Alloc group, which is orthogonal to PG and can be cleaved once the residues —NR²R³ and —COCH₂Z have been introduced. Subsequent reaction with reagents X—R¹ as described above yields the target compounds.

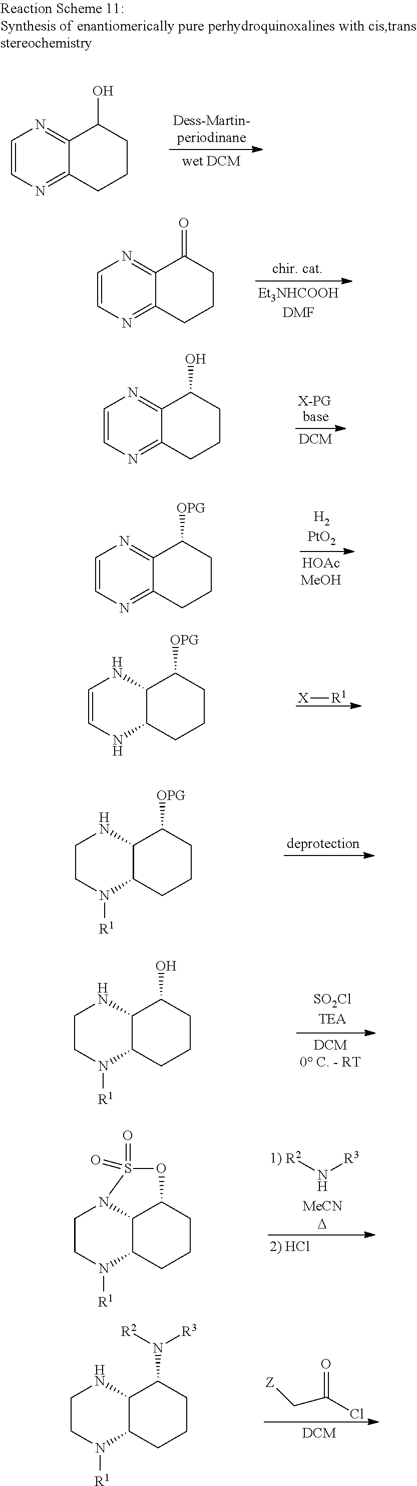

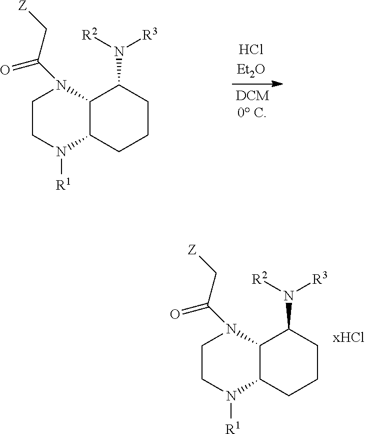

Enantiomerically pure, optionally substituted (4aR,5S,8aS)-octahydroquinoxalines with cis,trans stereochemistry can be obtained as shown in Reaction Scheme 11. Racemic 5,6,7,8-tetrahydroquinoxalin-5-ol can be oxidized to the corresponding ketone with a reagent such as Dess-Martin periodinane in a suitable solvent like wet DCM. Subsequently, the ketone is subjected to a asymmetric hydrogen transfer reaction with dichloro(p-cymene)ruthenium(II) dimer, (1R,2R)-N-p-tosyl-1,2-diphenylethylenediamine and triethylammonium formate in DMF to yield enantiomerically pure (R)-5,6,7,8-tetrahydroquinoxalin-5-ol. Alternatively, the reaction can be carried out using borane DMS complex or boran THF complex in the presence of (S)-Me-CBS-oxazoborolidine in a solvent like THF. All following steps are performed as described above for the racemate.

### EXAMPLES

The following describes the preparation of the detailed examples of the invention via reaction schemes 1 to 11 and their analysis.

Analytical LC-MS

Analytical conditions summary:

LC system: Agilent 1100; binary pump: Agilent G1312A; degasser; auto sampler; column heater.

Detector DAD: Agilent G1315D, 210 nm and 220-320 nm

MSD system: Agilent LC/MSD G6130B ESI (pos/neg) mass range: 100-800

Method A1:

Column Waters XBridge™ (C18, 50×2.1 mm, 3.5 μm); temperature: 35° C.; flow rate: 0.8 ml/min, gradient: t₀=2% A, t_{3.5 min}=98% A, t_{6 min}=98% A; post time: 2 minutes; eluent A: 0.1% formic acid in acetonitrile; eluent B: 0.1% formic acid in water; 220 and 220-320 nm

Method A2:

Column Waters XSelect™ (C18, 50×2.1 mm, 3.5 μm); temperature: 35° C.; flow rate: 0.8 ml/min, gradient: t₀=2% A, t_{3.5 min}=98% A, t_{6 min}=98% A; post time: 2 minutes; eluent A: 0.1% formic acid in acetonitrile; eluent B: 0.1% formic acid in water; 220 and 220-320 nm

Method A3:

Column Waters XSelect™ (C18, 150×4.6 mm, 3.5 μm); temperature: 35° C.; flow rate: 1 ml/min, gradient: t₀=5% A, t₁=5% A t_{10 min}=98% A, t_{15 min}=98% A; post time: 5 minutes; eluent A: 0.1% formic acid in acetonitrile; eluent B: 0.1% formic acid in water; 220-320 nm

Method B1:

Column Waters XBridge™ (C18, 50×2.1 mm, 3.5 μm); temperature: 25° C., flow rate: 0.8 ml/min, gradient: t₀=2% A, t_{3.5 min}=98% A, t_{6 min}=98% A; post time: 2 min, eluent A: 95% acetonitrile+5% 10 mM ammonium bicarbonate in water in acetonitrile, eluent B: 10 mM ammonium bicarbonate in water (pH=9.5); 220-320 nm

Method B2:

Column Waters XBridge™ (C18, 50×2.1 mm, 3.5 μm); temperature: 25° C., flow rate: 0.8 ml/min, gradient: t₀=2% A, t_{3.5 min}=98% A, t_{6 min}=98% A; post time: 2 min, eluent A: 95% acetonitrile+5% 10 mM ammonium bicarbonate in water in acetonitrile, eluent B: 10 mM ammonium bicarbonate in water (pH=9.5); 220 nm

Method B3:

Column Waters XBridge™ (C18, 50×2.1 mm, 3.5 μm); temperature: 25° C., flow rate: 0.8 ml/min, gradient: t₀=2% A, t_{3.5 min}=98% A, t_{6 min}=98% A; post time: 2 min, eluent A: 95% acetonitrile+5% 10 mM ammonium bicarbonate in water in acetonitrile, eluent B: 10 mM ammonium bicarbonate in water (pH=9.5); 210 nm

Method B4:

Column Waters XSelect™ column (C18, 50×2.1 mm, 3.5 μm); temperature: 25° C., flow rate: 0.8 ml/min, gradient: t₀=2% A, t_{3.5 min}=98% A, t_{6 min}=98% A; post time: 2 min, eluent A: 95% acetonitrile+5% 10 mM ammonium bicarbonate in water in acetonitrile, eluent B: 10 mM ammonium bicarbonate in water (pH=9.5); 220-320 nm

Method B5:

Column Waters XSelect™ column (C18, 50×2.1 mm, 3.5 μm); temperature: 25° C., flow rate: 0.8 ml/min, gradient: t₀=2% A, t_{3.5 min}=98% A, t_{6 min}=98% A; post time: 2 min, eluent A: 95% acetonitrile+5% 10 mM ammonium bicarbonate in water in acetonitrile, eluent B: 10 mM ammonium bicarbonate in water (pH=9.5); 220 nm

Structures of all diastereoisomers of the compounds of the invention such as of methyl 4-(2-(3,4-dichlorophenyl)acetyl)-5-(pyrrolidin-1-yl)octahydroquinoxaline-1(2H)-carboxylate hydrochloride were assigned using ¹H, COSY, NOESY, HMBC and HSQC NMR experiments.

Structures of all other examples of the present invention were confirmed with ¹H NMR experiments.

The compounds obtained according to the present invention are summarized in Tables 1 and 2 below.

TABLE 1 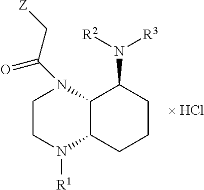
racemate

| | | | | | | MS | |
|---|---|---|---|---|---|---|---|
| | | | | | | MW | |
| | | | | HPLC | | (calc.) | |
| | | | | t_{R} | | free | [M + H⁺] |
| No. | R¹ | —NR²R³ | Z | (min) | method | base | (found) |
| 1 | 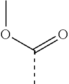 | 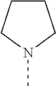 | 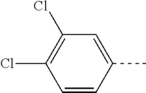 | 3.88 | B1 | 454.40 | 454 |
| 2 | 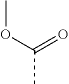 | 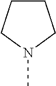 | 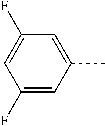 | 3.63 | B1 | 421.49 | 422 |
| 3 | 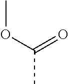 | 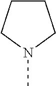 | 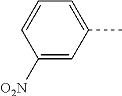 | 3.49 | B1 | 430.51 | 431 |
| 4 | 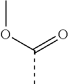 | 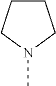 | 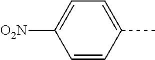 | 3.50 | B1 | 430.51 | 431 |
| 5 | 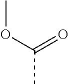 | 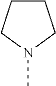 | 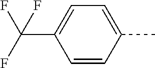 | 3.80 | B1 | 453.51 | 454 |
| 6 | 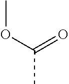 | 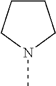 | 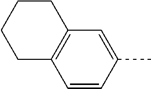 | 3.97 | B1 | 439.60 | 440 |
| 7 | 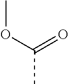 | 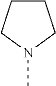 | 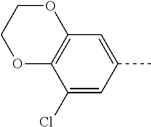 | 3.59 | B1 | 477.99 | 478 |
| 8 | 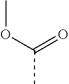 | 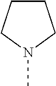 | 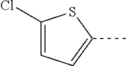 | 3.74 | B1 | 425.98 | 426 |
| 9 | 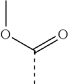 | 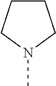 | 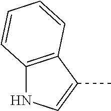 | 3.96 | B1 | 424.55 | 425 |
| 10 | 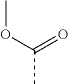 | 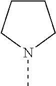 | 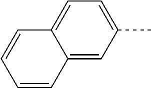 | 4.33 | B1 | 435.57 | 436 |
| 11¹⁾ | 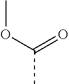 | 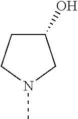 | 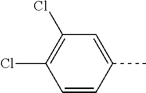 | 3.46 | B1 | 470.40 | 470 |
| 12 | 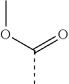 | 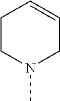 | 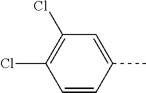 | 4.21 | B1 | 466.41 | 466 |
| 13 | 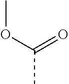 | 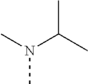 | 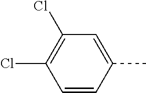 | 4.25 | B1 | 456.42 | 456 |
| 14 | 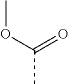 | 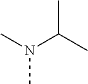 | 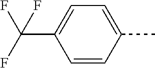 | 4.15 | B3 | 455.52 | 456 |
| 15 | 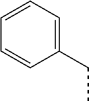 | 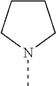 | 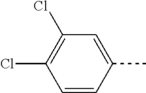 | 4.77 | B3 | 486.49 | 486 |
| 16³⁾ | 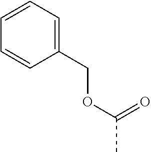 | 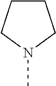 | 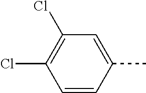 | 4.44 | B3 | 530.50 | 530 |
| 17 | 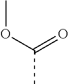 | 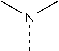 | 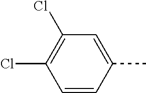 | 3.77 | B1 | 428.36 | 428 |
| 18 | 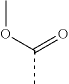 | 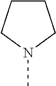 | 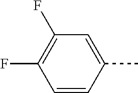 | 3.67 | B1 | 421.49 | 422 |
| 19 | 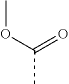 | 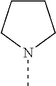 | 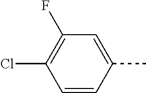 | 3.84 | B1 | 437.95 | 438 |
| 20 | 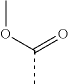 | 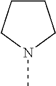 | 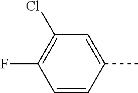 | 3.80 | B1 | 437.95 | 438 |
| 21 | 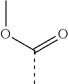 | 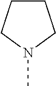 | 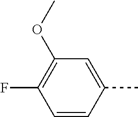 | 3.53 | B1 | 433.53 | 434 |
| 22 | 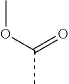 | 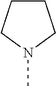 | 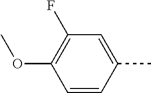 | 3.53 | B1 | 433.53 | 434 |
| 23 | 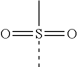 | 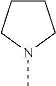 | 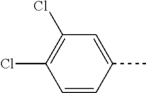 | 4.00 | B1 | 474.45 | 474 |
| 24³⁾ | 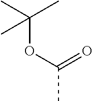 | 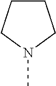 | 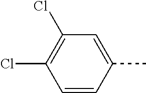 | 4.36 | B1 | 496.48 | 496 |
| 25 | 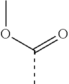 | 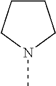 | 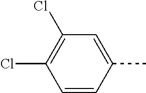 | 3.82 | B1 | 452.43 | 452 |
| 26 | 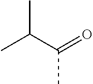 | 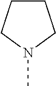 | 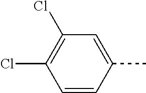 | 3.87 | B1 | 466.46 | 466 |
| 27 | 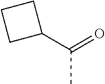 | 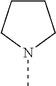 | 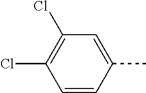 | 3.97 | B1 | 478.47 | 478 |
| 28 | 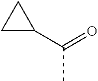 | 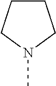 | 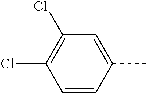 | 3.83 | B1 | 464.44 | 464 |
| 29 | 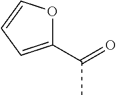 | 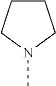 | 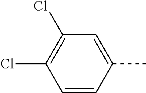 | 3.92 | B1 | 490.43 | 490 |
| 30 | 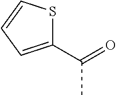 | 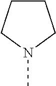 | 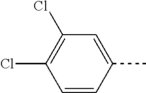 | 4.06 | B1 | 506.50 | 506 |
| 31 | 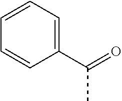 | 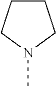 | 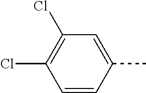 | 4.08 | B1 | 500.47 | 500 |
| 32 | 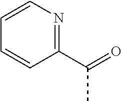 | 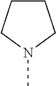 | 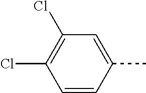 | 3.81 | B1 | 501.46 | 501 |
| 33 | 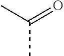 | 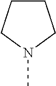 | 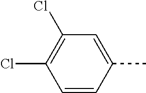 | 3.69 | B1 | 438.40 | 438 |
| 34 | 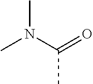 | 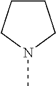 | 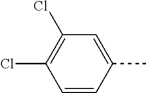 | 3.93 | B1 | 467.44 | 467 |
| 35 | 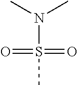 | 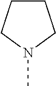 | 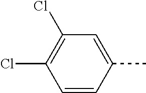 | 4.21 | B1 | 503.50 | 503 |
| 36 | 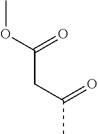 | 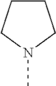 | 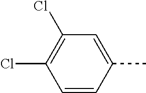 | 3.69 | B1 | 496.44 | 496 |
| 37 | 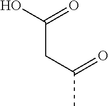 | 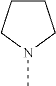 | 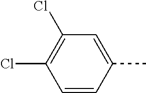 | 3.01 | B1 | 482.41 | 482 |
| 38 | 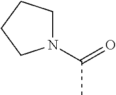 | 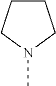 | 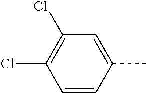 | 4.07 | B1 | 493.48 | 493 |
| 39³⁾ | 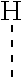 | 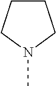 | 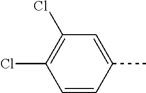 | 3.50 | B1 | 396.36 | 396 |
| 40 | 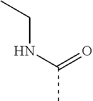 | 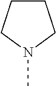 | 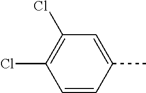 | 3.60 | B1 | 467.44 | 467 |
| 41 | 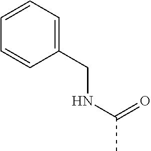 | 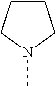 | 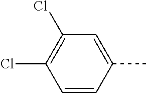 | 3.93 | B1 | 529.51 | 592 |
| 42 | 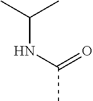 | 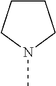 | 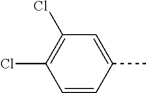 | 3.75 | B1 | 481.47 | 481 |
| 43 | 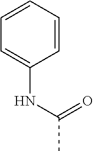 | 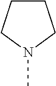 | 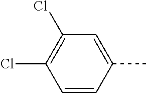 | 3.96 | B1 | 515.49 | 515 |
| 44²⁾ | 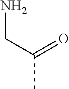 | 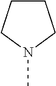 | 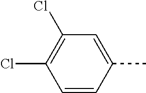 | 3.39 | B1 | 453.42 | 453 |
| 45 | 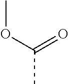 | 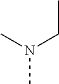 | 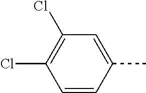 | 3.97 | B1 | 442.39 | 442 |
| 46 | 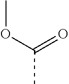 | 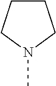 | 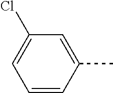 | 3.73 | B3 | 419.96 | 420 |
| 47 | 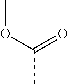 | 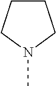 | 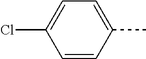 | 3.73 | B1 | 419.96 | 420 |
| 48²⁾ | 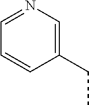 | 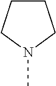 | 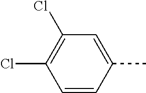 | 3.95 | B1 | 487.48 | 487 |
| 49²⁾ | 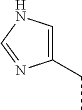 | 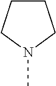 | 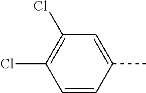 | 3.93 | B1 | 476.45 | 476 |
| 50 | 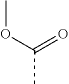 | 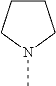 | 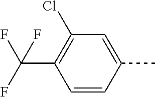 | 4.01 | B1 | 487.95 | 488 |
| 51 | 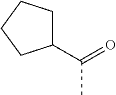 | 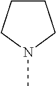 | 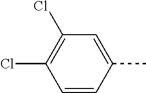 | 4.06 | B1 | 492.49 | 492 |
| 52 | 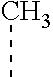 | 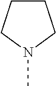 | 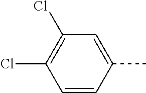 | 3.85 | B1 | 410.39 | 410 |
| 53¹⁾ | 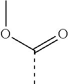 | 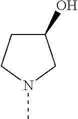 | 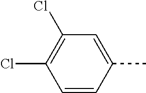 | 3.43 | B1 | 470.40 | 470 |
| 54 | 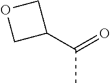 | 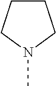 | 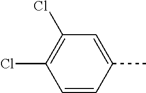 | 3.56 | B1 | 480.44 | 480 |
| 55¹⁾ | 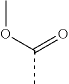 | 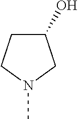 | 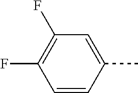 | 3.17 | B1 | 437.49 | 438 |
| 56¹⁾ | 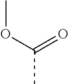 | 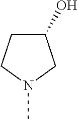 | 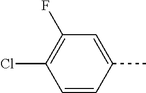 | 3.30 | B1 | 453.95 | 454 |
| 57³⁾ | 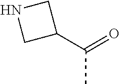 | 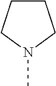 | 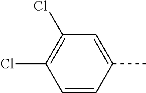 | 3.66 | B1 | 479.45 | 479 |
| 58 | 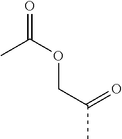 | 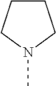 | 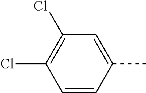 | 3.75 | B2 | 496.44 | 496 |
| 59 | 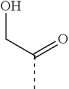 | 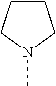 | 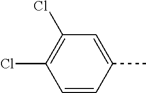 | 3.56 | B5 | 454.40 | 454 |
| 60³⁾ | 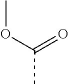 | 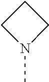 | 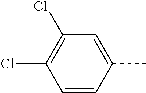 | 3.65 | B2 | 440.37 | 440 |
| 61 | 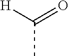 | 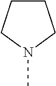 | 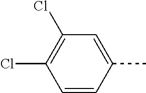 | 3.71 | B5 | 424.37 | 424 |
| 62 | 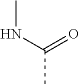 | 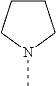 | 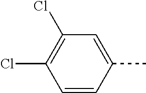 | 3.60 | B5 | 453.42 | 453 |
| 63³⁾ | 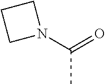 | 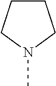 | 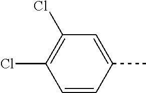 | 3.81 | B5 | 479.45 | 479 |
| 64³⁾ | 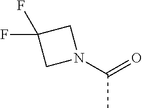 | 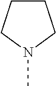 | 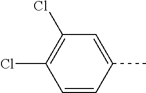 | 4.02 | B5 | 515.44 | 515 |
| 65 | 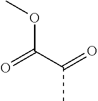 | 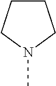 | 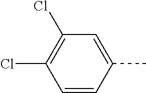 | 3.97 | B5 | 482.41 | 482 |
| 66 | 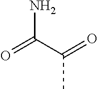 | 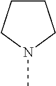 | 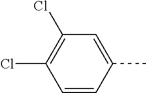 | 3.54 | B5 | 467.40 | 467 |
| 67 | 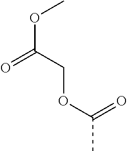 | 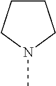 | 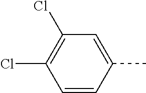 | 4.05 | B5 | 512.44 | 512 |
| 68 | 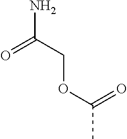 | 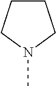 | 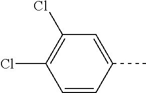 | 3.57 | B5 | 497.43 | 497 |
| 69 | 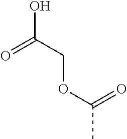 | 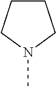 | 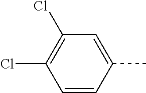 | 3.04 | B5 | 498.41 | 498 |
| 70²⁾ | 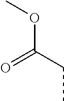 | 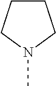 | 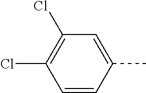 | 4.18 | B2 | 468.43 | 468 |
| 71³⁾ | 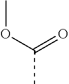 | 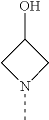 | 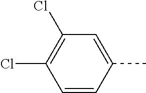 | 3.32 | B5 | 456.37 | 456 |
| 72⁴⁾ | 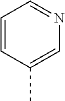 | 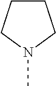 | 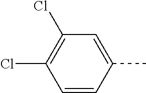 | 4.11 | B5 | 473.45 | 473 |
| 73²⁾ | 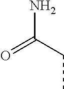 | 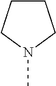 | 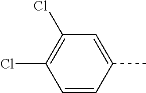 | 3.51 | B5 | 453.42 | 453 |
| 74²⁾ | 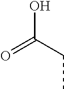 | 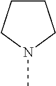 | 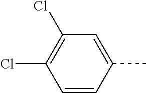 | 2.92 | B5 | 454.40 | 454 |
| 75 | 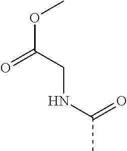 | 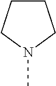 | 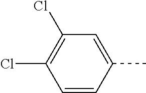 | 3.61 | B5 | 511.45 | 511 |
| 76 | 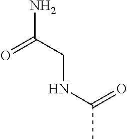 | 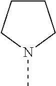 | 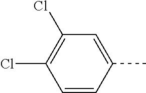 | 3.35 | B5 | 496.44 | 496 |
| 77 | 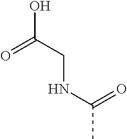 | 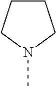 | 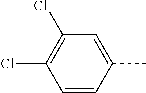 | 2.99 | B5 | 497.43 | 497 |
| 78 | 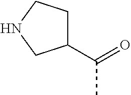 | 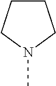 | 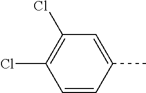 | 4.22 | B5 | 493.48 | 493 |
| 79 | 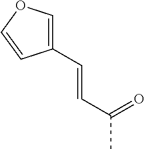 | 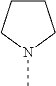 | 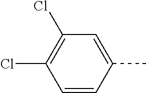 | 4.07 | B5 | 516.47 | 516 |
| 80 | 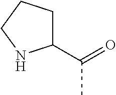 | 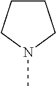 | 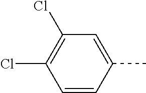 | 4.02 | B5 | 493.48 | 493 |
| 81 | 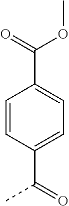 | 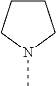 | 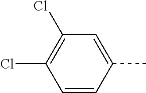 | 4.17 | B5 | 558.51 | 558 |
| 82 | 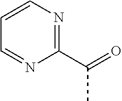 | 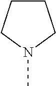 | 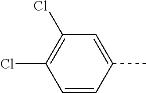 | 3.62 | B5 | 502.45 | 502 |
| 83 | 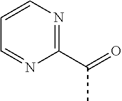 | 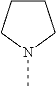 | 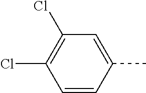 | 3.74 | B5 | 502.45 | 502 |
| 84⁵⁾ | 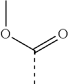 | 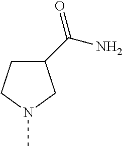 | 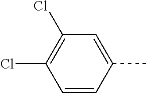 | 3.35 | B5 | 497.43 | 497 |
| 85 | 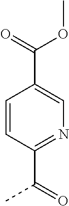 | 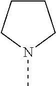 | 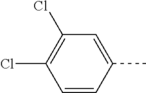 | 3.91 | B5 | 559.50 | 559 |
| 86 | 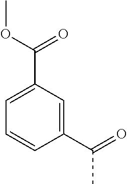 | 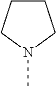 | 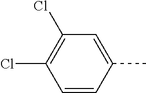 | 4.24 | B5 | 558.51 | 558 |
| 87 | 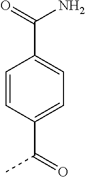 | 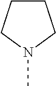 | 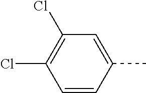 | 3.57 | B5 | 543.50 | 543 |
| 88³⁾ | 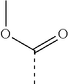 | 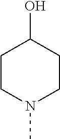 | 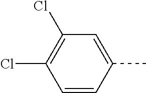 | 3.44 | B5 | 484.43 | 484 |

¹⁾mixture of two diastereoisomers
²⁾×2 HCl
³⁾free base
⁴⁾×3 HCl
⁵⁾mixture of four stereoisomers

TABLE 2

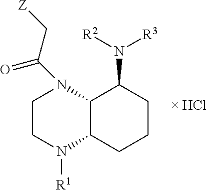

| | | | | | | MS | |
|---|---|---|---|---|---|---|---|
| | | | | | | MW | |
| | | | | HPLC | | (calc.) | |
| | | | | t_{R} | | free | [M + H⁺] |
| No. | R¹ | —NR²R³ | Z | (min) | method | base | (found) |
| 89 | 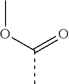 | 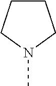 | 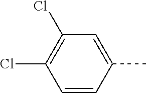 | 3.90 | B1 | 454.40 | 454 |
| 90 | 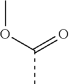 | 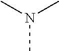 | 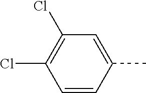 | 3.77 | B2 | 428.36 | 428 |
| 91 | 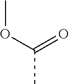 | 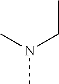 | 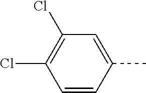 | 4.04 | B2 | 442.39 | 442 |
| 92 | 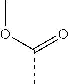 | 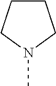 | 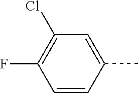 | 3.83 | B2 | 437.95 | 438 |
| 93 | 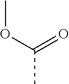 | 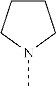 | 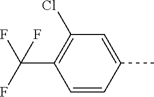 | 4.08 | B5 | 487.95 | 488 |
| 94 | 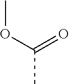 | 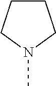 | 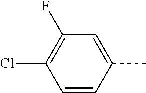 | 3.86 | B2 | 437.95 | 438 |
| 95 | 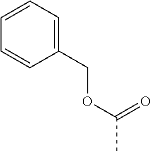 | 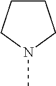 | 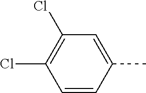 | 4.54 | B5 | 530.50 | 530 |
| 96 | 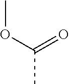 | 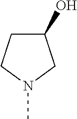 | 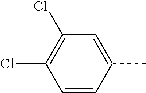 | 3.48 | B5 | 470.40 | 470 |
| 97 | 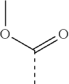 | 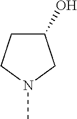 | 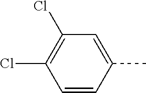 | 3.48 | B5 | 470.40 | 470 |
| 98 | 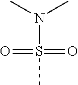 | 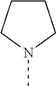 | 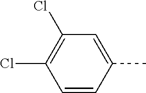 | 4.22 | B5 | 503.50 | 503 |
| 99 | 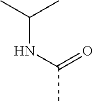 | 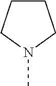 | 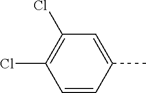 | 3.89 | B5 | 481.47 | 481 |
| 100 | 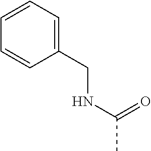 | 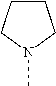 | 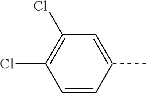 | 4.13 | B5 | 529.51 | 529 |
| 101 | 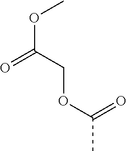 | 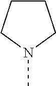 | 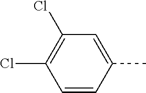 | 4.08 | B5 | 512.44 | 512 |
| 102 | 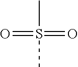 | 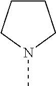 | 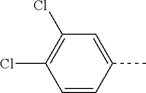 | 3.97 | B5 | 474.45 | 474 |
| 103¹⁾ | 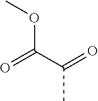 | 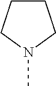 | 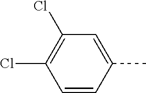 | 3.88 | B5 | 482.41 | 482 |
| 104¹⁾ | 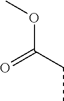 | 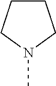 | 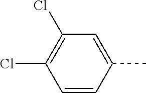 | 4.14 | B5 | 468.43 | 468 |
| 105 | 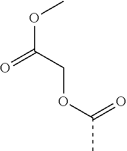 | 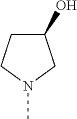 | 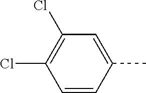 | 3.61 | B5 | 528.44 | 528 |
| 106 | 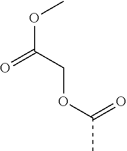 | 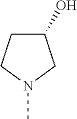 | 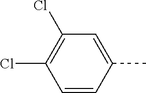 | 3.59 | B5 | 528.44 | 528 |
| 107 | 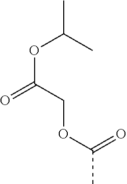 | 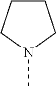 | 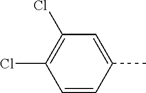 | 4.27 | B5 | 540.49 | 540 |
| 108 | 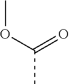 | 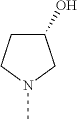 | 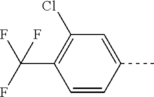 | 3.57 | B5 | 503.95 | 504 |
| 109 | 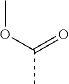 | 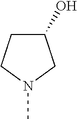 | 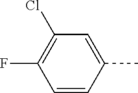 | 3.32 | B5 | 453.95 | 454 |
| 110¹⁾ | 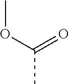 | 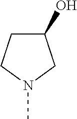 | 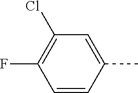 | 3.39 | B5 | 453.95 | 454 |
| 111¹⁾ | 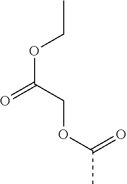 | 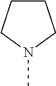 | 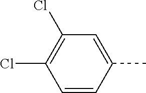 | 4.27 | B5 | 526.46 | 526 |
| 112 | 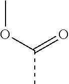 | 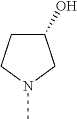 | 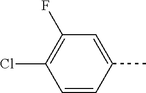 | 3.32 | B5 | 453.95 | 454 |
| 113 | 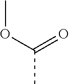 | 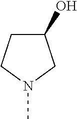 | 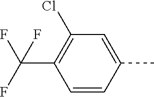 | 3.57 | B5 | 503.95 | 504 |
| 114¹⁾ | 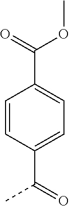 | 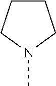 | 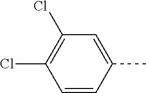 | 4.04 | B5 | 558.51 | 558 |
| 115¹⁾ | 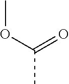 | 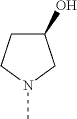 | 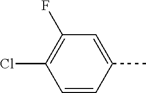 | 3.32 | B5 | 453.95 | 454 |
| 116¹⁾ | 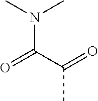 | 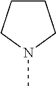 | 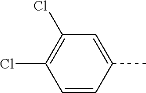 | 3.60 | B5 | 495.45 | 495 |
| 117¹⁾ | 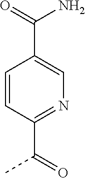 | 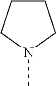 | 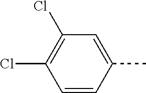 | 3.46 | B5 | 544.49 | 544 |
| 118¹⁾ | 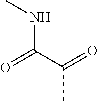 | 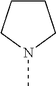 | 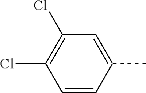 | 3.60 | B5 | 481.43 | 481 |
| 119¹⁾ | 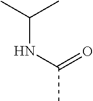 | 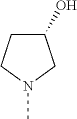 | 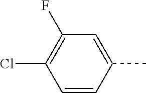 | 3.24 | B5 | 481.01 | 481 |
| 120¹⁾ | 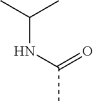 | 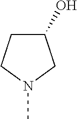 | 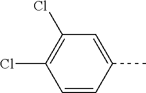 | 3.36 | B5 | 497.47 | 497 |
| 121¹⁾ | 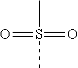 | 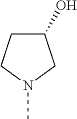 | 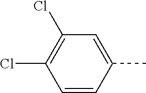 | 3.35 | B5 | 490.45 | 490 |
| 122¹⁾ | 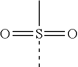 | 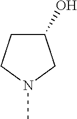 | 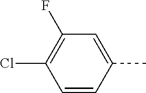 | 3.30 | B5 | 474.00 | 474 |
| 123¹⁾ | 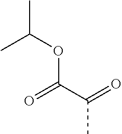 | 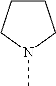 | 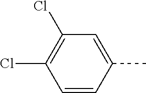 | 4.20 | B5 | 510.47 | 510 |
| 124¹⁾ | 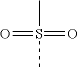 | 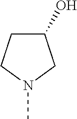 | 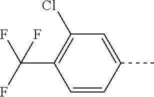 | 3.47 | B5 | 524.01 | 524 |
| 125¹⁾ | 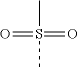 | 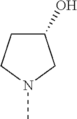 | 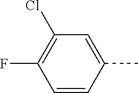 | 3.24 | B5 | 473.00 | 474 |
| 126¹⁾ | 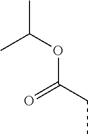 | 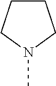 | 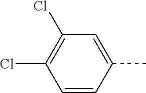 | 2.94 | A2 | 496.48 | 496 |
| 127¹⁾ | 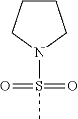 | 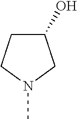 | 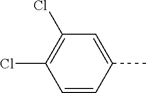 | 3.70 | B5 | 545.53 | 545 |
| 128¹⁾ | 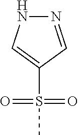 | 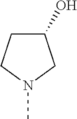 | 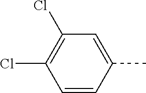 | 3.32 | B5 | 542.49 | 542 |
| 129¹⁾ | 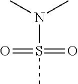 | 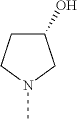 | 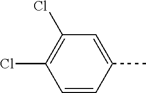 | 3.56 | B5 | 519.49 | 519 |
| 130¹⁾ | 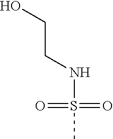 | 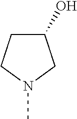 | 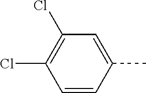 | 3.22 | B5 | 535.49 | 535 |
| 131¹⁾ | 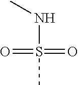 | 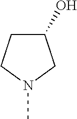 | 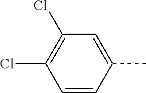 | 3.37 | B5 | 505.47 | 505 |
| 132¹⁾ | 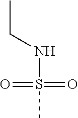 | 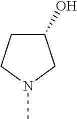 | 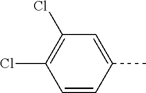 | 3.49 | B5 | 519.49 | 519 |
| 133¹⁾ | 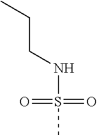 | 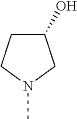 | 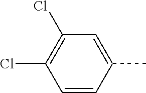 | 3.63 | B5 | 533.52 | 533 |
| 134¹⁾ | 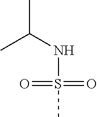 | 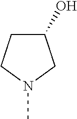 | 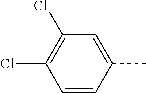 | 3.61 | B5 | 533.52 | 533 |
| 135¹⁾ | 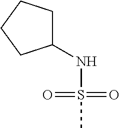 | 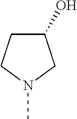 | 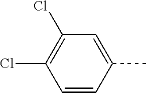 | 3.84 | B5 | 559.56 | 559 |
| 136¹⁾ | 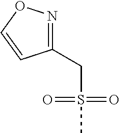 | 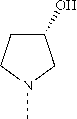 | 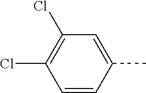 | 3.52 | B5 | 557.50 | 557 |
| 137¹⁾ | 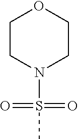 | 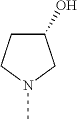 | 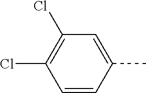 | 3.50 | B5 | 561.53 | 561 |
| 138 | 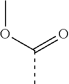 | 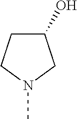 | 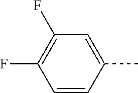 | 3.20 | B5 | 473.95 | 438 |
| 139 | 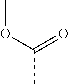 | 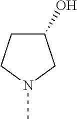 | 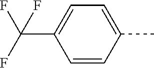 | 3.46 | B5 | 505.97 | 470 |
| 140 | 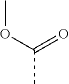 | 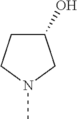 | 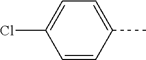 | 3.24 | B5 | 472.42 | 436 |
| 141 | 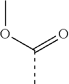 | 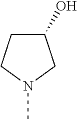 | 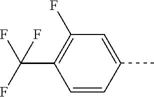 | 3.41 | B5 | 523.96 | 488 |
| 142 | 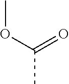 | 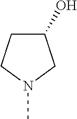 | 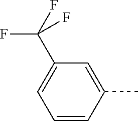 | 3.35 | B5 | 505.97 | 470 |
| 143 | 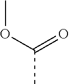 | 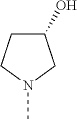 | 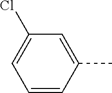 | 3.22 | B5 | 472.42 | 436 |
| 144¹⁾ | 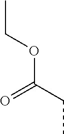 | 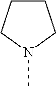 | 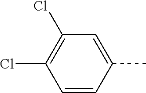 | 4.12 | B5 | 482.45 | 482 |
| 145¹⁾ | 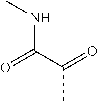 | 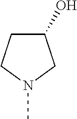 | 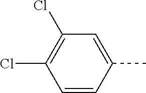 | 3.20 | B5 | 497.43 | 497 |
| 146¹⁾ | 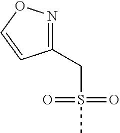 | 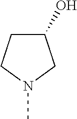 | 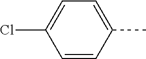 | 3.01 | B5 | 523.06 | 523 |
| 147¹⁾ | 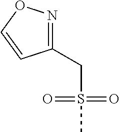 | 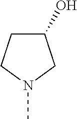 | 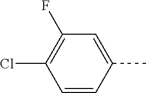 | 3.03 | B5 | 541.05 | 541 |
| 148¹⁾ | 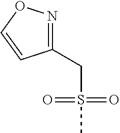 | 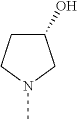 | 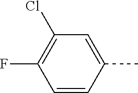 | 3.34 | B5 | 541.05 | 541 |
| 149¹⁾ | 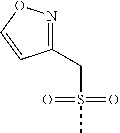 | 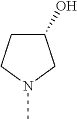 | 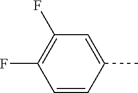 | 3.25 | B5 | 524.59 | 525 |
| 150¹⁾ | 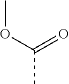 | 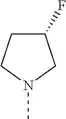 | 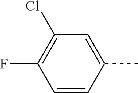 | 3.70 | B5 | 455.94 | 456 |

¹⁾free base

The following examples are provided to illustrate the invention and are not limiting the scope of the invention in any manner.

Synthesis of Reference Compounds B to D:

### (trans,trans)-2-Nitrocyclohexane-1,3-diol

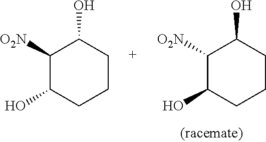 [SMILES: O=[N+]([O-])[C@H]1[C@H](O)CCC[C@@H]1O ; O=[N+]([O-])[C@H]1[C@H](O)CCC[C@@H]1O]

In a 2 l flask, a solution of glutaraldehyde 25% in water (182 ml) was mixed with methanol (600 ml). The reaction mixture was cooled to 0-5° C. and nitromethane (39.4 ml) was added. Sodium hydroxide 2 M (12 ml) was added dropwise. The cooling bath was removed and the reaction mixture was stirred at RT for 4 hours. The reaction mixture was “neutralized” (pH reached ˜5.35) with strong acidic cationic exchange resin (Amberlite IR120 H resin) and stirred for 20 minutes. The resin was filtered off and rinsed with MeOH. The filtrate was evaporated in vacuo. To the residue EtOH (100 ml) and toluene (250 ml) was added. The mixture was evaporated in vacuo. The solid residue was dissolved in hot EtOH (100 ml) and immediately toluene (250 ml) was added. The product precipitated and was filtered off After drying in vacuo 44.99 g product were obtained.

### (trans,trans)-N¹,N³-Dibenzyl-2-nitrocyclohexane-1,3-diamine

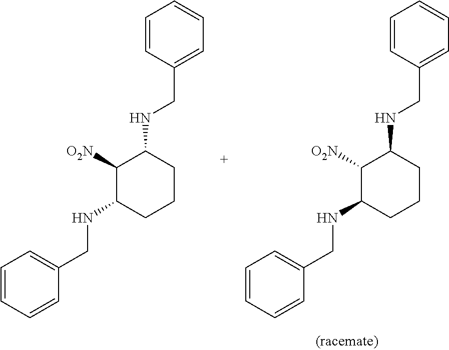 [SMILES: O=[N+]([O-])[C@@H]1[C@@H](NCc2ccccc2)CCC[C@H]1NCc1ccccc1 ; O=[N+]([O-])[C@@H]1[C@@H](NCc2ccccc2)CCC[C@H]1NCc1ccccc1]

In a 250 ml flask benzylamine (2.62 ml) was dissolved in water (60 ml) and (trans,trans)-2-nitrocyclohexane-1,3-diol (1.93 g) was added. The reaction mixture was stirred at RT overnight. An emulsion was obtained. The reaction mixture was stirred at RT over the weekend. Over the weekend the oil solidified on the surface of the flask. The solid was crushed with a spatula and was triturated for 2 hours. The precipitate was filtered off and recrystallized from hot MeOH (10 ml), affording 2.98 g product.

### (trans,trans)-N¹,N³-Dibenzylcyclohexane-1,2,3-triamine

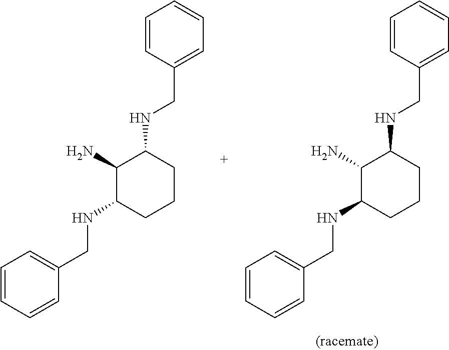 [SMILES: N[C@@H]1[C@@H](NCc2ccccc2)CCC[C@H]1NCc1ccccc1 ; N[C@@H]1[C@@H](NCc2ccccc2)CCC[C@H]1NCc1ccccc1]

A solution of (trans,trans)-N¹,N³-dibenzyl-2-nitrocyclohexane-1,3-diamine (2.98 g) in methanol (22 ml) was flushed with N₂ for at least 15 minutes. Raney nickel 50% slurry in water (4.51 ml) was added. The nitrogen was replaced by H₂ and the reaction mixture was stirred under a 1 bar H₂ atmosphere for 20 hours. The reaction mixture was filtered over diatomaceous earth and evaporated in vacuo, yielding 2.50 g product which was used as such for the next step.

### (trans,trans)-1-Benzyl-5-(benzylamino)hexahydroquinoxaline-2,3(1H,4H)-dione

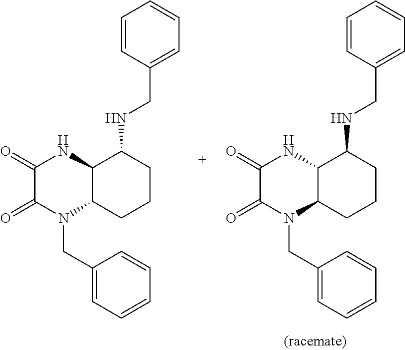 [SMILES: O=C1N[C@H]2[C@H](NCc3ccccc3)CCC[C@@H]2N(Cc2ccccc2)C1=O ; O=C1N[C@@H]2[C@@H](NCc3ccccc3)CCC[C@H]2N(Cc2ccccc2)C1=O]

A solution of (trans,trans)-N¹,N³-dibenzylcyclohexane-1,2,3-triamine (2.5 g) and dimethyl oxalate (0.954 g) in methanol (50 ml) was kept under reflux conditions for 24 h. The reaction mixture was evaporated in vacuo and coevaporated with EtOAc (3×). This afforded 2.9 g yellow brown solid residue. The residue was triturated with 60 ml boiling EtOAc. The mixture was cooled and partly evaporated in vacuo. The off white precipitate was filtered off and dried in vacuo to afford 2.05 g product.

### (trans,trans)-5-Amino-1-benzyloctahydroquinoxaline-2,3-dione

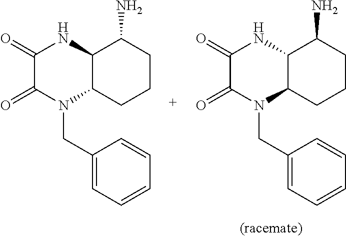 [SMILES: N[C@@H]1CCC[C@H]2[C@H]1NC(=O)C(=O)N2Cc1ccccc1 ; N[C@H]1CCC[C@@H]2[C@@H]1NC(=O)C(=O)N2Cc1ccccc1]

To a suspension of (trans,trans)-1-benzyl-5-(benzylamino)hexahydroquinoxaline-2,3(1H,4H)-dione (2.05 g) and ammonium formate (3.56 g) in methanol (40 ml) was carefully added palladium on carbon (0.210 g) in methanol (30 ml). The reaction mixture was stirred at reflux temperature for 2 hours. The reaction mixture was cooled to RT, filtered over diatomaceous earth and the residue washed thoroughly with MeOH. The filtrate was concentrated in vacuo and redissolved in CH₂Cl₂. The solution was washed with 0.1N NaOH (aq., 3×) and brine, dried over Na₂SO₄, filtered and concentrated in vacuo to yield 1.1 g of a white foam.

### (trans,trans)-5-Amino-1-benzyloctahydroquinoxaline-2,3-dione

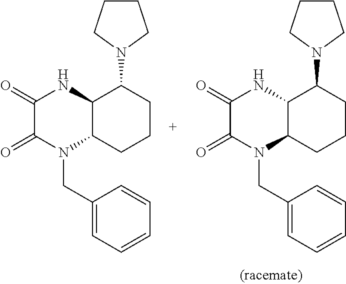 [SMILES: O=C1N[C@H]2[C@H](N3CCCC3)CCC[C@@H]2N(Cc2ccccc2)C1=O ; O=C1N[C@H]2[C@@H](CCC[C@@H]2N2CCCC2)N(Cc2ccccc2)C1=O]

To a mixture of 1.10 g (trans,trans)-5-amino-1-benzyloctahydroquinoxaline-2,3-dione and sodium hydrogen carbonate (2.299 g) in acetonitrile (100 ml), 1,4-diiodobutane (2.123 ml) was added. The mixture was kept under reflux condition for 40 h, filtered over diatomaceous earth and evaporated in vacuo. The residue was dissolved in CH₂Cl₂ and extracted three times with 150 ml 1N HCl (aq). The aqueous layers were combined and the pH was adjusted to 8 with 2N NaOH (aq.). The product was extracted with CH₂Cl₂ (3×), dried over Na₂SO₄, filtered and evaporated in vacuo. 1.18 g product were obtained, which was used as such in the next step.

### (trans,trans)-1-Benzyl-5-(pyrrolidin-1-yl)decahydroquinoxaline

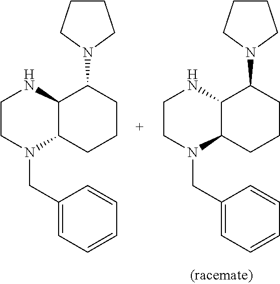 [SMILES: c1ccc(CN2CCN[C@H]3[C@H](N4CCCC4)CCC[C@@H]32)cc1 ; c1ccc(CN2CCN[C@H]3[C@H]2CCC[C@@H]3N2CCCC2)cc1]

At 0° C. under a nitrogen atmosphere aluminum chloride (0.820 g) was dissolved in dry tetrahydrofuran (50 ml). The clear colorless solution was stirred for 5 min at 0° C. and lithium aluminium hydride, 2.4 M in THF (7.77 ml) was added drop wise. The reaction mixture was stirred at RT for 20 min. The reaction mixture remained clear and colorless. Next, (trans,trans)-5-amino-1-benzyloctahydroquinoxaline-2,3-dione was dissolved in dry tetrahydrofuran (60 ml) and was added to the stirred mixture of Al(AlH₄)3 at 0° C. The reaction mixture was stirred at 0° C. for 60 min and during this time the reaction mixture turned slightly turbid. The reaction mixture was stirred at RT for 20 min, after which it was cooled with an ice/water bath and 2N NaOH (aq., 40 ml) was carefully added. The alkaline water layer was extracted with 100 ml CH₂Cl₂ (5×). The combined organic layer was dried over Na₂SO₄ and evaporated in vacuo. The crude product was purified by flash column chromatography (2% MeOH (NH₃) in CH₂Cl₂).

### 1-((trans,trans)-4-Benzyl-8-(pyrrolidin-1-yl)octahydroquinoxalin-1(2H)-yl)-2-(3,4-dichlorophenyl)ethanone

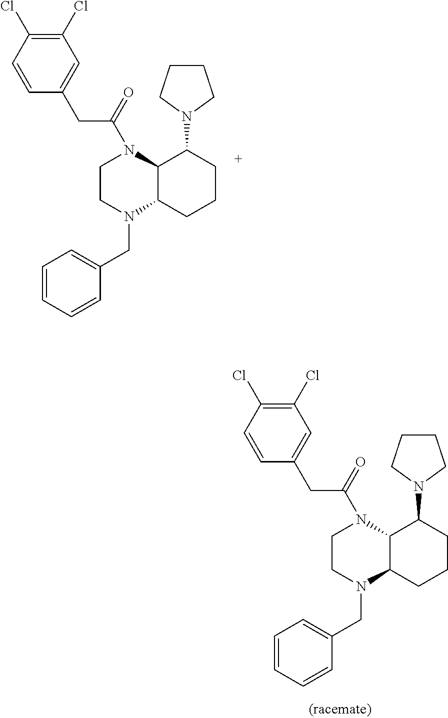 [SMILES: O=C(Cc1ccc(Cl)c(Cl)c1)N1CCN(Cc2ccccc2)[C@H]2CCC[C@@H](N3CCCC3)[C@@H]21 ; O=C(Cc1ccc(Cl)c(Cl)c1)N1CCN(Cc2ccccc2)[C@@H]2CCC[C@H](N3CCCC3)[C@H]21]

To a solution of (trans,trans)-1-benzyl-5-(pyrrolidin-1-yl)decahydroquinoxaline (325 mg) in dichloromethane (35 ml), 2-(3,4-dichlorophenyl)acetyl chloride (291 mg) was added. The reaction mixture was stirred at RT for 30 min. Next, 2N NaOH (aq., 35 ml) was added and the reaction mixture was stirred vigorously at RT for 2 hours. Phases were separated. The organic phase was extracted three times with 1N HCl (aq.). The pH of the acidic aqueous phase was adjusted to pH 8 with 2N NaOH (aq.) and subsequently extracted with CH₂Cl₂ (3×). The organic layers were combined and dried over Na₂SO₄, filtered and evaporated in vacuo. This afforded 497 mg product, which was used as such in the next step.

### 2-(3,4-Dichlorophenyl)-1-((trans,trans)-8-(pyrrolidin-1-yl)octahydroquinoxalin-1(2H)-yl)ethanone

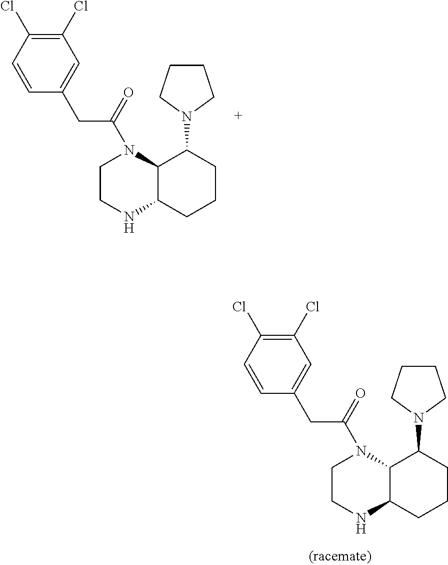 [SMILES: O=C(Cc1ccc(Cl)c(Cl)c1)N1CCN[C@H]2CCC[C@@H](N3CCCC3)[C@@H]21 ; O=C(Cc1ccc(Cl)c(Cl)c1)N1CCN[C@@H]2CCC[C@H](N3CCCC3)[C@H]21]

To a degassed solution of (trans,trans)-1-benzyl-5-(pyrrolidin-1-yl)decahydroquinoxaline (488 mg) in tetrahydrofuran (50 ml) and water (50 ml), hydrochloric acid 36% in H₂O (10 ml) was added followed by palladium, 10% on activated carbon (197 mg). The N₂ atmosphere was replaced by H₂ and the reaction mixture was stirred under a 1 bar H₂ atmosphere for 3 h. The reaction mixture was filtered over diatomaceous earth and the organic solvent was evaporated in vacuo. The acidic water layer was adjusted to pH 8 with 2N NaOH (aq.) and the water layer was extracted with CH₂Cl₂ (3×) The CH₂Cl₂ layers were combined and dried over Na₂SO₄, filtered and evaporated in vacuo. This afforded 386 mg product, which was used as such in the next step.

Reference Compound B

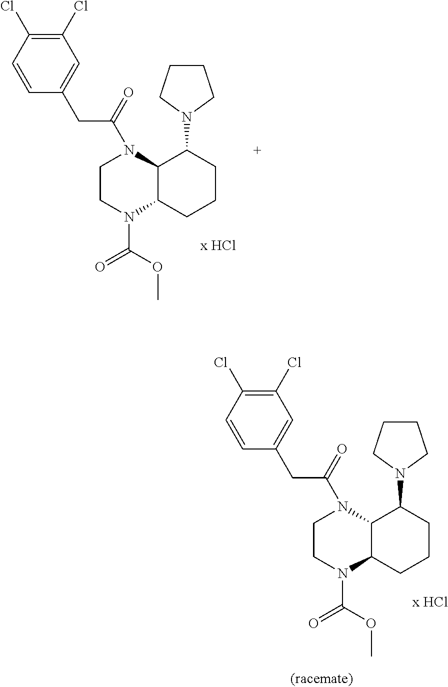 [SMILES: COC(=O)N1CCN(C(=O)Cc2ccc(Cl)c(Cl)c2)[C@H]2[C@H](N3CCCC3)CCC[C@@H]21 ; COC(=O)N1CCN(C(=O)Cc2ccc(Cl)c(Cl)c2)[C@H]2[C@H]1CCC[C@@H]2N1CCCC1]

To a solution of 2-(3,4-dichlorophenyl)-1-((trans,trans)-8-(pyrrolidin-1-yl)octahydro-quinoxalin-1(2H)-yl)ethanone (351 mg) in dichloromethane (20 ml), methyl chloroformate (0.086 ml) was added. The reaction mixture was stirred at RT overnight and evaporated in vacuo. This afforded 437 mg of an off white powder.

Reference Compounds C and D

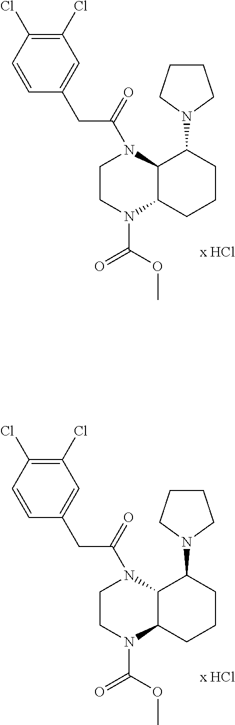 [SMILES: COC(=O)N1CCN(C(=O)Cc2ccc(Cl)c(Cl)c2)[C@H]2[C@H](N3CCCC3)CCC[C@@H]21 ; COC(=O)N1CCN(C(=O)Cc2ccc(Cl)c(Cl)c2)[C@H]2[C@H]1CCC[C@@H]2N1CCCC1]

Reference compound B (235 mg) was dissolved in CH₂Cl₂ and washed with sat. NaHCO₃ (aq.). The organic phase was collected using a phase separator and evaporated in vacuo. Coevaporating the residue with Et₂O afforded 205 mg of the free base as a white foam. The enantiomers were separated by chiral HPLC (Heptane:iPrOH 85:15 (0.4% diethylamine)). The fractions were evaporated in vacuo and coevaporated three times with CH₂Cl₂ followed by coevaporation (3×) with Et₂O. This afforded 71 mg colourless foamy oil of the enantiomer with the shorter retention time and 62 mg foamy oil of the enantiomer with the longer retention time. Both enantiomers were transformed back into the HCl salts (using HCl in Et₂O). This afforded 73 mg Reference compound C (obtained from the first eluting enantiomer on chiral-LC) and 70 mg Reference compound D (obtained from the second eluting enantiomer).

Synthesis of Reference Compound E:

### (R,S)-5-Bromo-5,6,7,8-tetrahydroquinoxaline

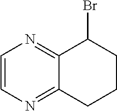 [SMILES: BrC1CCCc2nccnc21]

To a solution of 5,6,7,8-tetrahydroquinoxaline (15 g) and NBS (19.90 g) in carbon tetrachloride (500 ml), benzoyl peroxide (75%, remainder water; 0.271 g) was added. The reaction mixture was kept under reflux conditions overnight, filtered over diatomaceous earth and evaporated in vacuo. The residue was dissolved in CH₂Cl₂ and washed with sat. NaHCO₃ (aq.). The organic layer was dried over Na₂SO₄, filtered and evaporated in vacuo. Purification by gravity column chromatography (25% EtOAc in heptane) afforded 12.3 g product which was used as such for the next step.

### (R,S)-5-(Pyrrolidin-1-yl)-5,6,7,8-tetrahydroquinoxaline

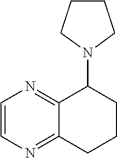 [SMILES: c1cnc2c(n1)CCCC2N1CCCC1]

To a solution of (R,S)-5-bromo-5,6,7,8-tetrahydroquinoxaline (12.3 g) and pyrrolidine (5.92 ml) in acetonitrile (120 ml), potassium carbonate (9.97 g) was added. The reaction mixture was stirred at RT overnight and evaporated in vacuo. The residue was redissolved in water/EtOAc. Phases were separated and the organic phase was washed with brine, dried over Na₂SO₄, filtered and evaporated in vacuo. The crude product was purified by flash column chromatography (1-4% (7N NH₃ in MeOH)/CH₂Cl₂) to afforded 9.3 g product.

### (cis,cis)-5-(Pyrrolidin-1-yl)decahydroquinoxaline

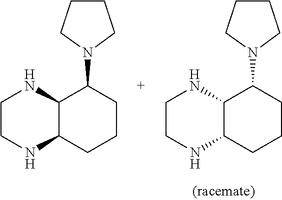 [SMILES: C1C[C@H]2NCCN[C@H]2[C@@H](N2CCCC2)C1 ; C1C[C@@H]2NCCN[C@@H]2[C@H](N2CCCC2)C1]

To a nitrogen flushed solution of (R,S)-5-(pyrrolidin-1-yl)-5,6,7,8-tetrahydroquinoxaline (2.5 g) in trifluoroacetic acid (90 ml), platinum (IV) oxide (100 mg) was added. The nitrogen was replaced by a hydrogen atmosphere (1 bar) and the reaction mixture was stirred at RT. The atmosphere was replaced with fresh hydrogen, several times, while the reaction was monitored by GCMS until completion was reached. The reaction mixture was evaporated to dryness and coevaporated subsequently with CH₂Cl₂, MeOH and CH₂Cl₂. The residue was stirred in 1N NaOH (aq.) for 1 hour and was then extracted with EtOAc (3×). The combined EtOAc layer was dried over Na₂SO₄, filtered and evaporated in vacuo. The residue was redissolved in MeCN, after which the white precipitate was filtered off and discarded. The filtrate was evaporated in vacuo. This afforded 2.55 g product, which was used as such in the next step.

### (cis,cis)-tert-Butyl 5-(pyrrolidin-1-yl)octahydroquinoxaline-1(2H)-carboxylate

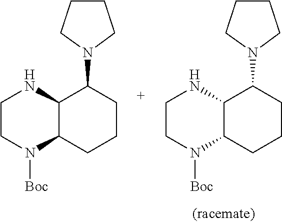 [SMILES: CC(C)(C)OC(=O)N1CCN[C@@H]2[C@H]1CCC[C@@H]2N1CCCC1 ; CC(C)(C)OC(=O)N1CCN[C@@H]2[C@H](N3CCCC3)CCC[C@@H]21]

To a solution of (cis,cis)-5-(pyrrolidin-1-yl)decahydroquinoxaline (2.55 g) in dichloromethane (250 ml), di-tert-butyl dicarbonate (2.92 g) was added. The reaction mixture was stirred at RT for 20 hours and concentrated in vacuo. The crude product was purified by flash column chromatography (CH₂Cl₂:MeOH(NH₃) 98:2, ninhydrine) this afforded 1.15 g with 84% purity and 460 mg product with 90% purity (GCMS). The 1.15 g batch was further purified by gravity column chromatography, giving 640 mg product with 96% purity (GCMS).

### (cis,cis)-tert-Butyl 4-(2-(3,4-dichlorophenyl)acetyl)-5-(pyrrolidin-1-yl)octahydro-quinoxaline-1(2H)-carboxylate

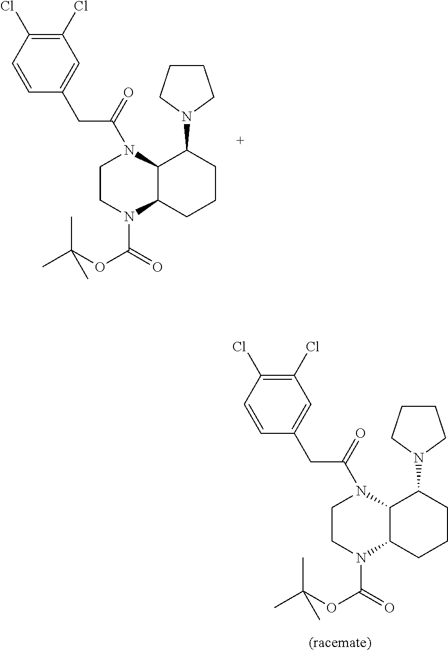 [SMILES: CC(C)(C)OC(=O)N1CCN(C(=O)Cc2ccc(Cl)c(Cl)c2)[C@@H]2[C@H]1CCC[C@@H]2N1CCCC1 ; CC(C)(C)OC(=O)N1CCN(C(=O)Cc2ccc(Cl)c(Cl)c2)[C@@H]2[C@H](N3CCCC3)CCC[C@@H]21]

To a solution of (cis,cis)-tert-butyl 5-(pyrrolidin-1-yl)octahydroquinoxaline-1(2H)-carboxylate (640 mg) and DIEA (0.531 ml) in dichloromethane (60 ml), 2-(3,4-dichlorophenyl)acetyl chloride (555 mg) was added. The reaction mixture was stirred at RT overnight. The reaction mixture was washed with sat. NaHCO₃ (aq.), and water, dried over Na₂SO₄, filtered and evaporated in vacuo. The crude product was purified by flash column chromatography (2% MeOH (NH₃) in CH₂Cl₂). This afforded 850 mg of product. LCMS analysis showed the product to consist of a mixture of diasteroisomers. The batch was further purified by preparative LCMS, yielding 475 mg of product.

### (cis,cis)-tert-Butyl 4-(2-(3,4-dichlorophenyl)acetyl)-5-(pyrrolidin-1-yl)octahydro-quinoxaline-1(2H)-carboxylate

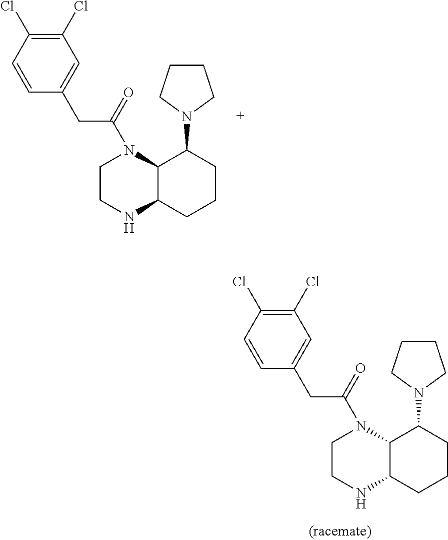 [SMILES: O=C(Cc1ccc(Cl)c(Cl)c1)N1CCN[C@@H]2CCC[C@H](N3CCCC3)[C@@H]21 ; O=C(Cc1ccc(Cl)c(Cl)c1)N1CCN[C@H]2CCC[C@@H](N3CCCC3)[C@H]21]

To a solution of (cis,cis)-tert-butyl 4-(2-(3,4-dichlorophenyl)acetyl)-5-(pyrrolidin-1-yl)octahydroquinoxaline-1(2H)-carboxylate (475 mg) in dichloromethane (2.25 ml), trifluoroacetic acid (2.34 ml) was added. The reaction mixture was stirred at RT for 30 min. The reaction mixture was concentrated in vacuo, redissolved in CH₂Cl₂ and washed with sat. NaHCO₃ (aq.). The organic layer was dried over Na₂SO₄, filtered and evaporated in vacuo to afford 364 mg product

Reference Compound E

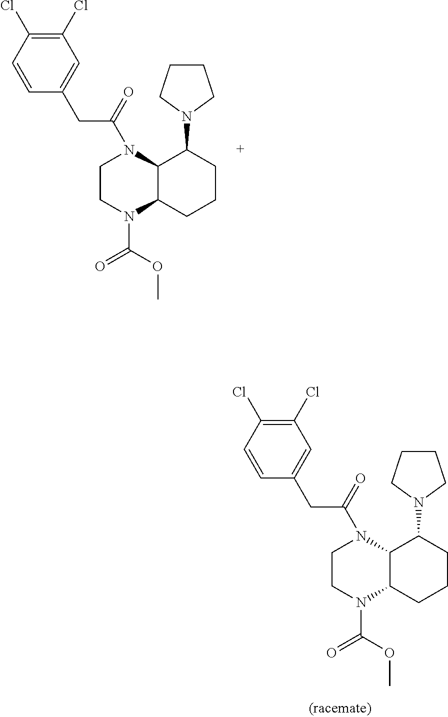 [SMILES: COC(=O)N1CCN(C(=O)Cc2ccc(Cl)c(Cl)c2)[C@@H]2[C@H]1CCC[C@@H]2N1CCCC1 ; COC(=O)N1CCN(C(=O)Cc2ccc(Cl)c(Cl)c2)[C@@H]2[C@H](N3CCCC3)CCC[C@@H]21]

To a solution of (cis,cis)-2-(3,4-dichlorophenyl)-1-(8-(pyrrolidin-1-yl)octahydroquinoxalin-1(2H)-yl)ethanone (364 mg) in dichloromethane (20 ml), methyl chloroformate (0.089 ml) was added. The reaction mixture was stirred at RT overnight and washed with sat. NaHCO₃ (aq.) solution and water. The organic layer was dried over Na₂SO₄, filtered and evaporated in vacuo. This afforded 360 mg product. Of this batch 23 mg was dissolved in CH₂Cl₂ (1 ml) and 1N HCl in Et₂O (2 ml) was added to convert the material to its HCl salt. The mixture was concentrated in vacuo and coevaporated twice with Et₂O. The residue was dried under reduced pressure at 40° C.

### Synthesis of Examples 1 and 89 and Reference compounds A, F and G

### 5-Nitroquinoxaline

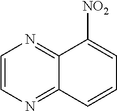 [SMILES: O=[N+]([O-])c1cccc2nccnc12]

A solution of 3-nitrobenzene-1,2-diamine (25 g) and glyoxal solution (40 wt % in water, 56.0 ml) in ethanol (96%, 400 ml) was kept under reflux conditions for 2 hours. The reaction mixture was concentrated in vacuo and water was added. The mixture was extracted with CH₂Cl₂ (3×). The combined organic layer was washed with brine, dried over Na₂SO₄, filtered and evaporated in vacuo. The crude product was purified by gravity column chromatography (EtOAc:heptane, 2:3) to yield 26.22 g product.

### Quinoxalin-5-amine

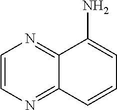 [SMILES: Nc1cccc2nccnc12]

A solution of 5-nitroquinoxaline (1.00 g) in ethanol (60 ml) was degassed with N₂ and palladium (10% on activated carbon, 0.061 g) was added. The N₂-atmosphere was replaced by H₂ and the reaction mixture was stirred under 1 bar H₂ atmosphere at RT overnight. The reaction mixture was filtered over diatomaceous earth and evaporated in vacuo. This afforded 845 mg crude product which was used us such for the next step

### 5-(Pyrrolidin-1-yl)quinoxaline

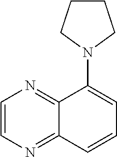 [SMILES: c1cc(N2CCCC2)c2nccnc2c1]

To a solution of quinoxalin-5-amine (11.7 g) in dry acetonitrile (1170 ml), sodium hydrogen carbonate (46.0 g) and 1,4-diiodobutane (42.5 ml) was added. The reaction mixture was kept under reflux conditions for 40 h. The reaction mixture was filtered over diatomaceous earth and concentrated in vacuo. The residue was dissolved in CH₂Cl₂ and extracted twice with a 1N HCl (aq.) solution. The pH of the aqueous layer was adjusted to pH 8-10 with 5N NaOH (aq.) and extracted with CH₂Cl₂ (3×). The combined organic layer was dried over Na₂SO₄, filtered and evaporated in vacuo. The residues was dissolved in EtOH and the dark impurities removed by filtration. The filtrate was evaporated in vacuo and purified by flash column chromatography (0% to 2% MeOH in CH₂Cl₂) yielding 4.16 g product which was used as such in the next step.

### 5-(Pyrrolidin-1-yl)-1,2,3,4-tetrahydroquinoxaline

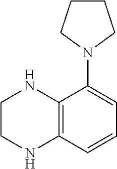 [SMILES: c1cc2c(c(N3CCCC3)c1)NCCN2]

Raney nickel (50% slurry in water, excess) was activated by washings with EtOH and added to a nitrogen flushed solution of 5-(pyrrolidin-1-yl)quinoxaline (4.16 g) and potassium hydroxide (0.276 g) in ethanol (75 ml). The nitrogen atmosphere was replaced by H₂ and the mixture was stirred at RT under a 1 bar H₂ atmosphere (balloon) for 21 hours. The reaction mixture was degassed with N₂ and filtered over diatomaceous earth. The filtrate was evaporated in vacuo and redissolved in Et₂O. Salts were removed by filtration and the filtrate was evaporated in vacuo. This afforded 4.1 g product.

### Methyl 5-(pyrrolidin-1-yl)-3,4-dihydroquinoxaline-1(2H)-carboxylate

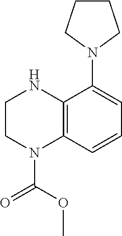 [SMILES: COC(=O)N1CCNc2c(N3CCCC3)cccc21]

A solution of methyl chloroformate (3.17 ml) in dichloromethane (15 ml) was added dropwise to an ice/water cooled solution of 5-(pyrrolidin-1-yl)-1,2,3,4-tetrahydroquinoxaline (4.16 g) and triethyl amine (3.70 ml) in dichloromethane (285 ml). The reaction mixture was stirred at RT for three days. Methyl chloroformate (0.793 ml) in dichloromethane (5 ml) was added to the reaction mixture and stirring was continued for 4 h. The reaction mixture was washed with sat. Na₂CO₃ (aq.) and brine. The CH₂Cl₂ layer was dried over Na₂SO₄, filtered and evaporated in vacuo. The crude product was purified by flash column chromatography (15% EtOAc in heptane) to yield 4.55 g product.

### (cis,trans)-Methyl 5-(pyrrolidin-1-yl)octahydroquinoxaline-1(2H)-carboxylate

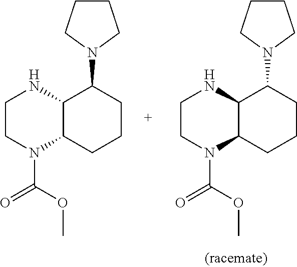 [SMILES: COC(=O)N1CCN[C@@H]2[C@@H](N3CCCC3)CCC[C@@H]21 ; COC(=O)N1CCN[C@H]2[C@H](N3CCCC3)CCC[C@H]21]

Platinum (IV) oxide (0.261 g) was added to a solution of methyl 5-(pyrrolidin-1-yl)-3,4-dihydroquinoxaline-1(2H)-carboxylate (1 g) in trifluoroacetic acid (20 ml) under nitrogen. The mixture was stirred under 1 bar H₂ atmosphere at RT for 4 h. The mixture was diluted with CH₂Cl₂ and concentrated. The residue was taken up in CH₂Cl₂, filtered, washed with 1M NaOH (aq.), dried over Na₂SO₄ and concentrated to afford 800 mg crude product. GCMS-analysis showed the presence of 1% starting material, 51% methyl (4aSR,8aRS)-octahydroquinoxaline-1(2H)-carboxylate and 2 peaks with the mass of methyl 5-(pyrrolidin-1-yl)octahydroquinoxaline-1(2H)-carboxylate (10% and 30%). Purification by flash chromatography (eluent 2-8-20% MeOH/CH₂Cl₂) afforded first 300 mg of pure methyl (4aSR,8aRS)-octahydroquinoxaline-1(2H)-carboxylate then 85 mg as a mixture of methyl (4aSR,8aRS)-octahydroquinoxaline-1(2H)-carboxylate and 2 isomers of methyl 5-(pyrrolidin-1-yl)octahydroquinoxaline-1(2H)-carboxylate (major one with shorter retention time on GCMS =(trans, c is)-methyl 5-(pyrrolidin-1-yl)octahydroquinoxaline-1(2H)-carboxylate) and 105 mg as only one isomer of methyl 5-(pyrrolidin-1-yl)octahydroquinoxaline-1(2H)-carboxylate (longer retention time). The 85 mg batch of methyl 5-(pyrrolidin-1-yl)octahydroquinoxaline-1(2H)-carboxylate was used for the next step.

### Example 1

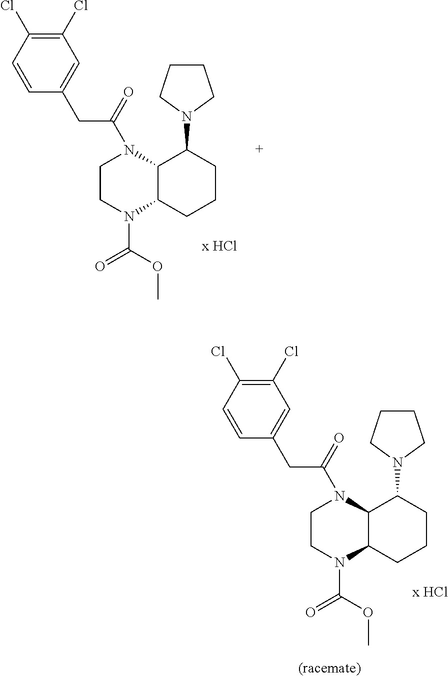 [SMILES: COC(=O)N1CCN(C(=O)Cc2ccc(Cl)c(Cl)c2)[C@@H]2[C@@H](N3CCCC3)CCC[C@@H]21 ; COC(=O)N1CCN(C(=O)Cc2ccc(Cl)c(Cl)c2)[C@H]2[C@H](N3CCCC3)CCC[C@H]21]

A solution of 2-(3,4-dichlorophenyl)acetyl chloride (107 mg) in dichloromethane (1 ml) was added dropwise to a solution of (cis,trans)-methyl 5-(pyrrolidin-1-yl)octahydroquinoxaline-1(2H)-carboxylate (85 mg) and triethyl amine (0.071 ml) in dichloromethane (3 ml). The mixture was stirred at RT for three days. The mixture was hydrolyzed with water, diluted with CH₂Cl₂, washed with sat. NaHCO₃ (aq.), dried over Na₂SO₄ and concentrated. Purification by flash column chromatography (1-2% (7N NH₃ in MeOH)/CH₂Cl₂) gave 82 mg of crude product. The product was purified a second time by flash chromatography (eluent (1% 7N NH₃ in MeOH)/CH₂Cl₂) gave 7 mg of product, which was converted to its HCl salt. 8 mg of Example 1 were thus obtained.

### Methyl 5-(pyrrolidin-1-yl)octahydroquinoxaline-1(2H)-carboxylate

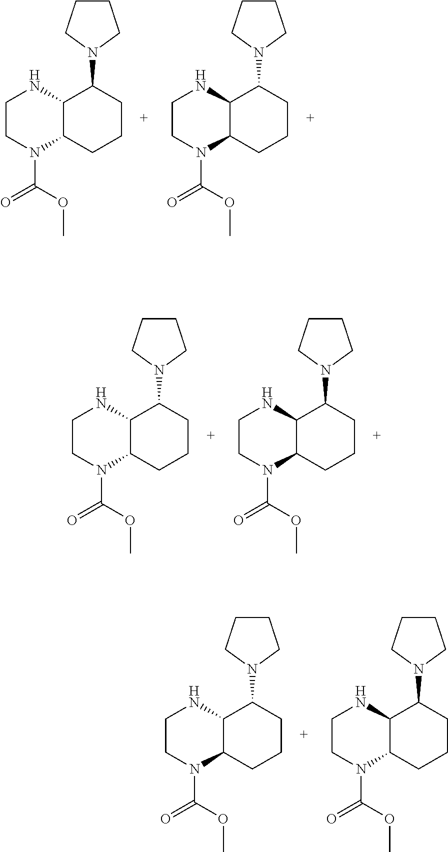 [SMILES: COC(=O)N1CCN[C@@H]2[C@@H](N3CCCC3)CCC[C@@H]21 ; COC(=O)N1CCN[C@H]2[C@H](N3CCCC3)CCC[C@H]21 ; COC(=O)N1CCN[C@@H]2[C@H](N3CCCC3)CCC[C@@H]21 ; COC(=O)N1CCN[C@@H]2[C@H]1CCC[C@@H]2N1CCCC1 ; COC(=O)N1CCN[C@@H]2[C@H](N3CCCC3)CCC[C@H]21 ; COC(=O)N1CCN[C@H]2[C@@H](N3CCCC3)CCC[C@@H]21]

Platinum (IV) oxide (0.521 g) was added to a solution of methyl 5-(pyrrolidin-1-yl)-3,4-dihydroquinoxaline-1(2H)-carboxylate (2.0 g) in degassed trifluoroacetic acid (50 ml) under nitrogen and the mixture was stirred under 1 bar H₂ atmosphere at RT for 3 h. The mixture was diluted with CH₂Cl₂, filtered and concentrated. The residue was taken up in CH₂Cl₂, washed with 1M NaOH (aq.), dried over Na₂SO₄, filtered and concentrated to afford 2.1 g crude product. GCMS-analysis showed the presence 65% (4aSR,8aRS)-octahydroquinoxaline-1(2H)-carboxylate, 2 peaks with the mass of methyl 5-(pyrrolidin-1-yl)octahydroquinoxaline-1(2H)-carboxylate (2% and 17%). Purification by flash chromatography (eluent 5-50% MeOH/CH₂Cl₂) afforded:

(c is ,trans)-methyl 5-(pyrrolidin-1-yl)octahydroquinoxaline-1(2H)-carboxylate (72 mg) (isomer with shortest retention time):

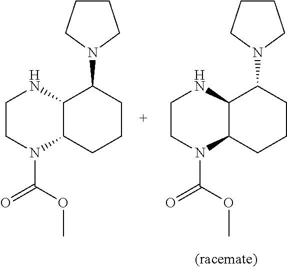 [SMILES: COC(=O)N1CCN[C@@H]2[C@@H](N3CCCC3)CCC[C@@H]21 ; COC(=O)N1CCN[C@H]2[C@H](N3CCCC3)CCC[C@H]21]

(cis,trans)-methyl 5-(pyrrolidin-1-yl)octahydroquinoxaline-1(2H)-carboxylate and methyl (4aSR,8aRS)-octahydroquinoxaline-1(2H)-carboxylate (54 mg):

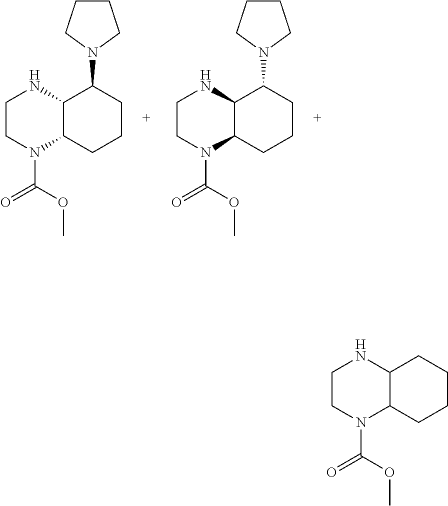 [SMILES: COC(=O)N1CCN[C@@H]2[C@@H](N3CCCC3)CCC[C@@H]21 ; COC(=O)N1CCN[C@H]2[C@H](N3CCCC3)CCC[C@H]21 ; COC(=O)N1CCNC2CCCCC21]

(cis,trans)-methyl 5-(pyrrolidin-1-yl)octahydroquinoxaline-1(2H)-carboxylate, methyl (4aSR,8aRS)-octahydroquinoxaline-1(2H)-carboxylate and (cis, c is)-methyl 5-(pyrrolidin-1-yl)octahydroquinoxaline-1 (2H)-carboxylate (125 mg):

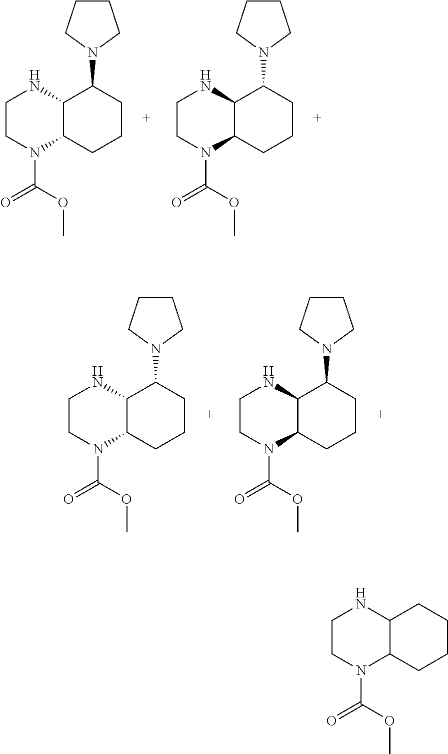 [SMILES: COC(=O)N1CCN[C@@H]2[C@@H](N3CCCC3)CCC[C@@H]21 ; COC(=O)N1CCN[C@H]2[C@H](N3CCCC3)CCC[C@H]21 ; COC(=O)N1CCN[C@@H]2[C@H](N3CCCC3)CCC[C@@H]21 ; COC(=O)N1CCN[C@@H]2[C@H]1CCC[C@@H]2N1CCCC1 ; COC(=O)N1CCNC2CCCCC21]

(trans,cis)-methyl 5-(pyrrolidin-1-yl)octahydroquinoxaline-1(2H)-carboxylate (270 mg) (isomer with longest retention time):

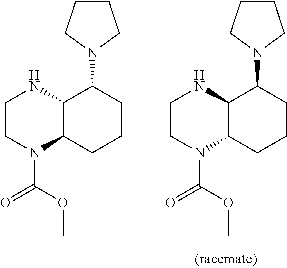 [SMILES: COC(=O)N1CCN[C@@H]2[C@H](N3CCCC3)CCC[C@H]21 ; COC(=O)N1CCN[C@H]2[C@@H](N3CCCC3)CCC[C@@H]21]

(trans,cis)-methyl 5-(pyrrolidin-1-yl)octahydroquinoxaline-1(2H)-carboxylate and methyl (4aSR,8aRS)-octahydroquinoxaline-1(2H)-carboxylate (50 mg):

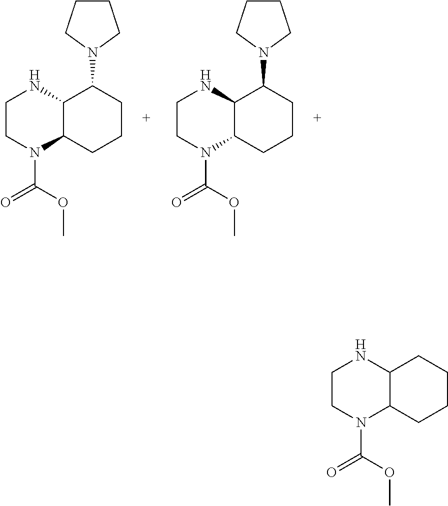 [SMILES: COC(=O)N1CCN[C@@H]2[C@H](N3CCCC3)CCC[C@H]21 ; COC(=O)N1CCN[C@H]2[C@@H](N3CCCC3)CCC[C@@H]21 ; COC(=O)N1CCNC2CCCCC21]

### Example 89 and Reference Compound A

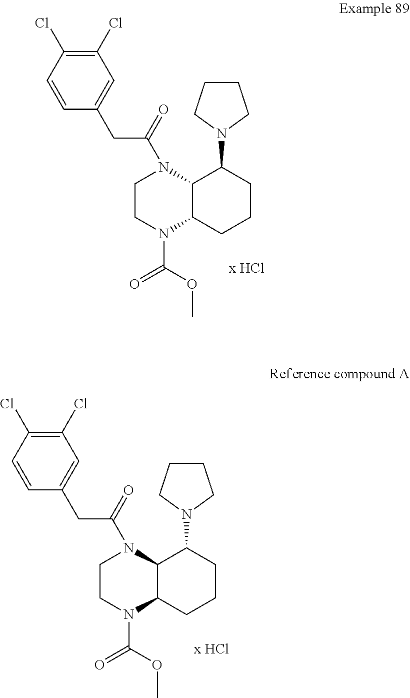 [SMILES: COC(=O)N1CCN(C(=O)Cc2ccc(Cl)c(Cl)c2)[C@@H]2[C@@H](N3CCCC3)CCC[C@@H]21 ; COC(=O)N1CCN(C(=O)Cc2ccc(Cl)c(Cl)c2)[C@H]2[C@H](N3CCCC3)CCC[C@H]21]

To a solution of (cis,trans)-methyl 5-(pyrrolidin-1-yl)octahydroquinoxaline-1(2H)-carboxylate (54 mg) in dichloromethane (3 ml), a solution of 2-(3,4-dichlorophenyl)acetyl chloride (67.7 mg) in dichloromethane (1 ml) was added. The reaction mixture was stirred at RT overnight and hydrolyzed with 0.5N NaOH (aq.). The mixture was stirred for 30 min and the layers were separated. The organic layer was dried over Na₂SO₄, filtered and concentrated. Purification by flash column chromatography (eluent 1% (7N NH₃ in MeOH)/CH₂Cl₂) afforded 50 mg of Example 1. This batch was combined with another batch (80 mg in total) and purified by chiral prep HPLC to afford 30 mg of one enantiomer, 25 mg of the other enantiomer, and 10 mg of the starting racemic mixture. Conversion to the corresponding HCl salt gave 25 mg of Reference compound A (enantiomer 1) and 20 mg of Example 89 (enantiomer 2).

Reference Compounds F and G

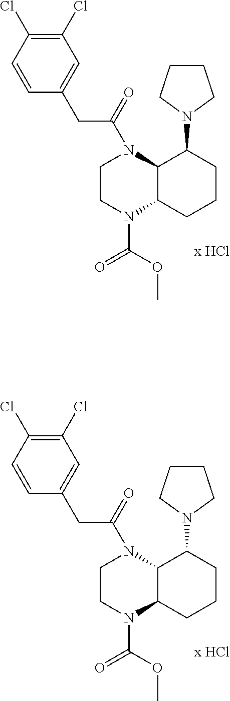 [SMILES: COC(=O)N1CCN(C(=O)Cc2ccc(Cl)c(Cl)c2)[C@H]2[C@@H](N3CCCC3)CCC[C@@H]21 ; COC(=O)N1CCN(C(=O)Cc2ccc(Cl)c(Cl)c2)[C@@H]2[C@H](N3CCCC3)CCC[C@H]21]

To a solution of (trans,cis)-methyl 5-(pyrrolidin-1-yl)octahydroquinoxaline-1(2H)-carboxylate, (cis,cis)-methyl 5-(pyrrolidin-1-yl)octahydroquinoxaline-1(2H)-carboxylate and (cis,trans)-methyl 5-(pyrrolidin-1-yl)octahydroquinoxaline-1(2H)-carboxylate (125 mg) in dichloromethane (5 ml), a solution of 2-(3,4-dichlorophenyl)acetyl chloride (157 mg) in dichloromethane (2.5 ml) was added. The reaction mixture was stirred at RT overnight and hydrolyzed with 0.5 N NaOH (aq.). The mixture was stirred for 30 min and the layers were separated. The organic layer was dried over Na₂SO₄, filtered and concentrated. Purification by flash column chromatography (eluent 1% (7N NH₃ in MeOH)/CH₂Cl₂) afforded crude product (125 mg) as a mixture of Reference compound E, Example 1 and Reference compounds F and G (racemate) (increasing retention time order). LCMS spiking experiments confirmed that Reference compounds F and G (racemate) are a new diastereoisomer. Purification by flash column chromatography (eluent 0.1-0.5% (7N NH₃ in MeOH)/CH₂Cl₂) afforded 46 mg as a mixture of Example 1 and Reference compounds F and G (racemate). Purification by chiral prep-HPLC gave, after concentration, dilution in Et₂O, filtration and concentration, 4 fractions (increasing retention time):

Reference compound F

Reference compound G (contains some Reference compound A)

Reference compound A

Example 89

Reference compounds F and G were converted into the corresponding HCl salt.

Common Intermediates:

### 5,6,7,8-Tetrahydroquinoxaline 1-oxide

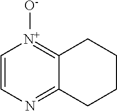 [SMILES: [O-][n+]1ccnc2c1CCCC2]

5,6,7,8-Tetrahydroquinoxaline (250 g) was dissolved in dichloromethane (3 l). The solution was placed under nitrogen, cooled to 3° C. and 3-chloroperbenzoic acid (77%, 482 g) was added in small portions over a time period of 90 min. During addition the reaction mixture was kept below 5° C. When the addition was complete, the reaction mixture had turned into a turbid white slurry and the reaction mixture was then allowed to slowly reach ambient T overnight (18 hours reaction time). At 17° C. 10% Na₂S₂O₃ (aq., 884 ml) was added drop wise to the stirring reaction mixture in 20 min time. A sample from the reaction mixture was checked for peroxides with a wet (water) peroxide strip. Next, sat. NaHCO₃ aq. (2 l) was added to the stirring reaction mixture in 30 min time and the mixture was stirred for an additional 30 min until no more gas evolved from the reaction mixture. The organic layer was divided into two portions and both portions were extracted with sat. NaHCO₃ (aq., 500 ml). The aqueous layer from the reaction mixture was extracted three times with CH₂Cl₂ (1 l) and each CH₂Cl₂ layer was washed with sat. NaHCO₃ (aq., 300 ml). All CH₂Cl₂ layers were combined and dried over Na₂SO₄, filtered and evaporated in vacuo. A sample from the residue was checked for peroxides (sample in CH₂Cl₂ and wet peroxide strip). The residue was co-evaporated with Et₂O and heptane. This afforded the crude product (226.8 g). The crude product was crushed with mortar and pestle and triturated in heptane (480 ml) for 2 hours. The product was filtered off, washed with heptane (200 ml) and dried in vacuo at 50° C. (rotating evaporator). This afforded 187.3 g of the N-oxide.

### (R,S)-5,6,7,8-Tetrahydroquinoxalin-5-ol

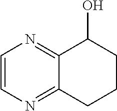 [SMILES: OC1CCCc2nccnc21]

5,6,7,8-Tetrahydroquinoxaline 1-oxide (264.4 g) was dissolved in dichloromethane (2644 ml) and the flask was placed under nitrogen, cooled to 0° C. and trifluoroacetic anhydride (1109 g) was added drop wise in 100 min time, while the temperature was kept below 5° C. Next, the cooling bath under the reaction mixture was slowly allowed to reach 18° C. The reaction mixture was stirred for 17 h at 18° C. (ambient T). The reaction mixture was evaporated in vacuo and stripped with CH₂Cl₂. This afforded 633 g residue (TFA salt of the TFA ester intermediate). The residue was dissolved in dichloromethane (2644 ml) and 2 N lithium hydroxide monohydrate sol. in water (1761 ml) was added drop wise, while keeping the temperature below 20° C. with an acetone dry ice bath. The reaction mixture was stirred for 1 h at ambient T. The reaction mixture was filtered over a layer of diatomaceous earth and sand. The filtrate was left at 19° C. overnight. To the filtrate sat. aq. NaCl (1.5 l) was added and the layers were stirred for 10 min and was then allowed to rest for 30 min. The bottom CH₂Cl₂ layer (·2.5 l) was isolated with a separating funnel, dried over Na₂SO₄, filtered and evaporated in vacuo. This afforded 138.6 g of black oil which slowly solidified. The aqueous layer was extracted three times with EtOAc (1 l). The EtOAc layers were combined, dried over Na₂SO₄, filtered and evaporated in vacuo. This afforded 45.0 g of black oil which slowly solidified. The aqueous layer was extracted four times with EtOAc (1 l). The EtOAc layers were combined, dried over Na₂SO₄, filtered and evaporated in vacuo. This afforded 45.6 g of black oil which slowly solidified. The three batches were combined and used as such in the next step.

### (R,S)-5-((tert-Butyldimethylsilyl)oxy)-5,6,7,8-tetrahydro-quinoxaline

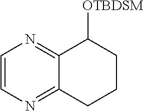

(R,S)-5,6,7,8-tetrahydroquinoxalin-5-ol (9.63 g) was dissolved in dichloromethane (300 ml) and cooled to 0° C. 2,6-Lutidine (8.96 ml) was added followed by drop wise addition of tert-butyldimethylsilyl trifluoromethanesulfonate (17.67 ml) over a 10 minute period. Stirring was continued at 0° C. for 3 hours. The reaction mixture was washed with 300 ml saturated NaHCO₃ (aq.) and the organic layer was dried over Na₂SO₄, filtered and concentrated in vacuo (2,6-lutidine was removed by concentration with an external oil pump). The crude product was coated on Isolute (30 g) and purified by flash column chromatography with 10%-30% EtOAc in heptane as eluent to yield the product (11.6 g) as clear brown oil.

### cis,cis-5-(tert-Butyldimethylsilyloxy)decahydroquinoxaline

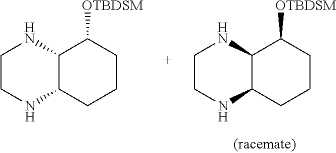

To a nitrogen flushed solution of (R,S)-5-(tert-butyldimethylsilyloxy)-5,6,7,8-tetrahydroquinoxaline (11.6 g) in methanol (150 ml), a slurry of platinum (IV) oxide (1.992 g) in methanol (15 ml) was added. The reaction mixture was placed under 5 bar H₂ pressure (in a glass hydrogenation autoclave) and was stirred at 50° C. for 68 hours. GCMS-analysis showed 39% starting material and 53% desired product. To the (nitrogen flushed) reaction mixture, platinum (IV) oxide (1.494 g) was added (as a slurry in 10 ml MeOH). The reaction was continued under 5 bar H₂ pressure and at 50° C. for another 22 hours, after which GCMS-analysis showed 15% starting material remained. Once again platinum (IV) oxide (280 mg) was added (as a slurry in 3 ml MeOH) and the reaction was placed under 5 bar H₂ pressure and stirred at 50° C. for 23 hours, after which GCMS-analysis showed complete conversion. The reaction mixture was filtered over diatomaceous earth and the filtrate was evaporated in vacuo. This afforded 11.3 g product, which was used as such in the next step.

### cis,cis-tert-Butyl 5-(tert-butyldimethylsilyloxy)octahydroquinoxaline-1(2H)-carboxylate

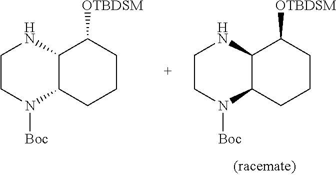

To a solution of cis,cis-5-(tert-butyldimethylsilyloxy)decahydroquinoxaline (11.3 g) in dichloromethane (250 ml) di-tert-butyl dicarbonate (9.57 g) was added. The reaction mixture was stirred at RT overnight. After 18 h, the reaction mixture was diluted with 150 ml CH₂Cl₂ and washed with 150 ml water (2×). The CH₂Cl₂ layer was dried over Na₂SO₄, filtered and evaporated in vacuo. The crude material was purified by column chromatography to yield 13.9 g product.

### cis,cis-tert-Butyl 5-hydroxyoctahydroquinoxaline-1(2H)-carboxylate

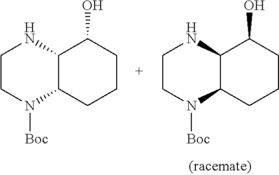

To a solution of cis,cis-tert-butyl 5-(tert-butyldimethylsilyloxy)octahydroquinoxaline-1(2H)-carboxylate (13.9 g) in methanol (350 ml) ammonium fluoride (20.84 g) was added. The solution was kept under reflux conditions for 20 hours. To the reaction mixture 350 ml sat. Na₂CO₃ (aq.) was added (pH>10), after which MeOH was evaporated in vacuo. The alkaline aqueous solution was extracted with EtOAc (3×). The combined EtOAc layers were dried over Na₂SO₄, filtered and evaporated in vacuo (1× coevaporated with CH₂Cl₂). This afforded 10 g crude product, which was further purified by gravity column chromatography (10% MeOH in CH₂Cl₂). This afforded 7.47 g product that was used as such in the next step.

### tert-Butyl(6aSR,9aRS,9bSR)octahydro-6H-[1,2,3]oxathiazolo[3,4,5-de]quinoxaline-6-carboxylate 2,2-dioxide

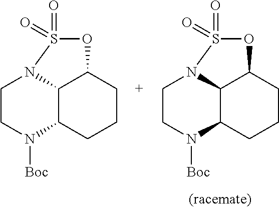

At 0° C. a solution of sulfuryl chloride (2.60 ml) in dichloromethane (125 ml) was added to a solution of cis,cis-tert-butyl 5-hydroxyoctahydroquinoxaline-1(2H)-carboxylate (7.47 g) and triethyl amine (11.18 ml) in dichloromethane (250 ml). The reaction mixture was slowly allowed to reach RT and stirred for 20 h. The reaction mixture was washed with 150 ml sat. NaHCO₃ (aq.) and 100 ml water. The organic layer was dried over Na₂SO₄, filtered and evaporated in vacuo. This afforded 8.64 g crude product, which was further purified by flash column chromatography (30% EtOAc in heptane) to yield 5.53 g product, which was used as such in the next step.

### (4aRS,5SR,8aSR)-tert-Butyl-5-(pyrrolidin-1-yl)octahydroquinoxaline-1(2H)-carboxylate

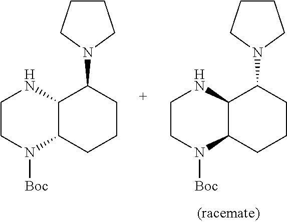 [SMILES: CC(C)(C)OC(=O)N1CCN[C@@H]2[C@@H](N3CCCC3)CCC[C@@H]21 ; CC(C)(C)OC(=O)N1CCN[C@H]2[C@H](N3CCCC3)CCC[C@H]21]

A mixture of (31RS,6aRS,9aSR)-tert-butyl hexahydro-31H-[1,2,3]oxathiazolo[3,4,5-de]-quinoxaline-6(6aH)-carboxylate 2,2-dioxide (1.91 g) and pyrrolidine (1.478 ml) in anhydrous acetonitrile (50 ml) was stirred at 70° C. for 22 hours. The reaction mixture was evaporated in vacuo, coevaporated with toluene and CH₂Cl₂ (removal excess pyrrolidine). The residue was taken up in CH₂Cl₂, 50 ml 10% citric acid (aq.) was added and the mixture was shaken for 2 min, after which the layers were separated. The acidic aqueous layer was basified with 1N NaOH (aq.) and extracted with CH₂Cl₂ (2×50 ml). The combined CH₂Cl₂ extracts were dried over Na₂SO₄, filtered and evaporated in vacuo. This afforded 1.92 g product, which was used as such in the next step.

### cis,cis-1-Benzyl-5-(tert-butyldimethylsilyloxy)decahydroquinoxaline

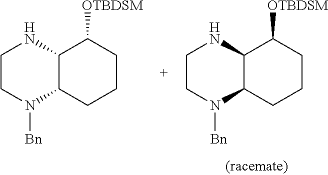

To a solution of cis,cis-5-(tert-butyldimethylsilyloxy)decahydroquinoxaline (3.0 g) in dry N,N-dimethylformamide (105 ml) potassium carbonate (3.07 g) and benzyl bromide (1.393 ml) were added. The reaction mixture was stirred at 80° C. for 1 h. The reaction mixture was evaporated in vacuo. The residue was dissolved in EtOAc, washed with water and brine and dried over Na₂SO₄, filtered and concentrated in vacuo. The crude material was purified by flash column chromatography (5% MeOH in CH₂Cl₂) to yield 2.65 g of product.

### cis,cis-1-Benzyldecahydroquinoxalin-5-ol

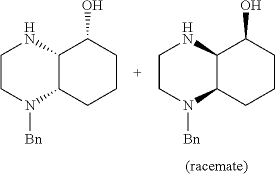

To a solution of cis,cis-1-benzyl-5-(tert-butyldimethylsilyloxy)decahydroquinoxaline (2.65 g) in methanol (extra dry, 80 ml) ammonium fluoride (4.08 g) was added. The reaction mixture was kept under reflux conditions for 20 h. Saturated Na₂CO₃ (aq.) was added and the mixture was evaporated in vacuo (coevaporated 4× with MeOH). The solid residue was triturated (3×) with 100 ml CH₂Cl₂. The combined CH₂Cl₂ filtrates were dried over Na₂SO₄, filtered and concentrated in vacuo. This afforded 1.81 g product. The product was coevaporated once with CH₂Cl₂ to remove Et₂O and was used as such in the subsequent step.

### (6aSR,9aRS,9bSR)-6-Benzyloctahydro-4H-[1,2,3]oxathiazolo[3,4,5-de]quinoxaline 2,2-dioxide

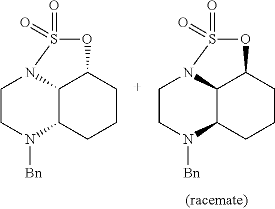

The reaction was performed in the dark. A solution of sulfuryl chloride (0.591 ml) in dichloromethane (20 ml) was added dropwise to a solution of cis,cis-1-benzyldecahydroquinoxalin-5-ol (1.8 g) and triethylamine (3.05 ml) in dichloromethane (60 ml) at 0° C. The solution was stirred at 0° C. for 1 h and at RT for 4 h. The mixture was partially concentrated at 35° C., filtered and immediately purified by flash chromatography (EtOAc/heptane 1:1) to afford 442 mg product which was used right away for the next step.

### (4aRS,5SR,8aSR)-1-Benzyl-5-(pyrrolidin-1-yl)decahydroquinoxaline

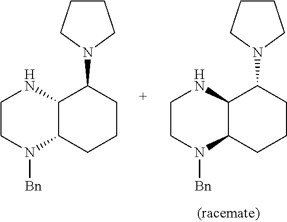 [SMILES: c1ccc(CN2CCN[C@@H]3[C@@H](N4CCCC4)CCC[C@@H]32)cc1 ; c1ccc(CN2CCN[C@H]3[C@H](N4CCCC4)CCC[C@H]32)cc1]

Pyrrolidine (0.589 ml) was added to a solution of (31RS,6aRS,9aSR)-6-benzyloctahydro-31H-[1,2,3]oxathiazolo[3,4,5-de]quinoxaline 2,2-dioxide (442 mg) in anhydrous acetonitrile (10 ml) and the solution was stirred at 70° C. for 20 h. The mixture was concentrated in vacuo, 10 ml 1M HCl (aq.) was added and the mixture was stirred at 50° C. for 1 h. The acidic aqueous layer was washed with Et₂O and basified with 2N NaOH (aq.). The basic aqueous layer was extracted with dichloromethane. The organic layer was dried over Na₂SO₄, filtered and concentrated to afford the crude product. The residue was triturated in Et₂O, filtered and the filtrate was concentrated to give 360 mg product, which was used as such in the next step.

### cis,cis-Benzyl 5-(tert-butyldimethylsilyloxy)octahydroquinoxaline-1(2H)-carboxylate

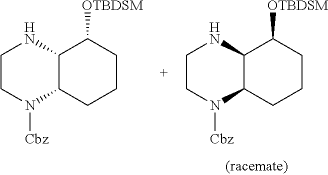

Benzyl chloroformate (0.110 ml) was added to a solution of cis,cis-5-(tert-butyldimethylsilyloxy)decahydroquinoxaline (200 mg) in dichloromethane (4 ml) and the reaction mixture was stirred at RT for 2 h. The reaction mixture was diluted with DCM, washed with sat. Na₂CO₃ (aq.), dried over Na₂SO₄, filtered and concentrated. The residue was triturated in heptane, filtered and concentrated to afford 181 mg product.

### cis,cis-Benzyl 5-hydroxyoctahydroquinoxaline-1(2H)-carboxylate

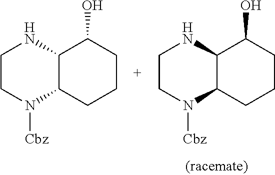

Ammonium fluoride (249 mg) was added to a solution of cis,cis-benzyl 5-(tert-butyldimethylsilyloxy)octahydroquinoxaline-1(2H)-carboxylate (181 mg, 0.447 mmol) in methanol (extra dry, 5 ml) and the mixture was stirred under reflux conditions overnight. The reaction mixture was concentrated; the residue was taken in CH₂Cl₂ and sat. Na₂CO₃ (aq.) was added. After shaking, the biphasic mixture was concentrated; the residue was taken up in CH₂Cl₂, dried over Na₂SO₄, filtered and concentrated to afford 110 mg of product.

### Benzyl (6aSR,9aRS,9bSR)octahydro-6H-[1,2,3]oxathiazolo[3,4,5-de]quinoxaline-6-carboxylate 2,2-dioxide

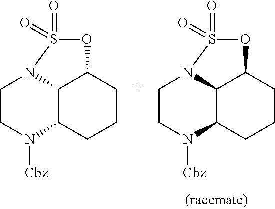

A solution of sulfuryl chloride (0.032 ml) in dichloromethane (1 ml) was added dropwise to a solution of cis,cis-benzyl 5-hydroxyoctahydroquinoxaline-1(2H)-carboxylate (110 mg) and triethylamine (0.158 ml) in dichloromethane (3 ml) at 0° C. The solution was stirred at 0° C. for 1 h and at RT for 1 h. The mixture was diluted with CH₂Cl₂, hydrolysed with water and the organic layer was dried over Na₂SO₄, filtered and concentrated. Purification by flash chromatography (EtOAc/heptane 1:1) yielded 35 mg of product.

### (4aRS,5SR,8aSR)-Benzyl-5-(pyrrolidin-1-yl)octahydroquinoxaline-1(2H)-carboxylate

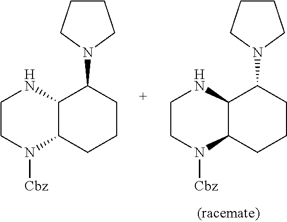 [SMILES: O=C(OCc1ccccc1)N1CCN[C@@H]2[C@@H](N3CCCC3)CCC[C@@H]21 ; O=C(OCc1ccccc1)N1CCN[C@H]2[C@H](N3CCCC3)CCC[C@H]21]

Pyrrolidine (0.024 ml) was added to a solution of (31RS,6aRS,9aSR)-benzyl hexahydro-31H-[1,2,3]oxathiazolo[3,4,5-de]quinoxaline-6(6aH)-carboxylate 2,2-dioxide (35 mg) in anhydrous acetonitrile (1 ml) and the solution was stirred at 70° C. for 20 h. The mixture was concentrated in vacuo, the residue was taken up in CH₂Cl₂, washed (after thorough shaking) with 10% aqueous citric acid solution, dried over Na₂SO₄, filtered and concentrated to yield 34 mg of product.

### 7,8-Dihydroquinoxalin-5(6H)-one

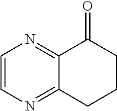 [SMILES: O=C1CCCc2nccnc21]

A solution of crude 5,6,7,8-tetrahydroquinoxalin-5-ol (183.6 g, 50%) in dichloromethane (2000 ml) was cooled to 5° C. and Dess-Martin periodinane (solid) (298 g) was added slowly in portions in 15 min. time, keeping the temperature between 5° C. and 10° C. Next a mixture of water (12.66 g) and dichloromethane (4000 ml) was added drop wise in 30 min, keeping the temperature between 5-10° C. The temperature in the cooling bath was slowly allowed to reach ambient T. The reaction was stirred over night at ambient T (16 h). To the reaction methanol (124 ml) was added drop wise and the reaction mixture was stirred at RT for 0.5 h. The reaction mixture was filtered over a plug of 1 kg silica (˜2 liter). The filter was rinsed with 5% MeOH in CH₂Cl₂ (5×1 l). The filtrates were combined and evaporated in vacuo. The crude material was purified by gravity column chromatography (silica gel, eluent: 100% EtOAc).

### (R)-5,6,7,8-Tetrahydroquinoxalin-5-ol

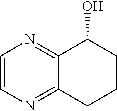 [SMILES: O[C@@H]1CCCc2nccnc21]

7,8-Dihydroquinoxalin-5(6H)-one (106.9 g), dichloro(p-cymene)ruthenium(II)dimer (2.209 g) and (1R,2R)-N-p-tosyl-1,2-diphenylethylenediamine (2.64 g) were placed in a 2 l 3-neck flask. The flask was placed under nitrogen. Next, nitrogen flushed N,N-dimethylformamide (700 ml) was added followed by the drop wise addition of triethylammonium formate 2:5 (74.9 g). The reaction mixture was stirred at 20° C. (ambient T) for 4 hours and evaporated in vacuo. This afforded 129.9 g crude product. The crude product was dissolved in EtOAc (250 ml) and filtered (1 l P3 glass filter with 1 cm sand and silica (125 g)). The silica was flushed 3× with EtOAc (500 ml each) and the filtrate was evaporated in vacuo (1× co-evaporation with CH₂Cl₂). This afforded 115.8 g crude product with an enantiomeric excess of 98.4% (R). The material was used as such in the next step.

### (R)-5-((tert-Butyldimethylsilyl)oxy)-5.6.7.8-tetrahydro-quinoxaline

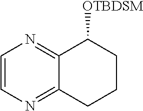

Under nitrogen, a solution of (R)-5,6,7,8-tetrahydroquinoxalin-5-ol (115.8 g, 86%) and 2,6-lutidine (85 g) in dichloromethane (600 ml) was cooled to 5-10° C. To the reaction mixture tert-butyldimethylsilyl trifluoromethanesulfonate (210 g) was added drop wise in 20 min while keeping the temperature below 10° C. The reaction mixture was washed twice with sat. aq. NaHCO₃ (250 ml each), dried over Na₂SO₄, filtered and evaporated in vacuo. The crude product was purified by gravity column chromatography (column diameter 16 cm, 1.5 kg silica, eluent 25% EtOAc in heptane). This afforded the product as brown clear liquid oil.

### (4aS,5R,8aS)-5-((tert-Butyldimethylsilyl)oxy)-decahydroquinoxaline acetate

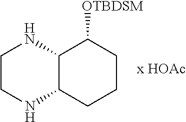

The experiment was performed in a 4 liter autoclave at 50° C. under a 5 bar hydrogen atmosphere. To a solution of (R)-5-((tert-butyldimethylsilyl)oxy)-5,6,7,8-tetrahydro-quinoxaline (204.5 g) in methanol (1.5 l), acetic acid (0.045 l) and platinum (IV) oxide (8.78 g) was added. The reaction mixture was flushed twice with hydrogen without stirring and once with stirring and was then placed under a 5 bar hydrogen atmosphere. The reaction mixture was brought to 50° C. in 45-60 min. During this period the pressure was kept on 5 bar hydrogen pressure (rapid hydrogen consumption). At 50° C. it took another 60 minutes before the reaction mixture remained on 5 bar hydrogen pressure. The reaction mixture was stirred an additional 60 min at 50° C. The reaction mixture was then flushed with nitrogen and filtered over diatomaceous earth and partly evaporated in vacuo and was stored overnight under nitrogen at 18° C. The reaction mixture was further evaporated in vacuo and co-evaporated with CH₂Cl₂. This afforded the crude product (254.0 g) as a brown clear gel. The product was used as such in the next step.

### (4aS,5R,8aS)-Methyl 5-((tert-butyldimethylsilyl)oxy)octahydroquinoxaline-1(2H)-carboxylate

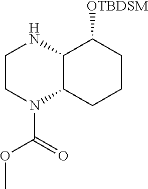

The experiment was performed under nitrogen atmosphere in a 4 liter 3-neck flask, with a magnetic stirring bar. To an ice/water cooled solution of (4aS,5R,8aS)-5-((tert-butyldimethylsilyl)oxy)-decahydroquinoxaline acetate (253 g, 97%) in dichloromethane (1125 ml), triethylamine (117 ml) was added drop wise and a solution of methyl chloroformate (57.5 ml) in dichloromethane (125 ml) was also added drop wise. The reaction mixture was stirred at RT for 1 hour. The reaction mixture was washed with sat. aq. NaHCO₃ (1250 ml) and water (500 ml). The CH₂Cl₂ layer was dried over Na₂SO₄, filtered and evaporated in vacuo. This afforded the crude product (245.1) as brown clear oil. The product was used as such in the next step.

### (4aS,5R,8aS)-Methyl-5-hydroxyoctahydroquinoxaline-1(2H)-carboxylate

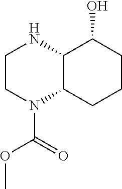

The experiment was performed under nitrogen atmosphere in a 4 l 3-neck flask equipped with a magnetic stirring bar and a water cooler. Under a nitrogen atmosphere ammonium fluoride (392 g) was added to a solution of (4aS,5R,8aS)-methyl 5-((tert-butyldimethylsilyl)oxy)octahydro-quinoxaline-1(2H)-carboxylate (245.1 g) in methanol (2500 ml). The reaction mixture was kept under reflux conditions for 40 hours. To the reaction mixture sat. aq. Na₂CO₃ (1 l) was added and the reaction mixture was evaporated in vacuo. To the sticky solid residue CH₂Cl₂ was added, stirred and the salts were filtered off. This was repeated 4 times with 1 l CH₂Cl₂ each. The filtrates were combined dried over Na₂SO₄ and evaporated in vacuo to afford the crude product (76.7 g). To the salts sat. Na₂CO₃ (500 ml) was added and almost immediately an oily brown organic product floated on the aqueous suspension. This mixture was extracted with CH₂Cl₂ (4×500 ml). The combined layers were dried over Na₂SO₄ and evaporated in vacuo to afford a second batch of crude product (86.2 g). The two batches were combined and further dried in vacuo to yield 149.6 g product.

### Methyl (6aS,9aR,9bS)octahydro-6H-[1,2,3]oxathiazolo[3,4,5-de]quinoxaline-6-carboxylate 2,2-dioxide

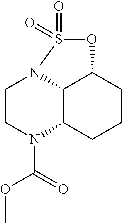 [SMILES: COC(=O)N1CCN2[C@@H]3[C@@H](CCC[C@@H]31)OS2(=O)=O]

The experiment was performed under nitrogen atmosphere in a 4 l 3-neck flask, magnetic stirrer and equipped with a digital thermometer. A solution of sulfuryl chloride (54.2 ml) in dichloromethane (750 ml) was added drop wise to an ice-water cooled solution of (4aS,5R,8aS)-methyl-5-hydroxyoctahydroquinoxaline-1(2H)-carboxylate (149.6 g, 80%) and triethylamine (233 ml) in dichloromethane (1500 ml), at such rate that the temperature in the reaction flask did not exceed 6° C. After 60 min the addition was complete and the reaction mixture was left stirring while the cooling bath was allowed to reach ambient T. After 16 h the reaction mixture was washed three times with a NaHCO₃ solution in water (500 ml sat. aq. NaHCO₃ in 500 ml water). The CH₂Cl₂ layer was dried over Na₂SO₄, filtered and evaporated in vacuo. This afforded 169 g crude product. The material was further purified by column chromatography (2.5 kg silica, eluent: heptane/EtOAc, 1:1) to afforded the product (102.2 g).

### (4aR,5S,8aS)-Methyl-5-(pyrrolidin-1-yl)octahydroquinoxaline-1(2H)-carboxylate

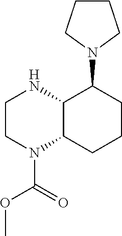 [SMILES: COC(=O)N1CCN[C@@H]2[C@@H](N3CCCC3)CCC[C@@H]21]

The experiment was performed under nitrogen in a 1 l 3-neck flask with magnetic stirrer, a digital thermometer and water cooler attached. A mixture of (31S,6aS,9aR)-methyl hexahydro-31H-[1,2,3 ]oxathiazolo[3,4,5-de]quinoxaline-6(6aH)-carboxylate 2,2-dioxide (50 g) and pyrrolidine (74.3 ml) in acetonitrile (anhydrous) (250 ml), was refluxed at 80° C. for 18 hours. The reaction mixture was evaporated in vacuo (co-evaporation with toluene and CH₂Cl₂). The residue (brown clear oil) was dissolved in 1N HCl aq. (500 ml) and washed twice with Et₂O (250 ml each). The acidic aqueous layer was made alkaline with 2N NaOH aq. (˜250 ml) and the brown alkaline aqueous layer was extracted three times with Et₂O (500 ml each). The Et₂O layers were combined, dried over Na₂SO₄, filtered and evaporated in vacuo to afford the crude product (37.1 g). The alkaline brown clear aqueous layer was extracted twice with Et₂O (500 ml each). The Et₂O layers were combined, dried over Na₂SO₄, filtered and evaporated in vacuo. This afforded a second batch of product (4.3 g). The alkaline brown clear aqueous layer was then saturated with NaCl and extracted Et₂O (500 ml). The Et₂O layer was dried over Na₂SO₄, filtered and evaporated in vacuo. This afforded a third batch of product (1.6 g). The three batches were dissolved in CH₂Cl₂, combined and evaporated in vacuo. This afforded 44.7 g of product.

### (4aS,5R,8aS)-tert-Butyl 5-((tert-butyldimethylsilyl)oxy)octahydroquinoxaline-1(2H)-carboxylate

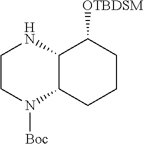

To an ice/water cooled solution of (4aS,5R,8aS)-5-((tert-butyldimethylsilyl)oxy)deca-hydroquinoxaline acetate (9.824 g) in dichloromethane (90 ml), triethylamine (3.46 g) was added drop wise, followed by drop wise addition of a solution of di-tert-butyl dicarbonate (6.54 g) in dichloromethane (12 ml). The reaction mixture was stirred at RT for 3 h and washed with water (2×). The organic layer was dried over Na₂SO₄ and concentrated in vacuo. The crude material was purified by gravity column chromatography (0-2.5% MeOH/DCM) to yield 11.22 g product.

### (4aS,5R,8aS)-tert-Butyl-5-hydroxyoctahydroquinoxaline-1(2H)-carboxylate

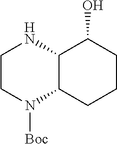 [SMILES: CC(C)(C)OC(=O)N1CCN[C@@H]2[C@H](O)CCC[C@@H]21]

Under nitrogen atmosphere ammonium fluoride (17.02 g) was added to a solution of (4aS,5R,8aS)-tert-butyl 5-((tert-butyldimethylsilyl)oxy)octahydroquinoxaline-1(2H)-carboxylate (12 g) in methanol (125 ml). The reaction mixture was kept under reflux conditions for 23 hours. The reaction mixture was cooled to RT and filtered. The filtrate was concentrated in vacuo, 60 ml of sat. Na₂CO₃ (aq.) was added and traces of MeOH were removed in vacuo. The aqueous phase was extracted with CH₂Cl₂ (4×30 ml). The combined organic phases were dried over Na₂SO₄ and concentrated in vacuo. The crude product (7.8 g) was used as such for the next step.

### tert-Butyl(6aS,9aR,9bS)octahydro-6H-[1,2,3]oxathiazolo[3,4,5-de]quinoxaline-6-carboxylate 2,2-dioxide

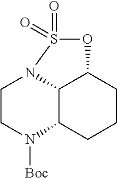 [SMILES: CC(C)(C)OC(=O)N1CCN2[C@@H]3[C@@H](CCC[C@@H]31)OS2(=O)=O]

A solution of sulfuryl chloride (3.82 g) in dichloromethane (30 ml) was added dropwise to an ice/water cooled solution of (4aS,5R,8aS)-tert-butyl 5-hydroxyoctahydroquinoxaline-1(2H)-carboxylate (6.05 g) and triethylamine (7.16 g) in dichloromethane (60 ml), at such rate that the temperature in the reaction flask did not exceed 6° C. When addition was complete, the reaction mixture was left stirring while the cooling bath was allowed to reach RT. The reaction mixture was stirred overnight and washed 3× with NaHCO₃ solution in water (35 ml sat. NaHCO₃ (aq.) in 35 ml water). The organic layer was dried over Na₂SO₄ and concentrated in vacuo. The crude product was purified by flash column chromatography (0-40% EtOAc/heptane). The product was obtained as yellow oil (3.7 g) which solidified upon standing.

### (4aR,5S,8aS)-tert-Butyl-5-((S)-3-hydoxypyrrolidin-1-yl)octahydroquinoxaline-1(2H)-carboxylate

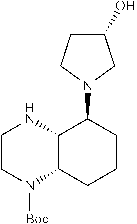 [SMILES: CC(C)(C)OC(=O)N1CCN[C@@H]2[C@@H](N3CC[C@H](O)C3)CCC[C@@H]21]

(S)-3-Pyrrolidinol (2.175 g) and potassium carbonate (0.138 g) were added to a solution of (31 S,6aS,9aR)-tert-butyl hexahydro-31H-[1,2,3 ]oxathiazolo[3,4,5-de]quinoxaline-6(6aH)-carboxylate 2,2-dioxide (1.59 g) in dry N,N-dimethylformamide (4 ml) and the solution was stirred at 70° C. for 2 days. The reaction mixture was concentrated in vacuo, resuspended in Et₂O and extracted with 10% citric acid (aq.). The acidic aqueous layer was washed with Et₂O and basified with 2N NaOH (aq.). The basic aqueous layer was extracted with EtOAc (3×). The combined EtOAc phases were dried over Na₂SO₄ and concentrated to give 2.1 g crude product. Purified by flash column chromatography (1-5% (7N NH₃ in MeOH)/CH₂Cl₂) yielded 1.61 g product as yellow oil, which solidified upon standing.

### 2-(3,4-Dichlorophenyl)acetyl chloride

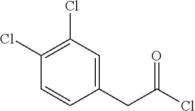 [SMILES: O=C(Cl)Cc1ccc(Cl)c(Cl)c1]

To a solution of 3,4-dichlorophenylacetic acid (400 mg) in dry diethyl ether (12 ml), N,N-dimethylformamide (catalytic) and oxalyl chloride (0.184 ml) were added. The reaction mixture was stirred at RT for 2 h, concentrated, coevaporated with dichloromethane (2×) to afford 2-(3,4-dichlorophenyl)acetyl chloride. The product was used as such in the next step.

### Synthesis of Example 15

### Example 15

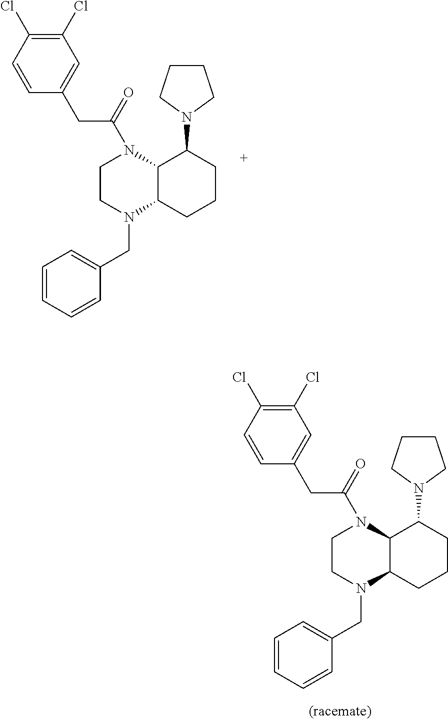 [SMILES: O=C(Cc1ccc(Cl)c(Cl)c1)N1CCN(Cc2ccccc2)[C@H]2CCC[C@H](N3CCCC3)[C@H]21 ; O=C(Cc1ccc(Cl)c(Cl)c1)N1CCN(Cc2ccccc2)[C@@H]2CCC[C@@H](N3CCCC3)[C@@H]21]

A solution of 2-(3,4-dichlorophenyl)acetyl chloride (403 mg) in dichloromethane (2 ml) was added to a solution of (4aRS,5 SR, 8aSR)-1-benzyl-5-(pyrrolidin-1-yl)decahydroquinoxaline (360 mg) in dichloromethane (6 ml) at RT and the reaction mixture was stirred at RT overnight. The reaction mixture was diluted with dichloromethane and hydrolysed with water. The aqueous layer was basified with 0.5M NaOH (aq.). The organic layer was dried over Na₂SO₄, filtered and concentrated in vacuo. Purification by flash chromatography (eluent CH₂Cl₂/3-10% MeOH) yielded 460 mg product.

### Synthesis of Example 16

### Example 16

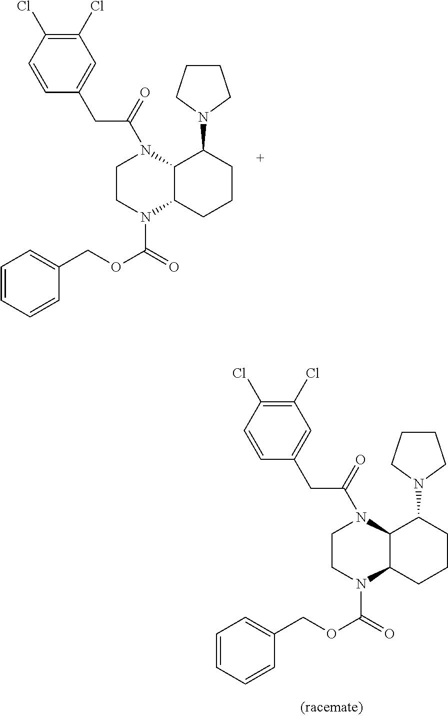 [SMILES: O=C(Cc1ccc(Cl)c(Cl)c1)N1CCN(C(=O)OCc2ccccc2)[C@H]2CCC[C@H](N3CCCC3)[C@H]21 ; O=C(Cc1ccc(Cl)c(Cl)c1)N1CCN(C(=O)OCc2ccccc2)[C@@H]2CCC[C@@H](N3CCCC3)[C@@H]21]

A solution of 2-(3,4-dichlorophenyl)acetyl chloride (31.2 mg) in dichloromethane (1 ml) was added to a solution of (4aRS,5SR,8aSR)-benzyl-5-(pyrrolidin-1-yl)octahydroquinoxaline-1(2H)-carboxylate (32 mg) and N,N-diisopropylethylamine (0.032 ml) in dichloromethane (2 ml) at RT. The reaction mixture was stirred at RT overnight. The reaction mixture was diluted with CH₂Cl₂ and hydrolysed with water. The aqueous layer was basified with 0.5 M NaOH (aq.), the organic layer was dried over Na₂SO₄, filtered and concentrated in vacuo. Purification by flash column chromatography (eluent CH₂Cl₂/5-10% MeOH) followed by trituration in Et₂O provided the final product.

### Synthesis of Example 24

### Example 24

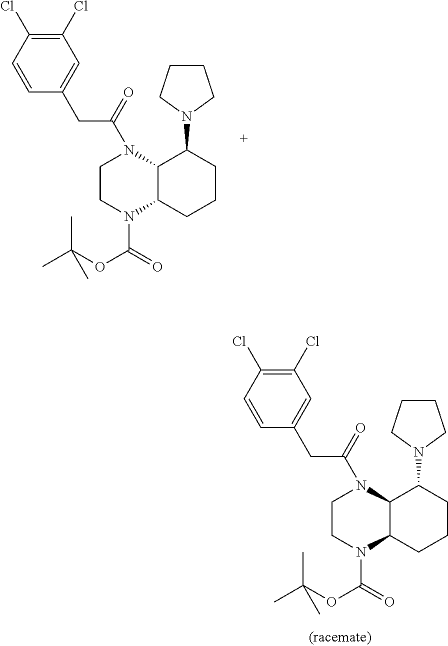 [SMILES: CC(C)(C)OC(=O)N1CCN(C(=O)Cc2ccc(Cl)c(Cl)c2)[C@@H]2[C@@H](N3CCCC3)CCC[C@@H]21 ; CC(C)(C)OC(=O)N1CCN(C(=O)Cc2ccc(Cl)c(Cl)c2)[C@H]2[C@H](N3CCCC3)CCC[C@H]21]

To a solution of (4aRS,5SR,8aSR)-tert-butyl-5-(pyrrolidin-1-yl)octahydroquinoxaline-1(2H)-carboxylate (1.92 g) and N,N-diisopropylethylamine (2.124 ml) in dichloromethane (160 ml), a solution of 2-(3,4-dichlorophenyl)acetyl chloride (2.080 g) in dichloromethane (80 ml) was added in 30-45 min. The reaction mixture was stirred at RT for 1 h. The reaction mixture was washed with 2×50 ml 0.5N NaOH (aq.). The organic layer was dried over Na₂SO₄, filtered and evaporated in vacuo. The crude product was purified by flash chromatography (1% MeOH (7N NH₃) in CH₂Cl₂).

### Synthesis of Example 39

### Example 39

### Preparation 1

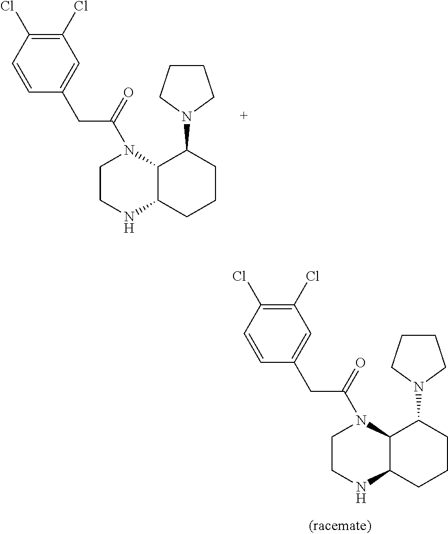 [SMILES: O=C(Cc1ccc(Cl)c(Cl)c1)N1CCN[C@H]2CCC[C@H](N3CCCC3)[C@H]21 ; O=C(Cc1ccc(Cl)c(Cl)c1)N1CCN[C@@H]2CCC[C@@H](N3CCCC3)[C@@H]21]

To a solution of Example 24 (527 mg) in dichloromethane (5 ml), trifluoroacetic acid (2.358 ml) was added. The reaction mixture was stirred at RT overnight. The reaction mixture was evaporated in vacuo and coevaporated with toluene and with CH₂Cl₂ (2×). The residue was dissolved in CH₂Cl₂ and washed with 0.5N NaOH (aq.) and water. The CH₂Cl₂ layer was dried over Na₂SO₄, filtered and evaporated in vacuo.

### Example 39

### Preparation 2

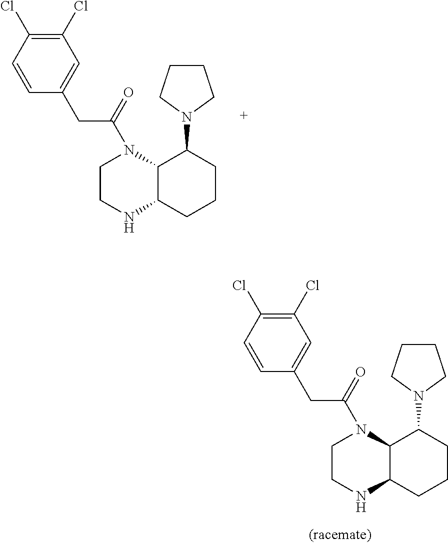 [SMILES: O=C(Cc1ccc(Cl)c(Cl)c1)N1CCN[C@H]2CCC[C@H](N3CCCC3)[C@H]21 ; O=C(Cc1ccc(Cl)c(Cl)c1)N1CCN[C@@H]2CCC[C@@H](N3CCCC3)[C@@H]21]

Concentrated HCl (36% in H₂O, 8 ml) and palladium, 10% on activated carbon (150 mg) were added to a degassed solution of Example 15 (380 mg) in tetrahydrofuran (40 ml) and water (40 ml). The mixture was stirred under H₂ atmosphere (balloon, 1 bar) at RT for 4 h. Extra palladium, 10% on activated carbon (150 mg) was added and the stirring was continued under 1 bar H₂ atmosphere for 1 h. The mixture was filtered and partially concentrated to remove THF. The acidic water layer was washed with Et₂O, basified with 1M NaOH (aq.) and extracted with CH₂Cl₂. The organic layer was dried over Na₂SO₄, filtered and concentrated. The crude product was purified by flash chromatography.

### Synthesis of Example 89

### Example 89

### Free Base

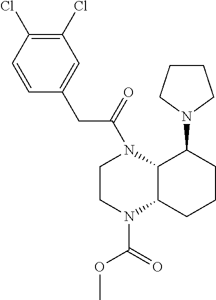 [SMILES: COC(=O)N1CCN(C(=O)Cc2ccc(Cl)c(Cl)c2)[C@@H]2[C@@H](N3CCCC3)CCC[C@@H]21]

The experiment was performed under nitrogen atmosphere in a 2 l 3-neck reaction flask equipped with a digital thermometer and magnetic stirring bar. The reaction was cooled with an ice-water bath. A solution of (4aR,5S,8aS)-methyl-5-(pyrrolidin-1-yl)octahydroquinoxaline-1(2H)-carboxylate (36.5 g, 93%) in dichloromethane (750 ml) was cooled to 0° C. and a solution of 2-(3,4-dichlorophenyl)acetyl chloride (35.8 g, 95%) in dichloromethane (365 ml) was added drop wise while keeping the temperature between 0-2° C. in 105 min. time. When the addition was complete the reaction mixture was stirred at 0-3° C. for additional 30 min and then the cooling bath was removed and the reaction mixture was stirred for another 30 min. at ambient T. The reaction mixture was washed twice with 0.5N NaOH aq. (250 ml each). The organic layer was dried over Na₂SO₄, filtered and evaporated in vacuo. This afforded 56.8 g of the crude product. The crude product (55.8 g) was dissolved in CH₂Cl₂ and further purified by gravity column chromatography (2 kg silica gel, gradient from 0.5% to 1% 7N NH₃ in MeOH in CH₂Cl₂). This afforded three batches of product; 4.3 g (˜90% purity LC-MS), 4.4 g (>95% purity LC-MS) and 43.4 g (>95% purity LC-MS). The purity of the major batch was 98.8% (chiral LC) and 97.6% ee (R).

### Example 89

### Salt

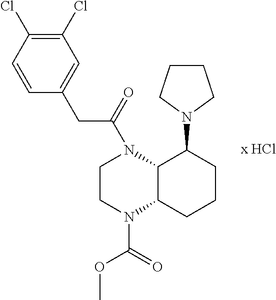 [SMILES: COC(=O)N1CCN(C(=O)Cc2ccc(Cl)c(Cl)c2)[C@@H]2[C@@H](N3CCCC3)CCC[C@@H]21]

The experiment was performed under nitrogen atmosphere in a 1 l reaction flask equipped with a magnetic stirring bar. (4aR,5S,8aS)-methyl 4-(2-(3,4-dichlorophenyl)acetyl)-5-(pyrrolidin-1-yl)octahydroquinoxaline-1(2H)-carboxylate (12 g) was dissolved in dichloromethane (200 ml), cooled with ice/water bath and hydrochloric acid, 1N solution in diethylether (50 ml) was added. The mixture was stirred for 15 min and was then evaporated in vacuo. The residue, which was crushed into a fine solid with a spatula, was co-evaporated twice with Et₂O and then the powder was triturated in Et₂O (100 ml) for 30 min. The Et₂O was decanted and the residue was dried in vacuo on a rotating evaporator at 50° C. for at least 8 h and >96 hours under vacuo (rotary vane pump) at ambient T. The product was dissolved in absolute ethanol (120 ml) in a 500 ml flask on the rotating evaporator at 40° C. When all material was dissolved (after ca. 10 min) vacuum was applied and the mixture was concentrated to dryness to give a yellow foam. A 3-stage membrane pump was fitted to the rotating evaporator and the material was further dried for 1 h, with intermediate grinding of the solids. The material was dissolved in demineralized water (150 ml) and freeze-dried to give an off-white powder (11.7 g) with a purity of 99.0% (chiral LC) and 98.0% ee (R).

### Synthesis of Example 90

Intermediate 90a):

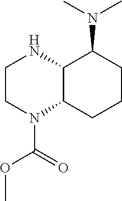 [SMILES: COC(=O)N1CCN[C@@H]2[C@@H](N(C)C)CCC[C@@H]21]

Dimethylamine 2M in THF (1.357 ml) was added to a solution of (31S,6aS,9aR)-methyl hexahydro-31H-[1,2,3 ]oxathiazolo[3,4,5-de]quinoxaline-6(6aH)-carboxylate 2,2-dioxide (250 mg) and potassium carbonate (25.01 mg) in dry N,N-dimethylformamide (4 ml). The solution was stirred in a closed vial at 70° C. for 24 h, after which the reaction mixture was allowed to cool down to RT overnight. The reaction mixture was concentrated, diluted with EtOAc and washed with 10% citric acid (aq.). The aqueous phase was basified with 1N NaOH (aq.) and extracted with EtOAc (2×). The combined organic layer was dried over Na₂SO₄ and concentrated in vacuo affording 364 mg of product as yellow oil. The product was used as such in the next step.

### Example 90

### Free Base

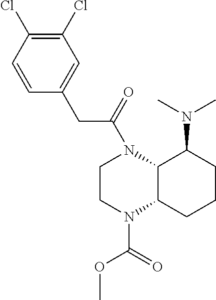 [SMILES: COC(=O)N1CCN(C(=O)Cc2ccc(Cl)c(Cl)c2)[C@@H]2[C@@H](N(C)C)CCC[C@@H]21]

To a solution of Intermediate 90a) (218 mg) in dichloromethane (10 ml) was added 2-(3,4-dichlorophenyl)acetyl chloride (243 mg) in dichloromethane (5 ml). The reaction mixture was stirred at RT for 4 days. The reaction mixture was diluted with CH₂CH₂, hydrolysed with 0.5M NaOH (aq.), stirred for 5 minutes and layers were separated. The organic layer was concentrated in vacuo affording the crude product as a brown oil. Purification by flash column chromatography (0.5% (7N NH₃ in MeOH)/CH₂Cl₂) yielded the product as a yellow oil.

### Example 90

### Salt

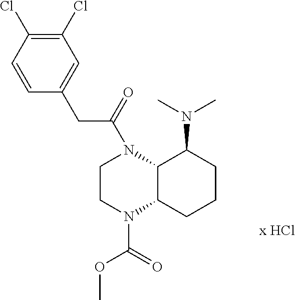 [SMILES: COC(=O)N1CCN(C(=O)Cc2ccc(Cl)c(Cl)c2)[C@@H]2[C@@H](N(C)C)CCC[C@@H]21]

Example 90 (free base) (85 mg) was dissolved in acetonitrile/water and lyophilized, yielding a white fluffy solid which was dissolved in CH₂Cl₂. Excess HCl in Et₂O (1 N) was added and the mixture was concentrated in vacuo to give the HCl-salt. The compound was resuspended in Et₂O, the solvent was decanted and the product was dried at 40° C. in a vacuum stove overnight to the product as HCl salt.

### Synthesis of Example 124

Intermediate 124a):

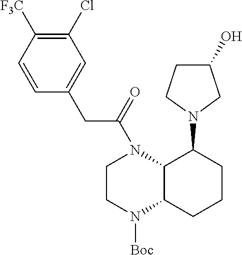 [SMILES: CC(C)(C)OC(=O)N1CCN(C(=O)Cc2ccc(C(F)(F)F)c(Cl)c2)[C@@H]2[C@@H](N3CC[C@H](O)C3)CCC[C@@H]21]

To a solution of 2-(3-chloro-4-(trifluoromethyl)phenyl)acetyl chloride (207 mg) in dichloromethane (2 ml), was added a solution of (4aR,5S,8aS)-tert-butyl 5-((S)-3-hydroxypyrrolidin-1-yl)octahydroquinoxaline-1(2H)-carboxylate (250 mg) in DCM (2 ml) at room temperature. The reaction mixture was stirred at RT for 3 h. The reaction mixture was diluted with dichloromethane (10 ml) and hydrolysed with 0.5 M NaOH (aq., 10 ml) to reach pH 12. The aqueous phase was separated and extracted twice with dichloromethane (2×10 ml). The combined organic phase was dried over Na₂SO₄, filtered and evaporated in vacuo. Purification by flash column chromatography (0.5-5.0% MeOH in CH₂Cl₂) yielded 217 mg product.

Intermediate 124b):

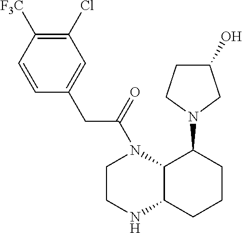 [SMILES: O=C(Cc1ccc(C(F)(F)F)c(Cl)c1)N1CCN[C@H]2CCC[C@H](N3CC[C@H](O)C3)[C@H]21]

To a solution of Intermediate 124a) (217 mg) in dichloromethane (1 ml), was added trifluoroacetic acid (0.5 ml) at room temperature. The reaction mixture was stirred at RT for 90 min. Trifluoroacetic acid (0.5 ml) was added and stirring at RT was continued for 16 hours. The reaction mixture was concentrated to dryness. The residue was dissolved in dichloromethane (10 ml) and washed with saturated NaHCO₃ (aq., 10 ml) and brine. The organic phase was dried over Na₂SO₄, filtered and the solvent was evaporated to yield 181 mg of product which was used in the next step without further purification.

### Example 124

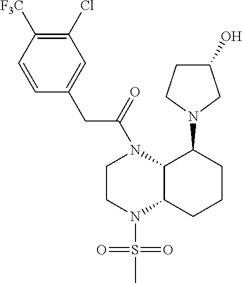 [SMILES: CS(=O)(=O)N1CCN(C(=O)Cc2ccc(C(F)(F)F)c(Cl)c2)[C@@H]2[C@@H](N3CC[C@H](O)C3)CCC[C@@H]21]

In a screw-cap vial, methanesulfonyl chloride (48.6 mg) was dissolved in dichloromethane (2 ml). At ambient temperature, Intermediate 124b) (180 mg) was added. The resulting mixture was stirred at RT for 45 min. Triethylamine (84 μl) was added and the reaction mixture was stirred at RT for another hour. An additional amount of methanesulfonyl chloride (29.6 mg) was added to the reaction mixture, which was stirred at RT for a further 30 min. The crude reaction mixture was concentrated to dryness. The residue was dissolved in CH₂Cl₂ (10 ml) and washed with NaOH (0.5 M, aq., 10 ml). The water phase was extracted twice with dichloromethane (2×10 ml). The combined organic phase was dried with sodium sulfate, filtered and the solvent was evaporated. Purification of the crude material was performed by flash column chromatography (0-5% MeOH in DCM) followed by purification by prep-LC to yield the product.

Biological Assays

A. κ Opioid Receptor Binding Assay (Rat Membrane Preparations)

The κ receptor affinities of the test items were determined in competition experiments with the radioligand [³H]U-69,593. Membrane homogenates prepared from guinea pig brains were used as receptor material. Non-specific binding was determined in the presence of a large excess of non-tritiated U-69,593 (10 μM) (see e.g. Siebert D. J. Pharmacol. 1994; 43:53-56, Naylor, A. J. Med. Chem. 1993; 36:2075-2083 and Kracht, D. Org. Biomol. Chem. 2010; 8: 212-225).

Data Analysis:

All experiments were carried out in triplicates using standard 96-well-multiplates (Diagonal). The IC₅₀-values were determined in competition experiments with six concentrations of the test compounds and were calculated with the program GraphPad Prism® 3.0 (GraphPad Software) by non-linear regression analysis. The Kᵢ-values were calculated according to Cheng and Prusoff (Cheng, Y.-C. Pharmacol. 1973; 22:3099-3108). The Kᵢ-values are given as mean values±SEM from three independent experiments.

B. κ Opioid Receptor Binding Assay (HEK-293 Cell Membrane Preparations)

Human opiate κ receptors expressed in HEK-293 cells are used in modified Tris-HCl buffer pH 7.4. A 30 μg aliquot is incubated with 0.6 nM [³H]Diprenorphine for 60 minutes at 25° C. Nonspecific binding is estimated in the presence of 10 μM naloxone. Membranes are filtered and washed, the filters are then counted to determine [³H]Diprenorphine specifically bound. Test compounds are screened at various concentrations (see e.g. Maguire, P. Eur. J. Pharmacol. 1992; 213:219-225).

### C. κ Opioid Receptor Functional Assay (GTPγS Binding)

Human recombinant opiate κ receptors stably expressed in HEK-293 cells are used. Test compound and/or vehicle is preincubated with the membranes (0.057 mg/ml) and 3 mM GDP in modified HEPES pH 7.4 buffer for 20 minutes at 25° C. and SPA beads are then added for another 60 minutes at 30° C. The reaction is initiated by 0.3 nM [³⁵S]GTPγS for an additional 30 minute incubation period. Test compound-induced increase of [³⁵S]GTPγS binding by 50 percent or more (>50%) relative to the 10 μM U-69593 response indicates possible opiate K receptor agonist activity. Compounds are screened at various concentrations.

TABLE 3
κ Opioid receptor binding and functional activity (determination as described in
biological assays A and C)

| | | | | % |
|---|---|---|---|---|
| | | | | activation |
| | | | | functional |
| | | | GTPγS | assay |
| Compound | structure | Kᵢ (nM) ± SEM | EC₅₀ (nM) | at 1 μM |
| Example 1 | 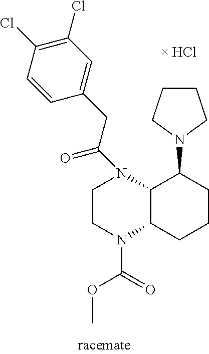 [SMILES: COC(=O)N1CCN(C(=O)Cc2ccc(Cl)c(Cl)c2)[C@@H]2[C@@H](N3CCCC3)CCC[C@@H]21] | 0.35 ± 0.06 | <10 | 109 |
| Example 89 | 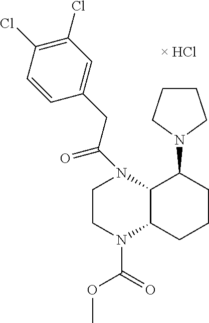 [SMILES: COC(=O)N1CCN(C(=O)Cc2ccc(Cl)c(Cl)c2)[C@@H]2[C@@H](N3CCCC3)CCC[C@@H]21] | 0.25 ± 0.08 | 1.99 | 107 |
| Reference compound A | 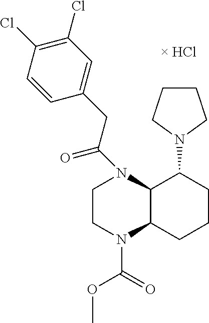 [SMILES: COC(=O)N1CCN(C(=O)Cc2ccc(Cl)c(Cl)c2)[C@H]2[C@H](N3CCCC3)CCC[C@H]21] | 96.1 ± 7.2 | 992 | 51 |
| Reference compound B | 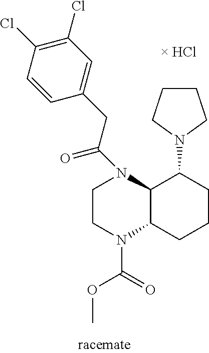 [SMILES: COC(=O)N1CCN(C(=O)Cc2ccc(Cl)c(Cl)c2)[C@H]2[C@H](N3CCCC3)CCC[C@@H]21] | 23.6 ± 6.4 | 110 | 90 |
| Reference compound C¹⁾ | 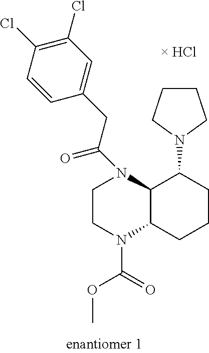 [SMILES: COC(=O)N1CCN(C(=O)Cc2ccc(Cl)c(Cl)c2)[C@H]2[C@H](N3CCCC3)CCC[C@@H]21] | 15.1 ± 2.3 | 149 | 85 |
| Reference compound D¹⁾ | 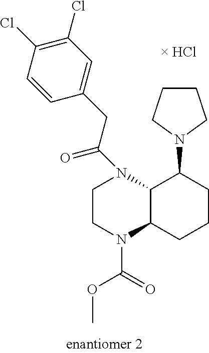 [SMILES: COC(=O)N1CCN(C(=O)Cc2ccc(Cl)c(Cl)c2)[C@H]2[C@H]1CCC[C@@H]2N1CCCC1] | 19.7 ± 7.2 | 125 | 92 |
| Reference compound E | 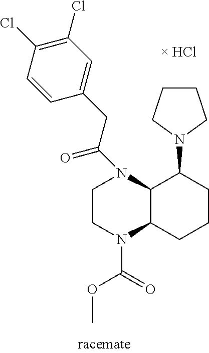 [SMILES: COC(=O)N1CCN(C(=O)Cc2ccc(Cl)c(Cl)c2)[C@@H]2[C@H]1CCC[C@@H]2N1CCCC1] | 57.9 ± 17 | 351 | 68 |
| Reference compound F²⁾ | 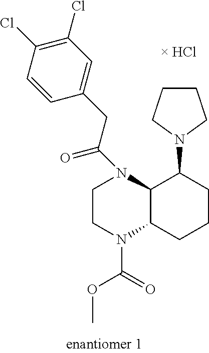 [SMILES: COC(=O)N1CCN(C(=O)Cc2ccc(Cl)c(Cl)c2)[C@H]2[C@@H](N3CCCC3)CCC[C@@H]21] | 115 ± 38 | n.d. | n.d. |
| Reference compound G²⁾ | 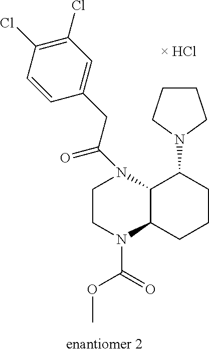 [SMILES: COC(=O)N1CCN(C(=O)Cc2ccc(Cl)c(Cl)c2)[C@@H]2[C@H](N3CCCC3)CCC[C@H]21] | 19.8 ± 6.0 | n.d. | n.d. |

¹⁾Enantiomeric structures of Reference compounds C and D are assigned arbitrarily.
²⁾Enantiomeric structures of Reference compounds F and G are assigned arbitrarily.

TABLE 4
κ Opioid receptor functional activity for reference compounds from
WO2009/080745 (determination as described in biological assay C)

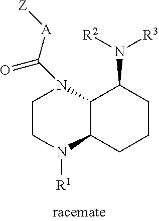

| | | | | | activation |
|---|---|---|---|---|---|
| | | | | EC₅₀ (nM) | (% of |
| Reference | | | | [³⁵S]GTPγS | control) |
| compound | R¹ | NR²R³ | A—Z | binding | at 1 μM |
| H | 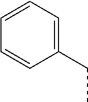 | 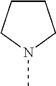 | 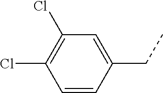 | 29 | 97 |
| I | 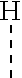 | 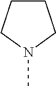 | 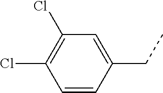 | 33 | 114 |
| J | 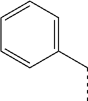 | 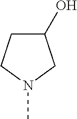 | 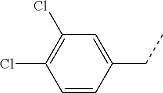 | 64 | 77 |
| K | 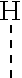 | 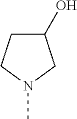 | 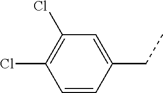 | 35 | 108 |
| L | 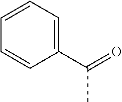 | 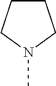 | 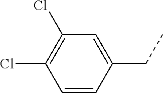 | 260 | 74 |
| M | 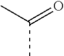 | 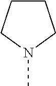 | 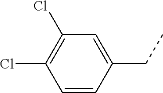 | 330 | 78 |
| N | 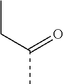 | 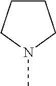 | 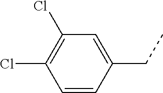 | 600 | 63 |
| B | 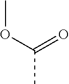 | 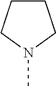 | 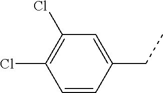 | 110 | 90 |
| O | 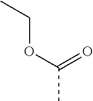 | 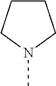 | 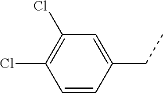 | 150 | 88 |
| P | 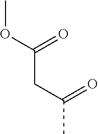 | 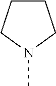 | 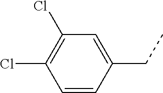 | 220 | 81 |
| Q | 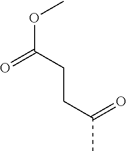 | 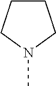 | 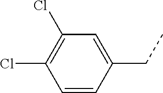 | 530 | 61 |
| R | 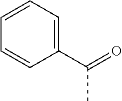 | 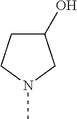 | 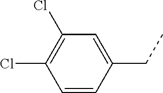 | 470 | 63 |
| S | 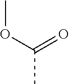 | 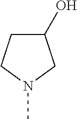 | 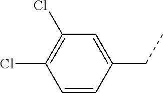 | 85 | 91 |
| T | 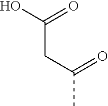 | 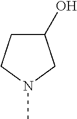 | 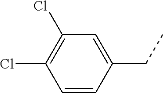 | >1,000 | 11 |
| U | 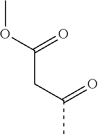 | 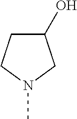 | 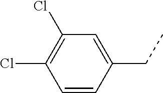 | 170 | 86 |
| V | 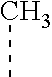 | 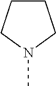 | 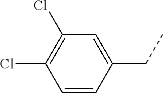 | 20 | 107 |
| W | 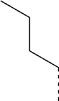 | 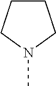 | 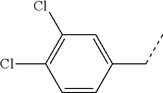 | 12 | 113 |
| X | 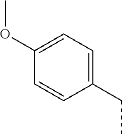 | 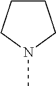 | 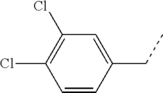 | 26 | 91 |
| Y | 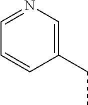 | 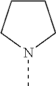 | 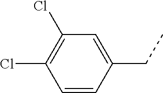 | 34 | 96 |
| Z | 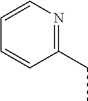 | 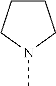 | 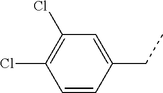 | 41 | 103 |
| AA | 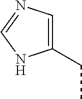 | 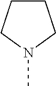 | 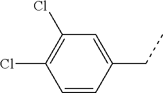 | 56 | 89 |
| AB | 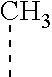 | 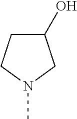 | 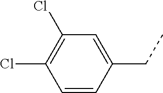 | 79 | 99 |
| AC | 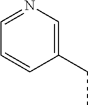 | 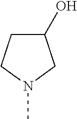 | 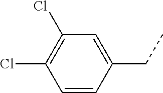 | 77 | 102 |
| AD | 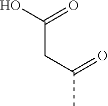 | 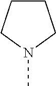 | 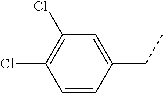 | >1,000 | 22 |
| AE | 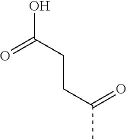 | 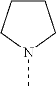 | 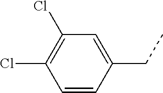 | >1,000 | 43 |

TABLE 5
κ Opioid receptor functional activity for selected examples (determination as
described in biological assay C)

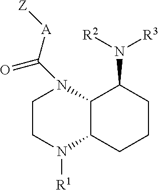

| | | | | | activation |
|---|---|---|---|---|---|
| | | | | EC₅₀ (nM) | (% of |
| | | | | [³⁵S]GTPγS | control) |
| Example | R¹ | NR²R³ | A—Z | binding | at 1 μM |
| 15 | 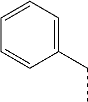 | 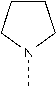 | 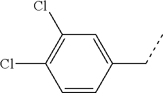 | 7.39 | 89 |
| 39 | 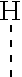 | 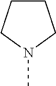 | 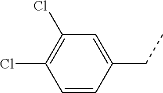 | 1.64 | 102 |
| 31 | 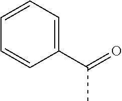 | 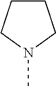 | 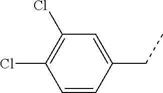 | 2.11 | 93 |
| 33 | 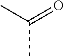 | 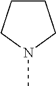 | 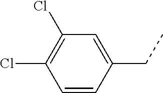 | 3.96 | 101 |
| 25 | 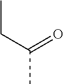 | 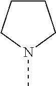 | 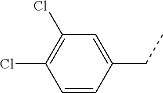 | 2.55 | 102 |
| 1 | 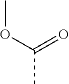 | 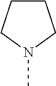 | 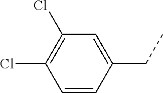 | 2.2 | 109 |
| 36 | 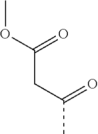 | 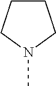 | 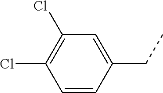 | 9.74 | 93 |
| 11 | 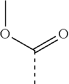 | 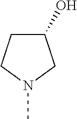 | 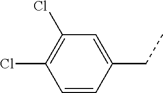 | 11.7 | 92 |
| 53 | 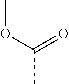 | 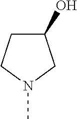 | 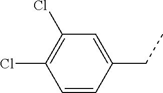 | 6.1 | 97 |
| 52 | 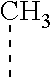 | 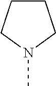 | 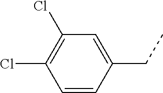 | 2.46 | 107 |
| 48 | 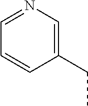 | 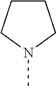 | 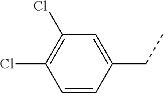 | 4.99 | 100 |
| 49 | 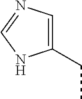 | 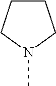 | 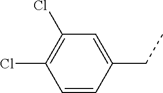 | 6.65 | 112 |
| 37 | 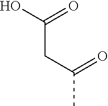 | 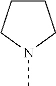 | 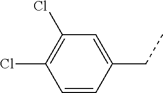 | 22 | 87 |

The data in Table 4 show that all but three reference compounds from WO2009/080745 are functional agonists of the kappa opioid receptor exhibiting EC₅₀ values below 1 μM. Compounds bearing a carboxylate function (T, AD and AE) show no or little activation of the kappa opioid receptors at 1 μM. By direct comparison of reference compounds from Table 4 with examples from the present invention having the same decoration of the core structure as shown in Table 5 it can clearly be seen that all newly synthesized compounds have lower EC_{50S} in the kappa receptor GTPγS binding assay. Examples 15, 48, 52 and 49 show 4- to 8 fold lower EC_{50S} compared to the analogs from WO2009/080745. For all other analogs the difference is even higher (14fold to 235fold). Example 37 activates the kappa opioid receptor with an EC₅₀ of 22 nM, whereas its counterpart reference compound AD exhibits an EC₅₀>1 μM. There is not a single compound according to the present invention with a higher EC₅₀ compared to WO2009/080745. Thus, the compounds according to formula (1) of the present invention (having a 4aR,5S,8aS stereochemistry) provide for improved and unexpected technical effects.

TABLE 6
κ Opioid receptor binding (determination
as described in biological assays B)

| | % binding | % binding | % binding |
|---|---|---|---|
| Example | at 10 nM | at 100 nM | at 1 μM |
| 2 | | 41 | |
| 3 | | 68 | |
| 4 | | 43 | |
| 5 | | 73 | |
| 6 | | 68 | |
| 7 | | 58 | |
| 8 | | 64 | |
| 9 | | 43 | |
| 10 | | 51 | |
| 11 | 56 | 83 | |
| 12 | | 72 | |
| 13 | 66 | 92 | |
| 14 | | 73 | 88 |
| 15 | | | 92 |
| 16 | | 95 | 100 |
| 17 | | 92 | 97 |
| 18 | | | 94 |
| 19 | 47 | | 97 |
| 20 | 57 | | 98 |
| 21 | | | 86 |
| 22 | | | 68 |
| 23 | 43 | | 99 |
| 24 | 47 | | 99 |
| 25 | | | 93 |
| 26 | 42 | | 97 |
| 27 | 58 | | 99 |
| 28 | 55 | | 99 |
| 29 | | | 96 |
| 30 | 53 | | 98 |
| 31 | 52 | | 96 |
| 32 | 42 | | 92 |
| 33 | | | 93 |
| 34 | | 51 | |
| 35 | | 87 | |
| 36 | | 70 | |
| 37 | | 49 | |
| 38 | | 53 | |
| 39 | | 89 | |
| 40 | | 85 | |
| 41 | | 92 | |
| 42 | | 81 | |
| 43 | | 82 | |
| 44 | | 41 | |
| 45 | | 89 | |
| 46 | | 76 | |
| 47 | | 72 | |
| 48 | | 72 | |
| 49 | | 69 | |
| 50 | | 98 | |
| 51 | | 95 | |
| 52 | | 83 | |
| 53 | | 70 | |
| 54 | | 59 | |
| 55 | | 53 | |
| 56 | | 78 | |
| 57 | | 56 | |
| 58 | | 62 | |
| 59 | | 75 | |
| 60 | | 63 | |
| 61 | | 77 | |
| 62 | | 70 | |
| 63 | | 81 | |
| 64 | | 72 | |
| 65 | | 92 | |
| 66 | | 65 | |
| 67 | | 96 | |
| 68 | | 72 | |
| 69 | | 52 | |
| 70 | | 85 | |
| 84 | | 89 | |
| 85 | | 64 | |
| 86 | | 95 | |
| 87 | | 66 | |
| 89 | | 61 | |
| 107 | | 95 | |
| 111 | | 105 | |
| 123 | | 68 | |
| 124 | | 88 | |
| 125 | | 80 | |
| 126 | | 82 | |
| 127 | | 66 | |
| 128 | | 92 | |
| 129 | | 80 | |
| 130 | | 70 | |
| 131 | | 78 | |
| 132 | | 71 | |
| 133 | | 74 | |
| 134 | | 67 | |
| 135 | | 76 | |
| 136 | | 96 | |
| 137 | | 71 | |
| 138 | | 66 | |
| 139 | | 66 | |
| 140 | | 59 | |
| 141 | | 91 | |
| 142 | | 58 | |
| 143 | | 66 | |
| 144 | | 91 | |
| 145 | | 62 | |
| 146 | | 76 | |
| 147 | | 88 | |
| 148 | | 92 | |
| 149 | | 81 | |
| 150 | | 97 | |

TABLE 7
κ Opioid functional activity (determination as described
in biological assays C) EC₅₀ values are grouped in three classes:
a ≦ 10 nM; b > 10 nM and ≦100 nM; c > 100 nM and ≦1 μM

| | GTPγS | % activation functional |
|---|---|---|
| Example | EC₅₀ (nM) | assay at 1 μM |
| 1 | a | 109 |
| 3 | b | 105 |
| 5 | a | 105 |
| 6 | b | 97 |
| 7 | b | 101 |
| 8 | b | 97 |
| 10 | b | 100 |
| 11 | b | 92 |
| 12 | b | 96 |
| 13 | a | 98 |
| 14 | a | 104 |
| 15 | a | 89 |
| 16 | a | 102 |
| 17 | a | 92 |
| 18 | a | 112 |
| 19 | a | 100 |
| 20 | a | 96 |
| 21 | b | 103 |
| 22 | c | 78 |
| 23 | a | 91 |
| 24 | a | 105 |
| 25 | a | 102 |
| 26 | a | 119 |
| 27 | a | 103 |
| 28 | a | 111 |
| 29 | a | 96 |
| 30 | a | 110 |
| 31 | a | 93 |
| 32 | a | 88 |
| 33 | a | 101 |
| 34 | b | 107 |
| 35 | a | 108 |
| 36 | a | 93 |
| 37 | b | 87 |
| 38 | b | 109 |
| 39 | a | 102 |
| 40 | a | 96 |
| 41 | a | 81 |
| 42 | a | 96 |
| 43 | a | 107 |
| 45 | a | 91 |
| 46 | a | 96 |
| 47 | a | 101 |
| 48 | a | 100 |
| 49 | a | 112 |
| 50 | a | 118 |
| 51 | a | 97 |
| 52 | a | 107 |
| 53 | a | 97 |
| 54 | b | 104 |
| 55 | a | 101 |
| 56 | a | 102 |
| 57 | b | 112 |
| 58 | b | 96 |
| 59 | b | 109 |
| 60 | b | 105 |
| 61 | b | 108 |
| 62 | b | 104 |
| 63 | b | 106 |
| 64 | b | 102 |
| 65 | a | 104 |
| 66 | b | 100 |
| 67 | a | 106 |
| 68 | b | 104 |
| 69 | b | 101 |
| 70 | a | 96 |
| 71 | b | 113 |
| 72 | a | 106 |
| 73 | b | 109 |
| 74 | c | 88 |
| 75 | a | 113 |
| 76 | b | 106 |
| 77 | b | 98 |
| 78 | c | 64 |
| 79 | b | 102 |
| 80 | c | 76 |
| 81 | b | 102 |
| 82 | b | 97 |
| 83 | a | 111 |
| 84 | b | 99 |
| 85 | b | 101 |
| 86 | b | 86 |
| 87 | b | 109 |
| 88 | a | 107 |
| 89 | b | 100 |
| 90 | a | 96 |
| 91 | a | 106 |
| 92 | b | 105 |
| 93 | a | 101 |
| 94 | b | 103 |
| 95 | a | 104 |
| 96 | a | 109 |
| 97 | a | 113 |
| 98 | a | 105 |
| 99 | a | 100 |
| 100 | a | 105 |
| 101 | a | 99 |
| 102 | a | 106 |
| 103 | a | 98 |
| 104 | a | 111 |
| 105 | a | 116 |
| 106 | a | 92 |
| 107 | a | 104 |
| 108 | a | 100 |
| 109 | a | 110 |
| 110 | b | 92 |
| 111 | a | 105 |
| 112 | a | 93 |
| 113 | a | 102 |
| 114 | b | 93 |
| 115 | b | 103 |
| 116 | b | 111 |
| 118 | a | 104 |
| 119 | b | 102 |
| 120 | a | 100 |
| 121 | a | 106 |
| 122 | a | 105 |
| 123 | a | 99 |
| 124 | a | 97 |
| 125 | a | 99 |
| 126 | a | 94 |
| 127 | b | 100 |
| 128 | a | 95 |
| 129 | a | 104 |
| 130 | b | 102 |
| 131 | a | 104 |
| 132 | a | 106 |
| 133 | a | 105 |
| 134 | a | 102 |
| 135 | a | 105 |
| 136 | a | 108 |
| 137 | a | 101 |
| 138 | b | 108 |
| 139 | a | 104 |
| 140 | b | 103 |
| 141 | a | 99 |
| 142 | b | 106 |
| 143 | b | 102 |
| 144 | a | 106 |
| 145 | b | 103 |
| 146 | a | 103 |
| 147 | a | 105 |
| 148 | a | 93 |
| 149 | a | 97 |
| 150 | a | 92 |

D. In Vivo Model for Pruritus Associated with the Oxazolone Model of a Delayed Type Hypersensitivity Reaction

Scratching activity in mice is measured after topical application of the test compound. Ear thickness is measured and histology parameters are determined (see e.g. Elliott G. R. An automated method for registering and quantifying scratching activity in mice: use for drug evaluation. J. Pharmacol. Toxicol. Methods. 2000; 44:453-459 and Gijbels M. J. Therapeutic interventions in mice with chronic proliferative dermatitis (cpdm/cpdm). Exp. Dermatol. 2000; 9:351-358).

Treatment with example 89 resulted in an accelerated decrease of ear thickness as compared to vehicle treated animals. The number of scratch events was significantly reduced. The anti-inflammatory properties of example 89 were confirmed histologically. Treatment with example 89 resulted in a reduction of epidermal thickness, inflammatory infiltrate and epidermal oedema (semi-quantitative analysis).

E. In Vivo Model of Chronic Oxazolone-Induced Ear Inflammation

Mice are challenged several times with oxazolone following an initial sensitization. Ear thickness is measured daily during the treatment period with topical application of the test compound (see e.g. Ottosen E. R. J. Med. Chem. 2003; 46: 5651-5662). At the end of the study ear weight is determined Ears are characterized histologically and by immunofluorescence. Gene expression was quantified (RT-qPCR).

Treatment with example 89 resulted in a dose dependent decreased ear thickness as compared to vehicle control. The anti-inflammatory properties of example 89 were confirmed histologically. Treatment with example 89 resulted in a reduction of epidermal thickness, inflammatory infiltrate and epidermal oedema (semi-quantitative analysis).

Similar results were obtained when mice were treated with examples 112, 118, 122, 125 or 145.

mRNA expression of proinflammatory cytokines IL-6 and TNF-α, of markers of the inflammatory infiltrate for mast cells (CD117, FcεRI) and necrophiles (myeloperoxidase) and of adhesion molecules (CD26E, ICAM-1) was down-regulated in mice treated with example 89 Immunohistochemistry showed a dose dependent reduction of the inflammatory infiltrate (CD117⁺ mast cells and Gr-1⁺ neutrophils).

F. Mouse Model of Topical Arachidonic Acid-Induced Ear Inflammation

Arachidonic acid in acetone is applied topically to the anterior and posterior surfaces of the right ear of mice. Test substances are similarly applied 30 minutes before and 15 minutes after arachidonic acid. Ear swelling is measured 1 h after application of arachidonic acid. Scratching activity is monitored for 1 h following the application of arachidonic acid. Ear weight and histology parameters are determined at the end of the study (see e.g. Chang J. Eur. J. Pharmacol. 1987; 142:197-205).

Treatment with example 89 (topical and s.c.) prevented the increase in ear thickness observed for the vehicle control. Scratching activity was significantly reduced. Both effects are dose dependent.

Similar results were obtained when mice were treated with example 97.

Treatment with examples 81, 112,114, 118, 122, 125 and 145, respectively, (topical) dose dependently prevented the increase in ear thickness observed for the vehicle control.

G. Acetic Acid-Induced Writhing Assay in Mice

Analgesic activity against visceral or chemical pain is assessed by application of the test compound prior to application of an i.p. injection of acetic acid. The number of writhing responses that occur in response to acetic acid are counted (see e.g. Barber A. Med. Res. Rev. 1992; 12:525-62 and Ramabadran K. Pharm. Res. 1986,3:263-270).

Treatment with example 89 (s.c.) resulted in a significant, dose dependent reduction in the number of writhing responses. Similar effects were observed for examples 96 and 97.

H. UVB-Induced Inflammatory Pain In Rats

Male, Sprague-Dawley rats receive a single exposure of UVB radiation to the left hind paw. Mechanical hyperalgesia is assessed using a digital Randall-Selitto device (dRS). Thermal hyperalgesia is measured using a plantar test apparatus (see e.g. Davies S. L. J. Neurosci. Methods 2005; 148:161-166, Bishop T. Pain 2007; 131:70-82 and Graham I. J. Invest. Dermatol. 2004; 122:183-189).

Treatment with example 89 (s.c.) resulted in a significant, dose dependent reduction of thermal hyperalgesia.

I. Vasculitis Model in Mice

C57BL/6 mice receive an intradermal injection of LPS. On the following day vasculitis is induced by intradermal injection of TNF-α. In addition Evan's blue is injected. 24 hours following the injection of TNF-α mice are scarified. Ear thickness is measured and the degree of vasculitis is assessed by counting petechiae. The content of Evan's blue in the ear tissue is a marker for vascular permeability. Ears are analyzed by histology, FACS and RT-qPCR.

Treatment with example 89 resulted in a reduction of ear thickness and a reduced number of petechiae. In histology a reduced inflammatory infiltrate was seen. The observed effects were dose dependent.

J. Imiquimod-Induced Psoriasis in Mice

Psoriasis in Balb/c mice is induced by daily application of topical Imiquimod for 8 days. Animal are treated with the test items (topical or systemically). Scratching was monitored. On day 9 the skin phenotype is characterized. Skin is analyzed histologically. Lymph nodes are analyzed by flow cytometry and RT-qPCR.

Treatment with example 89 (s.c. or i.v., resp.) resulted in a decreased size of the rete ridges as compared to vehicle control. Furthermore, scratch numbers were lower in treated mice.

K. DSS-Induced Colitis in Mice

Colitis is induced by treatment of C57BL/6 mice with 2.5% dextran sulfate (DSS) in the drinking water for 7 days. Mice are treated with the test item. Weight is monitored daily. At day 8 mice are scarified. A haemocult test is performed. The size of the colon is measured. Colitis is determined using a scoring system in H&E stains.

Treatment with example 89 resulted in a decreased weight loss compared to vehicle control. The reduction in colon sized induced by DSS was normalized in treated mice.

L. Effects on Chloroquine-Induced Scratching

Compounds are intrathecally injected in a volume of 5 μl, 10 min before the i.d. injection of chloroquine (100 μg/10 μl) in the rostral back. Following the i.d. cheek injection, mice are placed in an arena with a clear glass floor and videotaped from beneath for 30 min. Videotapes are reviewed by blinded investigators, who count the number of hindlimb scratch bouts.

Treatment with examples 81 and 114 significantly inhibited chloroquine-evoked scratching.

M. Pharmacokinetic Studies, Evaluation of Clinical Signs

The test items are administered intravenously to Wistar rats. Blood samples are taken after 15 minutes and after 1 h following administration. Perfused brains are collected 1 h following administration of the test item. Brain and plasma concentrations are measured. Clinical signs are monitored 15 minutes and 1 h after dosing.

N. hERG Inhibition Assay

The effect of the test items on the hERG tail current in stably transected HEK-293 cells is assessed (see e.g. Zhou Z. Biophys. J. 1998; 74:230-241).

Examples of Pharmaceutical Compositions

### Composition Example 89

Cream

| | |
|---|---|
| Compound 89 | 1.00 |
| Cetostearyl alcohol | 7.00 |
| Macrogol-6-cetostearyl ether | 1.50 |
| Macrogol-25-cetostearyl ether | 1.50 |
| Liquid paraffin | 12.00 |
| Propylene glycol | 8.00 |
| Methylparaben | 0.15 |
| Ethylparaben | 0.08 |
| Butylhydroxytoluene | 0.04 |
| Disodium edetate | 0.05 |
| Water | 68.68 |

### Composition Example 97

Gel

| | |
|---|---|
| Compound 97 | 0.50 |
| Ethanol | 15.00 |
| Polyoxyl 40 Hydrogenated Castor Oil | 1.00 |
| Butylhydroxytoluene | 0.04 |
| Disodium edetate | 0.05 |
| Carbomer | 0.50 |
| Triethanolamine | 0.70 |
| Water | 82.21 |

### Composition Example 107

As a specific embodiment of an oral composition of a compound of the present invention, 19 mg of Example 107 is formulated with sufficient finely divided lactose to provide a total amount of 580 to 590 mg to fill a size 0 hard gelatine capsule.

### Composition Example 119

As another specific embodiment of an oral composition of a compound of the present invention, 23 mg of Example 119 is formulated with sufficient finely divided lactose to provide a total amount of 580 to 590 mg to fill a size 0 hard gelatine capsule.

1. A perhydroquinoxaline compound according to the general formula (1) as shown below or a solvate or hydrate thereof or a pharmaceutically acceptable salt thereof: 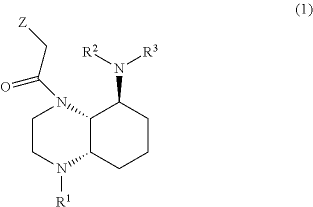
wherein:
R¹ is chosen from the group comprising H; C₃-C₁₈-cycloalkyl; (COO(C₁-C₁₀-alkyl);
phenylalkyl with C₁-C₆-alkyl, wherein the phenyl radical can be substituted by one or more identical or different groups chosen from the group comprising halogen, C₁-C₆-alkyloxy, NH₂, NH(C₁-C₅-alkyl), N(C₁-C₅-alkyl)₂, OH, SO₂(C₁-C₅-alkyl), SO(C₁-C₃-alkyl), CF₃, CN, NO₂, SO₂N(C₁-C₅-alkyl₂, SO₂NH₂, SO₂NH(C₁-C₅-alkyl), SO₂NH(aryl), SO₂NH(phenyl) and/or SO₂NH(heteroaryl);
C₁-C₁₀-acyl; heterocyclylacyl containing one, two, three or four hetero atoms chosen from the group comprising NH, O and/or S; phenylacyl, wherein the acyl radical is a C₁-C₆-acyl radical and the phenyl radical can be substituted by one or more identical or different groups chosen from the group comprising halogen, C₁-C₆-alkyloxy, COO(C₁-C₆-alkyl), NH₂, NH(C₁-C₅-alkyl), N(C₁-C₅-alkyl)₂, CONH₂, CONH(C₁-C₆-alkyl), CON(C₁-C₆-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl);
mono-, bi- or tricyclic heteroaryl containing one, two, three or four hetero atoms chosen from the group comprising N, O and/or S;
mono-, bi- or tricyclic heteroarylalkyl containing one, two, three or four hetero atoms Chosen from the group comprising N, O and/or S, wherein the alkyl radical is a C₁-C₆ alkyl radical;
mono-, bi- or bicyclic heteroaryl/acyl containing one, two, three or four hetero atoms chosen from the group comprising N, O and/or S, wherein the acyl radical is a C₁-C₆-acyl radical and the heteroaryl radical can be substituted by one or more identical or different groups chosen from the group comprising halogen, C₁-C₆-alkyloxy, COO(C₁-C₆-alkyl), NH₂, NH(C₁-C₅-alkyl), N(C₁-C₅-alkyl)₂, CONH₂, CONH(C₁-C₆-alkyl), CON(C₁-C₆-alkyl)₂, OH, CF₃, CN, NO₂, and/or SO₂NH₂;
mono-, bi- or tricyclic (heteroaryl)alkenylacyl containing one, two, three or four hetero atoms chosen from the group comprising N, O and/or S, wherein the acyl radical is a C₁-C₆-acyl radical and the alkenyl radical is a C₂-C₆-alkenyl radical;
C(O)NH(C₁-C₁₀-alkyl); C(O)N(C₁-C₁₀-alkyl)₂, wherein the two alkyl radicals may form a saturated substituted or unsubstituted ring with the N atom; C(O)NH(aryl); C(O)NH(benzyl); C(O)(C₃-C₁₀-cycloalkyl); COO(aryl); COO(benzyl); COO(C₃-C₁₀-cycloalkyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0, 1, 2, 3 or 4; C(O)NH—(CH₂)ₖ—COO(C₁-C₆-alkyl), wherein k is 0, 1, 2, 3 or 4; C(O)NH—(CH₂)ₗ—CONH₂, wherein l is 0, 1, 2, 3 or 4;
COO—(CH₂)ₘ—COOH, wherein m is 0, 1, 2, 3 or 4; COO—(CH₂)ₙ—COO(C₁-C₁₀-alkyl), wherein n is 0, 1, 2, 3 or 4; COO—(CH₂)ₚ—C(O)NH₂, wherein p is 0, 1, 2, 3 or 4; C(O)—(CH₂)_{q}—COOH, wherein q is 0, 1, 2, 3 or 4; C(O)—(CH₂)ᵣ—COO(C₁-C₁₀-alkyl), wherein r is 0, 1, 2, 3 or 4; C(O)—(CH₂)ₛ—C(O)NH₂, wherein s is 0, 1, 2, 3 or 4; C(O)—(CH₂)ₜ—C(O)NH(C₁-C₆-alkyl), wherein t is 0, 1, 2, 3 or 4; C(O)—(CH₂)ᵤ—C(O)N(C₁-C₆-alkyl)₂, wherein u is 0, 1, 2, 3 or 4;
C(O)—(CH₂)ᵥ—NH₂, wherein v is 0, 1, 2, 3 or 4; C(O)—-(CH₂)_{w}—OR′, wherein w is 0, 1, 2, 3 or 4 and R′ is H or C₁-C₆-acyl; C(O)—(CH₂)ₓ—C(O)NH—(CH₂)_{y}C(O)NH₂, wherein x is 0, 1, 2 or 3 and wherein y is 0, 1, 2 or 3;
SO₂(C₁-C₆-alkyl); SO₂—(CH₂)_{z}-heteroaryl, wherein z is 0, 1, 2 or 3; SO₂(CH₂)ₐ-heterocyclyl, wherein a is 0, 1, 2 or 3 and wherein the heterocyclyl residue may be substituted by one or more identical or different substituents chosen from the group comprising halogen, OH, CN, oxo and/or C₁-C₆-alkoxy; SO₂ or SO₂NH(C₁-C₆-alkyl), wherein the alkyl radical can be substituted by halogen, C₁-C₄-alkoxy and/or OH; SO₂NH(C₃-C₆-cycloalkyl); SO₂NH—C(O)O(C₁-C₆-alkyl);
R², IV are in each case identical or independent of each other and are chosen from the group comprising H; C₃-C₁₀-cycloalkyl,
or
R² and R³ form, together with the nitrogen to which they are bonded, a saturated or unsaturated 3- to 8-membered N-heterocycle, wherein this can be substituted by one or more identical or different groups chosen from the group comprising halogen, OH, C₁-C₄-alkyloxy, COOH, COO(C₁-C₁₀-alkyl), CONH₂, CONH(C₁-C₁₀-alkyl), CON(C₁-C₁₀-alkyl)₂, CN, and/or O—C(O)(C₁-C₆ alkyl);
Z is chosen from the group comprising phenyl, which can be substituted by one or more identical or different groups chosen from the group comprising halogen, C₁-C₅-alkyl, C₁-C₅-alkoxy, NH₂, NH(C₁-C₅-alkyl), N(C₁-C₅-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl), wherein the substituents may form a ring;
a mono- or bicyclic aryl or heteroaryl containing one or two hetero atoms Chosen from the group comprising N, O and/or S, wherein the aryl or heteroaryl group can be substituted by one or more identical or different groups chosen from the group comprising halogen, C₁-C₄-alkoxy, NH₂, NH(C₁-C₅-alkyl), N(C₁-C₅-alkyl)₂, OH, SO₂(C₁-C₅-alkyl), SO(C₁-C₅-alkyl), CF₃, CN, NO₂, SO₂N(C₁-C₅-alkyl)₂, SO₂NH₂, SO₂NH(C₁-C₅-alkyl), SO₂NH(aryl), SO₂NH(phenyl) and/or SO₂NH(heteroaryl).

2. The compound according to claim 1, wherein in general formula (1):
R¹ is chosen from the group comprising H; C₁-C₃-alkyl; COO(C₁-C₄-alkyl); benzyl; C₁-C₄-acyl; C(O)C₄-C₆-cycloalkyl; heterocyclylacyl containing NH or O in the ring; phenylacyl, wherein the aryl radical is a C acyl radical and the phenyl radical can be substituted by one or more identical or different groups chosen from the group comprising COO(C₁-C₃-alkyl) and CONH₂;
mono-cyclic heteroaryl containing one hetero atom chosen from the group of N, O and S;
mono-cyclic beteroarylalkyl containing one or two hetero atom chosen from the group of N, O and S, wherein the alkyl radical is a C₁-C₃ alkyl radical;
mono-cyclic heteroarylacyl containing one or two hetero atoms chosen from the group of N, O and S, wherein the acyl radical is a C₁-acyl radical and the heteroaryl radical can be substituted by one or more identical or different groups chosen from the group comprising COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals may form a saturated halogen substituted or unsubstituted ring with the N atom; C(O)NH(phenyl); C(O)NH(benzyl); C(O)(C₃-C₆-cycloalkyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ₗ—CONH₂, wherein l is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein in is 0 or 1; COO—(CH₂)ₖ—COO(C₁-C₃-alkyl), wherein n is 0 or 1; COO—(CH₂)ᵣ—C(O)NH₂, wherein p is 0 or 1; C(O)—(CH₂)_{q}—COOH, wherein q is 0 or 1; C(O)—(CH₂)ᵣ—COO(C₁-C₃-alkyl), wherein r is 0 or 1; C(O)—(CH₂)ₛ—C(O)NH₂, wherein s is 0 or 1; C(O)—(CH₂)ₜ—C(O)NH(C₁-C₃-alkyl), wherein t is 0 or 1; C(O)—(CH₂)ᵤ—C(O)N(C₁-C₃-alkyl)₂, wherein u is 0 or 1;
(C)—(O)—(CH₂)ᵥ—NH₂, wherein v is 0 or 1; C(O)—(CH₂)_{w}—OR′, wherein w is 0 or 1 and R′ is H or acetyl; C(O)—(CH₂)ₓ—C(O)NH—(CH₂)_{y}C(O)NH₂, wherein x is 0 or 1 and wherein y is 0 or 1;
SO₂(C₁-C₆-alkyl); SO₂—(CH₂)-heteroaryl, wherein z is 0 or 1; SO₂(CH₂)ₐ-heterocyclyl, wherein a is 0 or 1, wherein the heteroatoms are O, N, and/or S and wherein the heterocyclyl residue may be substituted by one or more identical or different substituents Chosen from the group comprising F, Cl, OH, CN, oxo and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);
R², R³ are identical or different and are chosen from the group comprising H, methyl, ethyl, propyl, and i-propyl,
or
R² and R³ form, together with the nitrogen to which they are bonded, a saturated or mono-unsaturated 4 to 6-membered N-heterocycle, wherein this can be substituted by one or more identical or different groups chosen from the group comprising F, Cl, OH, CONH₂, CN, and/or O—C(O)(C₁-C₃ alkyl);
Z is chosen from the group comprising
phenyl, which can be substituted by one or more identical or different groups chosen from the group comprising F, Cl, C₁-C₃-alkoxy, OH, CF₃, and NO₂, wherein two O substituents may be connected by an ether bridge to form a ring or wherein two C₁-C₃-alkyl groups may be connected to form a saturated ring; and
a mono- or bicyclic aryl or heteroaryl containing one hetero atom chosen from the group of N and S, wherein the aryl or heteroaryl group can be substituted by one or more identical or different groups chosen from the group comprising F, Cl, C₁-C₃-alkyl, C₁-C₃-alkoxy, OH, CF₃, and NO₂.

3. The compound according to claim 1, wherein in general formula (1):
R¹ is chosen from the group consisting of
heterocyclylacyl containing NH or O in the ring; phenylacyl, wherein the acyl radical is a C₁-acyl radical and the phenyl radical is substituted by one or more of COO(C₁-C₃-alkyl) and CONH₂;
mono-cyclic heteroarylacyl containing one or two hetero atoms chosen from the group of N, O and S, wherein the acyl radical is a C₁-aryl radical and the heteroaryl radical is substituted by one or more of COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals form a saturated halogen substituted or unsubstituted ring with the N atom; C(O)NH(phenyl); C(O)NH(benzyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)ₛ—C(O)NH₂wherein s is 0 or 1; C(O)—(CH₂)ₜ—C(O)NH(C₁-C₃-alkyl), wherein t is 0 or 1; C(O)—(CH₂)ᵤ—C(O)N(C₁-C₃-alkyl)₂, wherein u is 0 or 1;
C(O)—(CH₂)ᵥ—NH₂, wherein v is 1; C(O)—(CH₂)_{w}—OR′, wherein w is 1 and R′ is H or acetyl;
SO₂(C₁-C₆-alkyl), SO₂—(CH₂)₂-hetero aryl, wherein Z is 0 or 1; SO₂(CH₂)ₐ-heterocyclyl, wherein a is 0 or 1, wherein the heteroatoms are O, N, and/or S and wherein the heterocyclyl residue may be substituted by one or more identical or different substituents chosen from the group comprising F, Cl, OH, CN, two and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);
R², R³ are identical or different and are chosen from the group comprising H, methyl, ethyl, n-propyl, and i-propyl,
or
R² and R³ form, together with the nitrogen to which they are bonded, a saturated or mono-unsaturated 4 to 6-membered N-heterocycle, wherein this can be substituted by one or more identical or different groups chosen from the group comprising F, Cl, OH, CONH₂, CN, and/or O—C(O)(C₁-C₃ alkyl);
Z is chosen from the group comprising
phenyl, which can be substituted by one or more identical or different groups chosen from the group comprising F, Cl, C₁-C₃-alkyl, C₁-C₃-alkoxy, OH, CF₃, and NO₂, wherein two OH substituents may be connected by an ether bridge to form a ring or wherein two C₁-C₃-alkyl groups may be connected to form a saturated ring; and
a mono- or bicyclic aryl or heteroaryl containing one hetero atom chosen from the group of N and S, wherein the aryl or heteroaryl group can be substituted by one or more identical or different groups chosen from the group comprising F, Cl, C₁-C₃-alkyl, C₁-C₃-alkoxy, OH, CF₃, and NO₂.

4. The compound according to claim 1, wherein in general formula (1):
R¹ is chosen from the group comprising H; C₁-C₃-alkyl; COO(C₁-C₄-alkyl);
benzyl;
C₁-C₄-acyl; C(O)C₄-C₆-cycloalkyl; heterocyclylacyl containing NH or O in the ring; phenylacyl, wherein the acyl radical is a C₁-acyl radical and the phenyl radical can be substituted by one or more identical or different groups chosen from the group comprising COO(C₁-C₃-alkyl) and CONH₂;
mono-cyclic heteroaryl containing one hetero atom chosen from the group of N, O and S;
mono-cyclic heteroarylalkyl containing one or two hetero atom chosen from the group of N, O and S, wherein the alkyl radical is a C₁-C₃ alkyl radical;
mono-cyclic heteroarylacyl containing one or two hetero atoms chosen from the group of N, O and S, wherein the acyl radical is a C₁-acyl radical and the heteroaryl radical can be substituted by one or more identical or different groups chosen from the group comprising COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals may form a saturated halogen substituted or unsubstituted ring with the N atom; C(O)NH(phenyl); C(O)NH(benzyl); C(O)(C₃-C₆-cycloalkyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4:
C(O)NH—(CH)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)_{q}—COOH, wherein q is 0 or 1; C(O)—(CH₂)ᵣ—COO(C₁-C₃-alkyl), wherein r is 0 or 1; C(O)—(CH₂)ₛ—C(O)NH₂, wherein s is 0 or 1: C(O)—(CH₂)ₜ—C(O)NH(C₁-C₃-alkyl), wherein t is 0 or 1; C(O)—(CH₂)C(O)N(C₁-C₃-alkyl)₂, wherein u is 0 or 1;
C(O)—(CH₂)ᵥ—NH₂, wherein v is 0 or 1; C(O)—(CH₂)_{w}—OR′, wherein w is 0 or 1 and R′ is H or acetyl; C(O)—(CH₂)ₓ—C(O)NH—(CH₂)_{y}C(O)NH₂, wherein x is 0 or 1 and wherein y is 0 or 1;
SO₂(C₁-C₆-alkyl); SO₂—(CH₂)_{z}-heteroaryl, wherein is 0 or 1; SO₂(CH₂)ₐ-heterocyclyl, wherein a is 0 or 1, wherein the heteroatoms are O, N, and/or S and wherein the heterocyclyl residue may be substituted by one or more identical or different substituents chosen from the group comprising F, Cl, OH, CN, oxo and/or C₁-C₃-alkoxy; SO₂N(C₁-C₃-alkyl)₂ or SO₂NH(C₁-C₃-alkyl), wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);
R² and R³ form, together with the nitrogen to which they are bonded, a mono-unsaturated 6-membered N-heterocycle, that may be substituted by one or more of F, Cl, OH, CONH₂, CN, and/or O—C(O)(C₁-C₃ alkyl);
Z is chosen from the group comprising
phenyl, which can be substituted by one or more identical or different groups chosen from the group comprising F, Cl, C₁-C₃-alkyl, C₁-C₃-alkoxy, OH, CF₃, and NO₂, wherein two OH substituents may be connected by an ether bridge to form a ring or wherein two C₁-C₃-alkyl groups may be connected to form a saturated ring; and
a mono- or bicyclic aryl or heteroaryl containing one hetero atom chosen from the group of N and S, wherein the aryl or heteroaryl group can be substituted by one or more identical or different groups chosen from the group comprising F, Cl, C₁-C₃-alkyl, C₁-C₃-alkoxy, OH, CF₃, and NO₂.

5. The compound according to claim 1, wherein in general formula (1):
R¹ is chosen from the group comprising H; C₁-C₃-alkyl; COO(C₁-C₄-alkyl);
benzyl;
C₁-C₄-acyl; C(O)C₄-C₆-cycloalkyl; heterocyclylacyl containing NH or O in the ring; phenylacyl, wherein the acyl radical is a C₁-acyl radical and the phenyl radical can be substituted by one or more identical or different groups Chosen from the group comprising COO(C₁-C₃-alkyl) and CONH₂;
mono-cyclic heteroaryl containing one hetero atom chosen from the group of N, O and S;
mono-cyclic heteroarylalkyl containing one or two hetero atom chosen from the group of N, O and S, wherein the alkyl radical is a C₁-C₃ alkyl radical;
mono-cyclic heteroarylacyl containing one or two hetero atoms chosen from the group of N, O and S, wherein the acyl radical is a C₁-acyl radical and the heteroaryl radical can he substituted by one or more identical or different groups chosen from the group comprising COO(C₁-C₃-alkyl) and CONH₂;
mono-cyclic (heteroaryl)alkenylacyl containing one hetero atom chosen from the group of N, O and S, wherein the acyl radical is a C₁-acyl radical and the alkenyl radical is a C₂-C₄-alkenyl radical;
C(O)NH(C₁-C₃-alkyl); C(O)N(C₁-C₃-alkyl)₂, wherein the two alkyl radicals may for a saturated halogen substituted or unsubstituted ring with the N atom; C(O)NH(phenyl); C(O)NH(benzyl); C(O)(C₃-C₆-cycloalkyl); COO(benzyl);
(CH₂)_{g}—COOH, wherein g is 1, 2, 3 or 4; (CH₂)ₕ—COO(C₁-C₆-alkyl), wherein h is 1, 2, 3 or 4; (CH₂)ᵢ—CONH₂, wherein i is 1, 2, 3 or 4;
C(O)NH—(CH₂)ⱼ—COOH, wherein j is 0 or 1; C(O)NH—(CH₂)ₖ—COO(C₁-C₃-alkyl), wherein k is 0 or 1; C(O)NH—(CH₂)ₗ—CONH₂, wherein l is 0 or 1;
COO—(CH₂)ₘ—COOH, wherein m is 0 or 1; COO—(CH₂)ₙ—COO(C₁-C₃-alkyl), wherein n is 0 or 1; COO—(CH₂)ₚ—C(O)NH₂, wherein p is 0 or 1; C(O)—(CH₂)_{q}—COOH, wherein q is 0 or 1; C(O)—(CH₂)ᵣ—COO(C₁-C₃-alkyl), wherein r is 0 or 1; C(O)—(CH₂)ₛ—C(O)NH₂, wherein s is 0 or 1; C(O)—(CH₂)ₜ—C(O)NH(C₁-C₃-alkyl) wherein t is 0 or 1; C(O)—(CH₂)ᵤ—C(O)N(C₁-C₃-alkyl)₂, wherein u is 0 or 1;
C(O)—(CH₂)ᵥ—NH₂, wherein v is 0 or 1; C(O)—(CH₂)_{w}—OR′, wherein w is 0 or 1 and R′ is H or acetyl; C(O)—(CH₂)ₓ—C(O)NH—(CH₂)_{y}C(O)NH₂, wherein x is 0 or 1 and wherein y is 0 or 1;
SO₂(C₁-C₆-alkyl); SO₂—(CH₂)_{z}-heteroaryl, wherein z is 0 or 1; SO₂(CH₂)ₙ-heterocyclyl, wherein a is Our 1, wherein the heteroatoms are O, N, and/or S and wherein the heterocyclyl residue may be substituted by one or more identical or different substituents chosen from the group comprising F, Cl, OH, CN, oxo and/or C₁-C₃-alkoxy; SO₂N(C₁-C₃-alkyl)₂ or SO₂NH(C₁-C₃-alkyl) wherein the alkyl radical can be substituted by F, Cl, C₁-C₃-alkoxy and/or OH; SO₂NH(C₃-C₆-cycloalkyl); SO₂NH—C(O)O(C₁-C₃-alkyl);
R², R³ are identical or different and are chosen from the group comprising H, methyl, ethyl, n-propyl, and i-propyl
or
R² and R³ form, together with the nitrogen to which they are bonded, a saturated or mono-unsaturated 4- to 6-membered N-heterocycle, wherein this can be substituted by one or more identical or different groups chosen from the group comprising F, Cl, OH, CONH₂, CN, and/or O—C(O)(C₁-C₃ alkyl);
Z is either a tetrahydronaphthyl or a 2,3-dihydrobenzo-1,4-dioxinyl residue, optionally substituted by one or more of F, Cl, C₁-C₃-alkyl, C₁-C₃-alkoxy, OH, CF₃, and NO₂.

6. The compound of claim 1 for use as a medicament.

7. The compound for use as a medicament as claimed in claim 6 for the therapeutic and/or prophylactic treatment of diseases chosen from the group comprising pain-related diseases, pruritus-related diseases, and/or inflammatory diseases.

8. The compound for use as a medicament as claimed in claim 7, characterized in that the pain-related diseases are chosen from the group comprising back pain, facial pain, headaches, migraine, joint pain, muscular pain syndromes, inflammatory pain-related diseases, neuropathic pain, peripheral pain, peripheral nerve damage, visceral pain, abdominal pain, menstruation symptoms, kidney- and gallstone pain, pruritus, cancer and tumor pain, sympathetic pain, postoperative pain, postraumatic pain, hyperalgesia and/or inflammatory pain.

9. The compound for use as a medicament as claimed in claim 7, characterized in that the inflammatory diseases are chosen from the group comprising inflammatory diseases of the gastrointestinal tract, in particular inflammatory bowel diseases, such as Crohn's disease and/or colitis ulcerosa, acute or chronic inflammatory changes with inflammation of the gall bladder, inflammatory pseudopolyps, colitis cystica profunda, pneumatosis cystoides intestinales, pancreatitis, appendicitis, cardiovascular inflammation due to arthereosclerosis, ischemia, restenosis and/or vasculitis, sepsis, septicemia, allergies, asthma, Sjogren's syndrome, pulmonary inflammation, chronic airway inflammation, chronic obstructive pulmonary disease (COPD), tumor proliferation, tumor metastasis, transplant rejection, inflammatory diseases of the joints, such as rheumatoid arthritis, vulvovaginitis, and/or inflammatory diseases of the brain, skin, hair follicle, urogenital tract and of the eyes, sinusitis, tenosynovitis, bursitis, tendonitis, lateral epicondylitis, adhesive capsulitis, osteomyelitis, osteoarthritic inflammation, ocular inflammation, otitic inflammation and/or autoimmune inflammation.

10. The compound for use as a medicament as claimed in claim 7, characterized in that the pruritus-related diseases are chosen from the group comprising pruritus, psoriasis, psoriatic arthritis, contact dermatitis, atopic eczema, scleroderma and other fibrotic diseases, systemic lupus erythematous, urticaria, lichen planus, lymphoma and/or allergic diseases or characterized by mast cell involvements.

11. The compound for use as a medicament as claimed in claim 6 for therapeutic and/or prophylactic treatment of hyponatremia, edema, ileus, tussis, glaucoma, multiple sclerosis, Morbus Parkinson and Morbus Alzheimer.

12. A medicament comprising at least one compound as claimed in claim 1 or a solvate or hydrate thereof or a pharmaceutically acceptable salt thereof.

13. The medicament as claimed in claim 12, further comprising at least one opioid receptor antagonist, preferably chosen from the group comprising naloxone, naltrexone cyprodime, naltrindole, norbinaltorphimine nalmefene, nalorphine, nalbuphine, naloxonazine, methylnaltrexone and/or ketylcyclazocine, and/or a steroidal anti-inflammatory drug, preferably chosen from the group of hydrocortisone, hydrocortisone acetate, prednisolone, methylprednisolone, prednisone, betamethasone, hydrocortisone-17-valerate, betamethasone valerate, betamethasone dipropionate, prednicarbate, clobetasone-17-butyrate flunisolide, fluticasone propionate, triamcinolone acetonide, beclomethasone dipropionate, budesonide and/or hydrocortisone-17-butyrate and/or a nonsteroidal anti-inflammatory drug (NSAID), preferably chosen from the group of aspirin, ibuprofen, diclofenac and/or naproxen, and/or an opioid receptor agonist, preferably chosen from the group comprising tramadol, pethidin, codein, piritramid, morphin, levomethadon, fentanyl, alfentanil, remifentanil and/or sufentanil, and/or an antibiotic.

14. A process for the preparation of a compound according to the general formula (1) as claimed in claim 1, characterized in that the process comprises the following steps:
a) reacting 5,6,7,8-tetrahydroquinoxalin-5-ol with a protection agent X-PG in the presence of a base to introduce a protecting group PG at the alcohol function, wherein X is a suitable leaving group;
b) catalytically hydrogenating the PG protected 5,6,7,8-tetrahydroquinoxalin-5-ol obtained in step a) under stereoselective reduction of the pyrazine ring to obtain PG protected cis-cis 5-hydroxy-decahydroquinoxaline;
c) reacting the PG protected cis-cis 5-hydroxy-decahydroquinoxaline obtained in step b) with a reagent X—R¹ to regioselectively introduce the substituent R¹ at the 1-N atom of the cis-cis 5-hydroxy-decahydroquinoxaline, wherein X is a suitable leaving group;
d) deprotecting the PG protected hydroxy group in the product obtained in step c) to provide for the corresponding α,β-aminoalcohol;
e) reacting the α,β-aminoalcohol obtained in step d) with sulfuryl chloride in the presence of a base to provide for the corresponding 1,2,3-oxathiazolidine 2,2-dioxide;
f) reacting the 1,2,3-oxathiazolidine 2,2-dioxide obtained in step e) with an amine HNR²R³, followed by treatment with an acid to introduce the residue —NR²R³ under inversion of the stereogenic center to provide for cis,trans 5-amino-octahydroquinoxaline; and
g) reacting the cis,trans 5-amino-octahydroquinoxaline obtained in step f) with an activated carboxylic acid derivative ZCH₂COY, wherein Y is a suitable leaving group, preferably with an acid chloride Z—CH₂COCl, under acylation in 4-position to provide for the compound of formula (1) together with its enantiomeric form.

15. The process according to claim 14, farther comprising the following reaction steps (a1) and (a2) carried out before step a):
(a1) oxidizing 5,6,7,8-tetrahydroquinoxalin-5-ol to the corresponding ketone with an oxidizing agent;
(a2) subjecting the ketone obtained in step (a1) to an asymmetric hydrogen transfer reaction using a hydrogenation agent and a chiral catalyst to provide for enantiomerically pure (R)-5,6,7,8-tetrahydroquinoxalin-5-ol,
subjecting the (R)-5,6,7,8-tetrahydroquinoxalin-5-ol obtained in step (a2) to the reaction steps a) to i),
to provide for compounds of formula (1) in enantiomerically pure form.

16. The process according to claim 14, wherein the process further comprises the step of:
h) separating the compound of formula (1) from its enantiomeric form.

17. The process according to claim 16, wherein the process further comprises the step of:
i) converting the compound of formula (1) obtained in step g) or step h) to pharmaceutically acceptable salts by reaction with the corresponding acid.

18. The process according to claim 15, wherein the process further comprises the step of:
h) separating the compound of formula (1) from its enantiomeric form.

19. The process according to claim 18, wherein the process further comprises the step of:
i) converting the compound of formula (1) obtained in step g) or step h) to pharmaceutically acceptable salts by reaction with the corresponding acid.
